(19) United States
(12) Patent Application Publication
(10) Pub. No.: US 2026/0285830 A1
(43) Pub. Date: Sep. 24, 2026

(54) CRYSTALLINE METHIONINE ADENOSYLTRANSFERASE 2a (MAT2A) INHIBITOR AND USES THEREOF

(71) Applicant: Insilico Medicine IP Limited, Hong Kong (CN)

(72) Inventors: Xing LIANG, Shanghai (CN); Hui WANG, Shanghai (CN); Yushu YIN, Shanghai (CN); Congcong ZHU, Shanghai (CN); Chiachun CHEN, Shanghai (CN); Xiao DING, Shanghai (CN)

(21) Appl. No.: 19/475,381

(22) PCT Filed: Apr. 18, 2024
(86) PCT No.: PCT/CN2024/088575

§ 371 Date: Oct. 14, 2025

(30) Foreign Application Priority Data
Apr. 19, 2023 (WO) PCT/CN2023/089331

(51) Int. Cl. C07D 401/04; A61K 31/517; A61P 35/00; C07C 57/145; C07C 59/265

(52) U.S. Cl. CPC C07D 401/04; A61K 31/517; A61P 35/00; C07C 57/145; C07C 59/265

(57) ABSTRACT

Described herein are crystalline forms of a small molecule methionine adenosyltransferase 2a (MAT2A) inhibitor, as well as pharmaceutical compositions thereof, and methods of use thereof in the treatment of diseases or conditions that would benefit from treatment with methionine adenosyltransferase 2a (MAT2A) inhibitor.

### CROSS-REFERENCE

This patent application claims the benefit of International Application No. PCT/CN2023/089331, filed Apr. 19, 2023; which is incorporated herein by reference in its entirety.

### BACKGROUND

Methionine adenosyltransferase 2a (MAT2A) plays an important role in metabolism and epigenetics. Despite its broad cellular role, inhibition of MAT2A has been shown to result in a selective anti-proliferative effect in cancers with deletion of a separate metabolic gene, methylthioadenosine phosphorylase (“MTAP”). MTAP deficiency occurs frequently in both solid tumors and hematologic malignancies. As such, compounds that inhibit MAT2A are potential agents for treating MTAP-deficient cancers.

### SUMMARY

Disclosed herein is a solid state form of 5-methoxy-1-(2-methylpyridin-3-yl)-4-(prop-2-yn-1-ylamino)-7-(trifluoromethyl) quinazolin-2 (1H)-one:

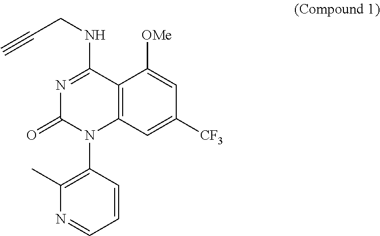 [SMILES: C#CCNc1nc(=O)n(-c2cccnc2C)c2cc(C(F)(F)F)cc(OC)c12]

or a pharmaceutically acceptable salt thereof.

In some embodiments, the solid state form is a crystalline form.

In some embodiments, the solid state form is crystalline Compound 1 freebase Type A.

In some embodiments, the solid state form is crystalline Compound 1 freebase Type B, crystalline Compound 1 freebase Type C, crystalline Compound 1 HCl salt Type A, crystalline Compound 1 HCl salt Type B, crystalline Compound 1 HCl salt Type C, crystalline Compound 1 sulfate salt Type A, crystalline Compound 1 phosphate salt Type A, crystalline Compound 1 L-malate salt Type A, crystalline Compound 1 succinate salt Type A, crystalline Compound 1 tosylate salt Type A, crystalline Compound 1 tosylate salt Type B, crystalline Compound 1 tosylate salt Type C, crystalline Compound 1 mesylate salt Type A, crystalline Compound 1 besylate salt Type A, crystalline Compound 1 oxalate salt Type A, crystalline Compound 1 oxalate salt Type B, crystalline Compound 1 esylate salt Type A, or crystalline Compound 1 esylate salt Type B.

In some embodiments, the solid state form is Compound 1 maleate salt.

In some embodiments, the solid state form is crystalline Compound 1 maleate salt Type A.

In some embodiments, the solid state form is crystalline Compound 1 citrate salt Type A.

Also disclosed herein is a pharmaceutical composition comprising a crystalline form disclosed herein and at least one pharmaceutically acceptable excipient.

Also disclosed herein is a method of treating cancer in a mammal in need thereof, comprising administering to the mammal a crystalline form disclosed herein or pharmaceutical composition disclosed herein.

In some embodiments, the cancer is a MTAP-deficient cancer.

In some embodiments, the cancer is selected from liver cancer, colon cancer, pancreatic cancer, prostate cancer, lung cancer, breast cancer, gastrointestinal stromal tumor, biliary tract cancer, acute lymphoblastic leukemia (ALL) B-lineage, lymphoma, or T cell leukemia.

### BRIEF DESCRIPTION OF THE DRAWINGS

The features of the invention are set forth with particularity in the appended claims. A better understanding of the features of the present invention will be obtained by reference to the following detailed description that sets forth illustrative embodiments, in which the principles of the invention are utilized, and the accompanying drawings of which:

FIG. 1 shows the X-Ray Powder Diffraction (XRPD) pattern of Compound 1 freebase Type A.

FIG. 2 shows the Thermogravimetric Analysis (TGA) thermogram and Differential Scanning calorimetry (DSC) thermogram of Compound 1 freebase Type A.

FIG. 3 shows the X-Ray Powder Diffraction (XRPD) pattern of Compound 1 freebase Type B.

FIG. 4 shows the Thermogravimetric Analysis (TGA) thermogram and Differential Scanning calorimetry (DSC) thermogram of Compound 1 freebase Type B.

FIG. 5 shows the X-Ray Powder Diffraction (XRPD) pattern of Compound 1 freebase Type C.

FIG. 6 shows the Thermogravimetric Analysis (TGA) thermogram and Differential Scanning calorimetry (DSC) thermogram of Compound 1 freebase Type C.

FIG. 7 shows the X-Ray Powder Diffraction (XRPD) pattern of Compound 1 HCl salt Type A.

FIG. 8 shows the Thermogravimetric Analysis (TGA) thermogram and Differential Scanning calorimetry (DSC) thermogram of HCl salt Type A.

FIG. 9 shows the X-Ray Powder Diffraction (XRPD) pattern of Compound 1 HCl salt Type B.

FIG. 10 shows the Thermogravimetric Analysis (TGA) thermogram and Differential Scanning calorimetry (DSC) thermogram of Compound 1 HCl salt Type B.

FIG. 11 shows the X-Ray Powder Diffraction (XRPD) pattern of Compound 1 HCl salt Type C.

FIG. 12 shows the Thermogravimetric Analysis (TGA) thermogram and Differential Scanning calorimetry (DSC) thermogram of Compound 1 HCl salt Type C.

FIG. 13 shows the X-Ray Powder Diffraction (XRPD) pattern of Compound 1 sulfate salt Type A.

FIG. 14 shows the Thermogravimetric Analysis (TGA) thermogram and Differential Scanning calorimetry (DSC) thermogram of Compound 1 sulfate salt Type A.

FIG. 15 shows the X-Ray Powder Diffraction (XRPD) pattern of Compound 1 maleate salt Type A.

FIG. 16 shows the Thermogravimetric Analysis (TGA) thermogram and Differential Scanning calorimetry (DSC) thermogram of Compound 1 maleate salt Type A.

FIG. 17 shows the ¹H NMR spectrum of Compound 1 maleate salt Type A.

FIG. 18 shows the X-Ray Powder Diffraction (XRPD) pattern of Compound 1 phosphate salt Type A.

FIG. 19 shows the Thermogravimetric Analysis (TGA) thermogram and Differential Scanning calorimetry (DSC) thermogram of Compound 1 phosphate salt Type A.

FIG. 20 shows the X-Ray Powder Diffraction (XRPD) pattern of Compound 1 citrate salt Type A.

FIG. 21 shows the Thermogravimetric Analysis (TGA) thermogram and Differential Scanning calorimetry (DSC) thermogram of Compound 1 citrate salt Type A.

FIG. 22 shows the ¹H NMR spectrum of Compound 1 citrate salt Type A.

FIG. 23 shows the X-Ray Powder Diffraction (XRPD) pattern of Compound 1 L-malate salt Type A.

FIG. 24 shows the Thermogravimetric Analysis (TGA) thermogram and Differential Scanning calorimetry (DSC) thermogram of Compound 1 L-malate salt Type A.

FIG. 25 ¹H NMR spectrum of Compound 1 L-malate salt Type A.

FIG. 26 shows the X-Ray Powder Diffraction (XRPD) pattern of Compound 1 succinate salt Type A.

FIG. 27 shows the Thermogravimetric Analysis (TGA) thermogram and Differential Scanning calorimetry (DSC) thermogram of Compound 1 succinate salt Type A.

FIG. 28 shows the ¹H NMR spectrum of Compound 1 succinate salt Type A.

FIG. 29 shows the X-Ray Powder Diffraction (XRPD) pattern of Compound 1 Tosylate salt Type A.

FIG. 30 shows the Thermogravimetric Analysis (TGA) thermogram and Differential Scanning calorimetry (DSC) thermogram of Compound 1 tosylate salt Type A.

FIG. 31 shows the ¹H NMR spectrum of Compound 1 tosylate salt Type A.

FIG. 32 shows the X-Ray Powder Diffraction (XRPD) pattern of Compound 1 tosylate salt Type B.

FIG. 33 shows the Thermogravimetric Analysis (TGA) thermogram and Differential Scanning calorimetry (DSC) thermogram of Compound 1 tosylate salt Type B.

FIG. 34 shows the ¹H NMR spectrum of Compound 1 tosylate salt Type B.

FIG. 35 shows the X-Ray Powder Diffraction (XRPD) pattern of Compound 1 tosylate salt Type C.

FIG. 36 shows the Thermogravimetric Analysis (TGA) thermogram and Differential Scanning calorimetry (DSC) thermogram of Compound 1 tosylate salt Type C.

FIG. 37 shows the ¹H NMR spectrum of Compound 1 tosylate salt Type C.

FIG. 38 shows the X-Ray Powder Diffraction (XRPD) pattern of Compound 1 mesylate salt Type A.

FIG. 39 shows the Thermogravimetric Analysis (TGA) thermogram and Differential Scanning calorimetry (DSC) thermogram of Compound 1 mesylate salt Type A.

FIG. 40 shows the ¹H NMR spectrum of Compound 1 mesylate salt Type A.

FIG. 41 shows the X-Ray Powder Diffraction (XRPD) pattern of Compound 1 besylate salt Type A.

FIG. 42 shows the Thermogravimetric Analysis (TGA) thermogram and Differential Scanning calorimetry (DSC) thermogram of Compound 1 besylate salt Type A.

FIG. 43 shows the ¹H NMR spectrum of Compound 1 besylate salt Type A.

FIG. 44 shows the X-Ray Powder Diffraction (XRPD) pattern of Compound 1 oxalate salt Type A.

FIG. 45 shows the Thermogravimetric Analysis (TGA) thermogram and Differential Scanning calorimetry (DSC) thermogram of Compound 1 oxalate salt Type A.

FIG. 46 shows the X-Ray Powder Diffraction (XRPD) pattern of Compound 1 oxalate salt Type B.

FIG. 47 shows the Thermogravimetric Analysis (TGA) thermogram and Differential Scanning calorimetry (DSC) thermogram of Compound 1 oxalate salt Type B.

FIG. 48 shows the X-Ray Powder Diffraction (XRPD) pattern of Compound 1 esylate salt Type A.

FIG. 49 shows the Thermogravimetric Analysis (TGA) thermogram and Differential Scanning calorimetry (DSC) thermogram of Compound 1 esylate salt Type A.

FIG. 50 shows the ¹H NMR spectrum of Compound 1 esylate salt Type A.

FIG. 51 shows the X-Ray Powder Diffraction (XRPD) pattern of Compound 1 esylate salt Type B.

FIG. 52 shows the Thermogravimetric Analysis (TGA) thermogram and Differential Scanning calorimetry (DSC) thermogram of Compound 1 esylate salt Type B.

FIG. 53 shows the ¹H NMR spectrum of esylate salt Type B.

FIG. 54 shows the Dynamic Vapor Sorption (DVS) plot of Compound 1 freebase Type A.

FIG. 55 shows the Dynamic Vapor Sorption (DVS) plot of Compound 1 freebase Type B.

FIG. 56 shows the Dynamic Vapor Sorption (DVS) plot of Compound 1 freebase Type C.

FIG. 57 shows the Dynamic Vapor Sorption (DVS) plot of Compound 1 maleate salt Type A.

FIG. 58 shows the Dynamic Vapor Sorption (DVS) plot of Compound 1 citrate salt Type A.

FIG. 59A shows the solubility of Compound Freebase Type A, Compound 1 citrate salt Type A, and Compound 1 Maleate salt Type A in H₂O at 37 [symbol] .

FIG. 59B shows the solubility of Compound Freebase Type A, Compound 1 citrate salt Type A, and Compound 1 Maleate salt Type A in SGF at 37 [symbol] .

FIG. 59C shows the solubility of Compound Freebase Type A, Compound 1 citrate salt Type A, and Compound 1 Maleate salt Type A in FaSSIF at 37 [symbol] .

FIG. 59D shows the solubility of Compound Freebase Type A, Compound 1 citrate salt Type A, and Compound 1 Maleate salt Type A in FeSSIF at 37 [symbol] .

### DETAILED DESCRIPTION

While small molecule inhibitors are often initially evaluated for their activity when dissolved in solution, solid state characteristics such as polymorphism are also important. Polymorphic forms of a drug substance can have different physical properties, including melting point, apparent solubility, dissolution rate, optical and mechanical properties, vapor pressure, and density. These properties can have a direct effect on the ability to process or manufacture a drug substance and the drug product. Moreover, differences in these properties can and often lead to different pharmacokinetics profiles for different polymorphic forms of a drug. Therefore, polymorphism is often an important factor under regulatory review of the ‘sameness’ of drug products from various manufacturers.

### Compound 1

Compound 1 is 5-methoxy-1-(2-methylpyridin-3-yl)-4-(prop-2-yn-1-ylamino)-7-(trifluoromethyl) quinazolin-2 (1H)-one:

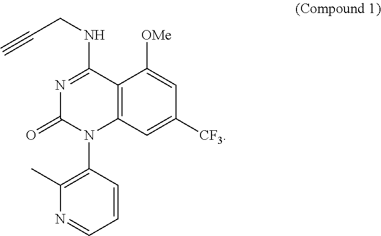 [SMILES: C#CCNc1nc(=O)n(-c2cccnc2C)c2cc(C(F)(F)F)cc(OC)c12]

In some embodiments, Compound 1 is in the form of a freebase. In some embodiments, Compound 1 is in the form of a pharmaceutically acceptable salt. In some embodiments, Compound 1 is in the form of an HCl salt. In some embodiments, Compound 1 is in the form of a sulfate salt. In some embodiments, Compound 1 is in the form of a maleate salt. In some embodiments, Compound 1 is in the form of a phosphate salt. In some embodiments, Compound 1 is in the form of a citrate salt. In some embodiments, Compound 1 is in the form of a L-malate salt. In some embodiments, Compound 1 is in the form of a succinate salt. In some embodiments, Compound 1 is in the form of a tosylate salt.

In some embodiments, Compound 1 is in the form of a mesylate salt. In some embodiments, Compound 1 is in the form of a besylate salt. In some embodiments, Compound 1 is in the form of an oxalate salt. In some embodiments, Compound 1 is in the form of an esylate salt.

### Solid State Form of Compound 1

In one aspect, provided herein is a solid state form of 5-methoxy-1-(2-methylpyridin-3-yl)-4-(prop-2-yn-1-ylamino)-7-(trifluoromethyl) quinazolin-2 (1H)-one:

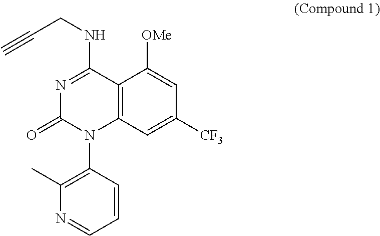 [SMILES: C#CCNc1nc(=O)n(-c2cccnc2C)c2cc(C(F)(F)F)cc(OC)c12]

or a pharmaceutically acceptable salt thereof.

In some embodiments, the solid state form is a crystalline form.

In some embodiments, the solid state form is crystalline Compound 1 freebase. In some embodiments, the solid state form is crystalline Compound 1 freebase Type A. In some embodiments, the solid state form is crystalline Compound 1 freebase Type B. In some embodiments, the solid state form is crystalline Compound 1 freebase Type C.

In some embodiments, the solid state form is Compound 1 HCl salt. In some embodiments, the solid state form is crystalline Compound 1 HCl salt. In some embodiments, the solid state form is crystalline Compound 1 HCl salt Type A. In some embodiments, the solid state form is crystalline Compound 1 HCl salt Type B. In some embodiments, the solid state form is crystalline Compound 1 HCl salt Type C.

In some embodiments, the solid state form is Compound 1 sulfate salt. In some embodiments, the solid state form is crystalline Compound 1 sulfate salt. In some embodiments, the solid state form is crystalline Compound 1 sulfate salt Type A.

In some embodiments, the solid state form is Compound 1 maleate salt. In some embodiments, the solid state form is crystalline Compound 1 maleate salt. In some embodiments, the solid state form is crystalline Compound 1 maleate salt Type A.

In some embodiments, the solid state form is Compound 1 phosphate salt. In some embodiments, the solid state form is crystalline Compound 1 phosphate salt. In some embodiments, the solid state form is crystalline Compound 1 phosphate salt Type A.

In some embodiments, the solid state form is Compound 1 citrate salt. In some embodiments, the solid state form is crystalline Compound 1 citrate salt. In some embodiments, the solid state form is crystalline Compound 1 citrate salt Type A.

In some embodiments, the solid state form is Compound 1 L-malate salt. In some embodiments, the solid state form is crystalline Compound 1 L-malate salt. In some embodiments, the solid state form is crystalline Compound 1 L-malate salt Type A.

In some embodiments, the solid state form is Compound 1 succinate salt. In some embodiments, the solid state form is crystalline Compound 1 succinate salt. In some embodiments, the solid state form is crystalline Compound 1 succinate salt Type A.

In some embodiments, the solid state form is Compound 1 tosylate salt. In some embodiments, the solid state form is crystalline Compound 1 tosylate salt. In some embodiments, the solid state form is crystalline Compound 1 tosylate salt Type A. In some embodiments, the solid state form is crystalline Compound 1 tosylate salt Type B. In some embodiments, the solid state form is crystalline Compound 1 tosylate salt Type C.

In some embodiments, the solid state form is Compound 1 mesylate salt. In some embodiments, the solid state form is crystalline Compound 1 mesylate salt. In some embodiments, the solid state form is crystalline Compound 1 mesylate salt Type A.

In some embodiments, the solid state form is Compound 1 besylate salt. In some embodiments, the solid state form is crystalline Compound 1 besylate salt. In some embodiments, the solid state form is crystalline Compound 1 besylate salt Type A.

In some embodiments, the solid state form is Compound 1 oxalate salt. In some embodiments, the solid state form is crystalline Compound 1 oxalate salt. In some embodiments, the solid state form is crystalline Compound 1 oxalate salt Type A. In some embodiments, the solid state form is crystalline Compound 1 oxalate salt Type B.

In some embodiments, the solid state form is Compound 1 esylate salt. In some embodiments, the solid state form is crystalline Compound 1 esylate salt. In some embodiments, the solid state form is crystalline Compound 1 esylate salt Type A. In some embodiments, the solid state form is crystalline Compound 1 esylate salt Type B.

In some embodiments, the solid state form is crystalline Compound 1 freebase Type B, crystalline Compound 1 freebase Type C, crystalline Compound 1 HCl salt Type A, crystalline Compound 1 HCl salt Type B, crystalline Compound 1 HCl salt Type C, crystalline Compound 1 sulfate salt Type A, crystalline Compound 1 phosphate salt Type A, crystalline Compound 1 L-malate salt Type A, crystalline Compound 1 succinate salt Type A, crystalline Compound 1 tosylate salt Type A, crystalline Compound 1 tosylate salt Type B, crystalline Compound 1 tosylate salt Type C, crystalline Compound 1 mesylate salt Type A, crystalline Compound 1 besylate salt Type A, crystalline Compound 1 oxalate salt Type A, crystalline Compound 1 oxalate salt Type B, crystalline Compound 1 esylate salt Type A, or crystalline Compound 1 esylate salt Type B.

### Compound 1 Freebase Type A

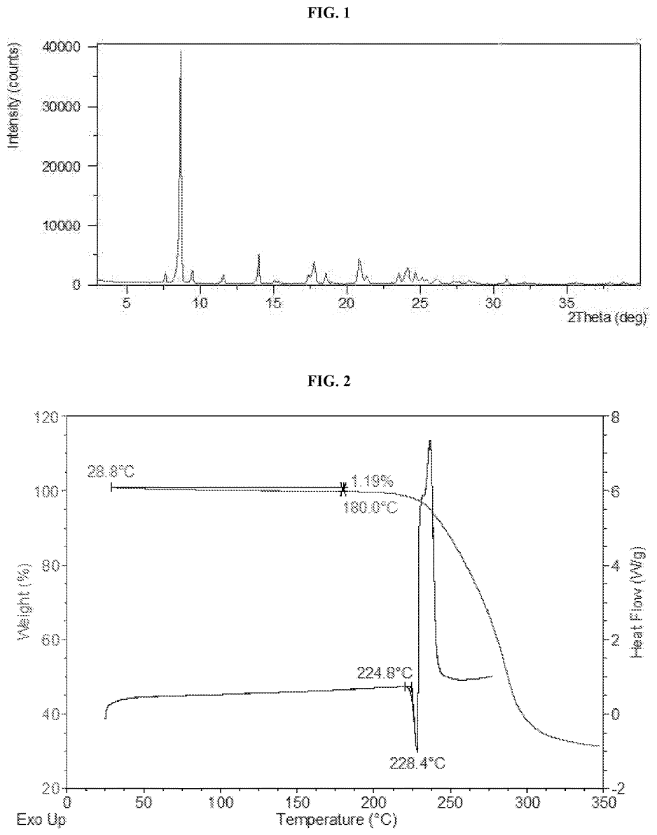

Disclosed herein is Compound 1 freebase Type A. In some embodiments, the crystalline form is Compound 1 freebase Type A characterized as having at least one of the following properties:
(a) an X-Ray powder diffraction (XRPD) pattern substantially the same as shown in FIG. 1 as measured using Cu Kα.radiation;
(b) an X-Ray powder diffraction (XRPD) pattern with peaks at 8.7±0. [symbol] 2θ and 14.0±0.2° 2θ as measured using Cu Kα.radiation;
(c) a Differential Scanning calorimetry (DSC) thermogram substantially the same as shown in FIG. 2;
(d) a Differential Scanning calorimetry (DSC) thermogram with an endothermic peak having an peak temperature at about 228.4 [symbol] ;
(e) a Thermogravimetric Thermal Analysis (TGA) thermogram substantially the same as shown in FIG. 2;
(f) a Thermogravimetric Thermal Analysis (TGA) thermogram exhibiting a mass loss of about 1.2% from the onset of heating up to approximately 180 [symbol] ; or
(g) combinations thereof.

In some embodiments, the crystalline form is Compound 1 freebase Type A characterized as having at least one of the following properties:
(a) an X-Ray powder diffraction (XRPD) pattern substantially the same as shown in FIG. 1 as measured using Cu Kα.radiation;
(b) an X-Ray powder diffraction (XRPD) pattern with peaks at 8.7±0.2° 2θ and 14.0±0.2° 20 as measured using Cu Kα.radiation;
(c) a Differential Scanning calorimetry (DSC) thermogram substantially the same as shown in FIG. 2;
(d) a Thermogravimetric Thermal Analysis (TGA) thermogram substantially the same as shown in FIG. 2; or
(e) combinations thereof.

In some embodiments, the crystalline form is Compound 1 freebase Type A characterized as having at least one of the following properties:
(a) an X-Ray powder diffraction (XRPD) pattern with peaks at 8.7±0.2° 2θ and 14.0═0.2° 2θ as measured using Cu Kα.radiation;
(b) a Differential Scanning calorimetry (DSC) thermogram with an endothermic peak having an peak temperature at about 228.4 [symbol] ;
(c) a Thermogravimetric Thermal Analysis (TGA) thermogram exhibiting a mass loss of about 1.2% from the onset of heating up to approximately 180 [symbol] ; or
(d) combinations thereof.

In some embodiments of Compound 1 freebase Type A, the crystalline form has an X-ray powder diffraction (XRPD) pattern substantially the same as shown in FIG. 1 as measured using Cu Kα.radiation.

In some embodiments of Compound 1 freebase Type A, the crystalline form has an X-ray powder diffraction (XRPD) pattern with characteristic peaks found in Table 1 as measured using Cu Kα.radiation.

In some embodiments of Compound 1 freebase Type A, the crystalline form has an X-ray powder diffraction (XRPD) pattern with peaks at 8.7±0.2° 2θ and 14.0±0.2° 2θ as measured using Cu Kα.radiation.

In some embodiments of Compound 1 freebase Type A, the X-ray powder diffraction (XRPD) pattern further comprises peaks at 7.7±0.2° 2θ, 9.5±0.2° 2θ, and 11.6±0.2° 2θ as measured using Cu Kα.radiation.

In some embodiments of Compound 1 freebase Type A, the X-ray powder diffraction (XRPD) pattern further comprises peaks at 17.8±0.2° 2θ, 20.8±0.2° 2θ, and 24.2±0.2° 2θ as measured using Cu Kα.radiation.

In some embodiments of Compound 1 freebase Type A, the crystalline form has an X-ray powder diffraction (XRPD) pattern with peaks at 7.7±0.2° 2θ, 8.7±0.2° 2θ, 9.5±0.2° 2θ, 14.0=0. [symbol] 2θ, 17.8±0.2° 2θ, 20.8±0.2° 2θ, and 24.2±0.2° 2θ as measured using Cu Kα.radiation.

In some embodiments of Compound 1 freebase Type A, the crystalline form has an X-ray powder diffraction (XRPD) pattern with at least two peaks selected from 7.7±0.2° 2θ, 8.7±0.2° 2θ, 9.5=±0.2° 2θ, 14.0±0.2° 2θ, 17.8±0.2° 2θ, 20.8±0.2° 2θ, and 24.2±0.2° 2θ as measured using Cu Kα.radiation.

In some embodiments of Compound 1 freebase Type A, the crystalline form has an X-ray powder diffraction (XRPD) pattern with at least three peaks selected from 7.7±0.2° 2θ, 8.7±0.2° 2θ, 9.5±0.2° 2θ, 14.0±0.2° 2θ, 17.8±0.2° 2θ, 20.8±0.2° 2θ, and 24.2±0.2° 2θ as measured using Cu Kα.radiation.

In some embodiments of Compound 1 freebase Type A, the crystalline form has an X-ray powder diffraction (XRPD) pattern with at least four peaks selected from 7.7±0.2° 2θ, 8.7±0.2° 2θ, 9.5±0.2° 2θ, 14.0±0.2° 2θ, 17.8±0.2° 2θ, 20.8±0.2° 2θ, and 24.2±0.2° 2θ as measured using Cu Kα.radiation.

In some embodiments of Compound 1 freebase Type A, the crystalline form has an X-ray powder diffraction (XRPD) pattern with at least five peaks selected from 7.7±0.2° 2θ, 8.7±0.2° 2θ, 9.5±0.2° 2θ, 14.0±0.2° 2θ, 17.8±0.2° 2θ, 20.8±0.2° 2θ, and 24.2±0.2° 2θ as measured using Cu Kα.radiation.

In some embodiments of Compound 1 freebase Type A, the crystalline form has an X-ray powder diffraction (XRPD) pattern with at least six peaks selected from 7.7±0.2° 2θ, 8.7±0.2° 2θ, 9.5±0.2° 2θ, 14.0±0.2° 2θ, 17.8±0.2° 2θ, 20.8±0.2° 200, and 24.2±0.2° 2θ as measured using Cu Kα.radiation.

In some embodiments of Compound 1 freebase Type A, the Differential Scanning calorimetry (DSC) thermogram is substantially the same as shown in FIG. 2.

In some embodiments of Compound 1 freebase Type A, the Differential Scanning calorimetry (DSC) thermogram has an endothermic peak having a peak temperature at about 228.4 [symbol] .

In some embodiments of Compound 1 freebase Type A, the Thermogravimetric Thermal Analysis (TGA) thermogram is substantially the same as shown in FIG. 2.

In some embodiments of Compound 1 freebase Type A, the Thermogravimetric Thermal Analysis (TGA) thermogram exhibits a mass loss of about 1.2% from the onset of heating up to approximately 180 [symbol] .

In some embodiments of Compound 1 freebase Type A, the crystalline form is anhydrous.

TABLE 1
X-Ray Powder Diffraction peaks of freebase Type A

| Pos. [°2θ] | Rel. Int. [%] |
|---|---|
| 7.67 | 4.50 |
| 8.69 | 100.00 |
| 9.49 | 5.72 |
| 11.58 | 3.86 |
| 14.02 | 12.90 |
| 15.10 | 1.41 |
| 15.33 | 1.41 |
| 16.52 | 0.39 |
| 17.38 | 3.96 |
| 17.79 | 9.92 |
| 18.59 | 4.46 |
| 20.84 | 11.03 |
| 21.36 | 3.48 |
| 22.33 | 0.36 |
| 23.56 | 4.75 |
| 24.17 | 7.03 |
| 24.67 | 5.40 |
| 25.14 | 2.86 |
| 25.45 | 1.87 |
| 26.10 | 2.21 |
| 27.33 | 1.22 |
| 27.69 | 1.22 |
| 28.36 | 1.65 |
| 30.89 | 2.16 |
| 32.13 | 0.72 |
| 32.65 | 0.54 |
| 35.66 | 0.61 |
| 36.02 | 0.42 |
| 37.95 | 0.79 |
| 38.88 | 0.63 |

### Compound 1 Freebase Type B

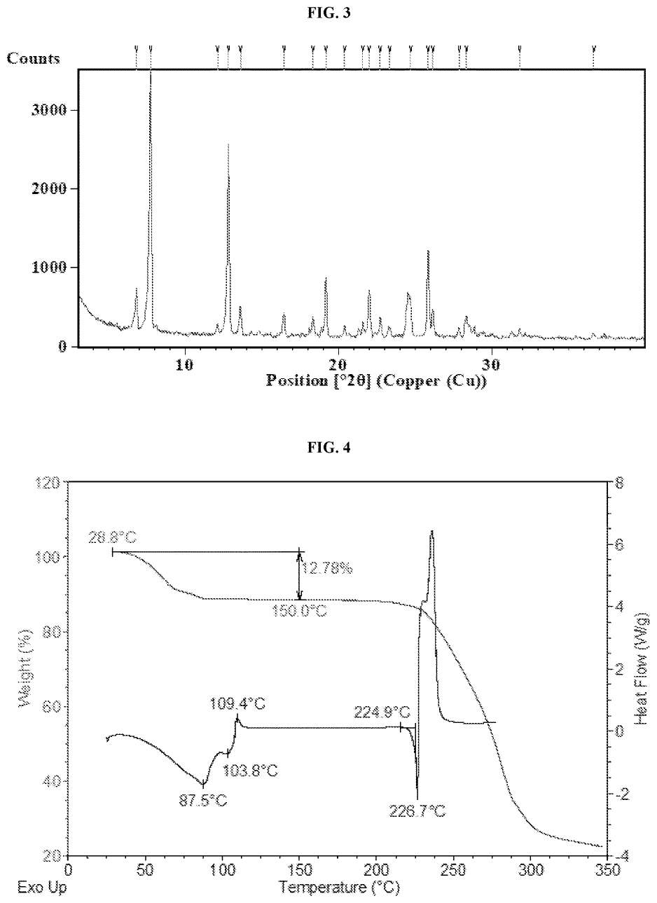

Disclosed herein is Compound 1 freebase Type B. In some embodiments, the crystalline form is Compound 1 freebase Type B characterized as having at least one of the following properties:
(a) an X-Ray powder diffraction (XRPD) pattern substantially the same as shown in FIG. 3 as measured using Cu Kα.radiation;
(b) an X-Ray powder diffraction (XRPD) pattern with peaks at 6.8±0.2° 2θ, 7.8±0.2° 2θ, and 12.8±0.2° 2θ as measured using Cu Kα.radiation;
(c) a Differential Scanning calorimetry (DSC) thermogram substantially the same as shown in FIG. 4;
(d) a Differential Scanning calorimetry (DSC) thermogram with an exothermic peak having a peak temperature at about 109.4 [symbol] ;
(e) a Differential Scanning calorimetry (DSC) thermogram with an endothermic peak having a peak temperature at about 87.5 [symbol] ;
(f) a Differential Scanning calorimetry (DSC) thermogram with an endothermic peak having a peak temperature at about 103.8 [symbol] ;
(g) a Differential Scanning calorimetry (DSC) thermogram with an endothermic peak having a peak temperature at about 226.7 [symbol] ;
(h) a Thermogravimetric Thermal Analysis (TGA) thermogram substantially the same as shown in FIG. 4;
(i) a Thermogravimetric Thermal Analysis (TGA) thermogram exhibiting a mass loss of about 12.78% from the onset of heating up to approximately 150 [symbol] ; or
(j) combinations thereof.

In some embodiments, the crystalline form is Compound 1 freebase Type B characterized as having at least one of the following properties:
(a) an X-Ray powder diffraction (XRPD) pattern substantially the same as shown in FIG. 3 as measured using Cu Kα.radiation;
(b) an X-Ray powder diffraction (XRPD) pattern with peaks at 6.8±0.2° 2θ, 7.8±0.2° 2θ, and 12.8±0.2° 2θ as measured using Cu Kα.radiation;
(c) a Differential Scanning calorimetry (DSC) thermogram substantially the same as shown in FIG. 4;
(d) a Thermogravimetric Thermal Analysis (TGA) thermogram substantially the same as shown in FIG. 4; or
(e) combinations thereof.

In some embodiments, the crystalline form is Compound 1 freebase Type B characterized as having at least one of the following properties:
(a) an X-Ray powder diffraction (XRPD) pattern with peaks at 6.8±0.2° 2θ, 7.8±0.2° 2θ, and 12.8±0.2° 2θ as measured using Cu Kα.radiation;
(b) a Differential Scanning calorimetry (DSC) thermogram with an exothermic peak having a peak temperature at about 109.4 [symbol] ;
(c) a Differential Scanning calorimetry (DSC) thermogram with an endothermic peak having a peak temperature at about 87.5 [symbol] ;
(d) a Differential Scanning calorimetry (DSC) thermogram with an endothermic peak having a peak temperature at about 103.8 [symbol] ;
(e) a Differential Scanning calorimetry (DSC) thermogram with an endothermic peak having a peak temperature at about 226.7 [symbol] ;
(f) a Thermogravimetric Thermal Analysis (TGA) thermogram exhibiting a mass loss of about 12.78% from the onset of heating up to approximately 150 [symbol] ; or
(g) combinations thereof.

In some embodiments of Compound 1 freebase Type B, the crystalline form has an X-ray powder diffraction (XRPD) pattern substantially the same as shown in FIG. 3 as measured using Cu Kα.radiation.

In some embodiments of Compound 1 freebase Type B, the crystalline form has an X-ray powder diffraction (XRPD) pattern with peaks found in Table 2 as measured using Cu Kα.radiation.

In some embodiments of Compound 1 freebase Type B, the crystalline form has an X-ray powder diffraction (XRPD) pattern with peaks at 6.8±0.2° 2θ, 7.8±0.2° 2θ, and 12.8±0.2° 2θ as measured using Cu Kα.radiation.

In some embodiments of Compound 1 freebase Type B, the X-ray powder diffraction (XRPD) pattern further comprises peaks at 13.6±0.2° 2θ, 19.2±0.2° 2θ, and 22.0±0.2° 2θ as measured using Cu Kα.radiation.

In some embodiments of Compound 1 freebase Type B, the crystalline form has an X-ray powder diffraction (XRPD) pattern with peaks at 6.8±0.2° 2θ, 7.8±0.2° 2θ, 12.8±0.2° 2θ, 13.6±0. [symbol] 2θ, 19.2±0. [symbol] 2θ, and 22.0±0.2° 2θ as measured using Cu Kα.radiation.

In some embodiments of Compound 1 freebase Type B, the crystalline form has an X-ray powder diffraction (XRPD) pattern with at least two peaks selected from 6.8±0.2° 2θ, 7.8±0.2° 2θ, 12.8±0. [symbol] 2θ, 13.6±0.2° 2θ, 19.2±0.2° 2θ, and 22.0±0.2° 2θ as measured using Cu Kα.radiation.

In some embodiments of Compound 1 freebase Type B, the crystalline form has an X-ray powder diffraction (XRPD) pattern with at least three peaks selected from 6.8±0.2° 2θ, 7.8±0.2° 2θ, 12.8±0.2° 2θ, 13.6±0.2° 2θ, 19.2±0.2° 2θ, and 22.0±0.2° 2θ as measured using Cu Kα.radiation.

In some embodiments of Compound 1 freebase Type B, the crystalline form has an X-ray powder diffraction (XRPD) pattern with at least four peaks selected from 6.8±0.2° 2θ, 7.8±0.2° 2θ, 12.8±0.2° 2θ, 13.6±0.2° 2θ, 19.2±0.2° 2θ, and 22.0±0.2° 2θ as measured using Cu Kα.radiation.

In some embodiments of Compound 1 freebase Type B, the crystalline form has an X-ray powder diffraction (XRPD) pattern with at least five peaks selected from 6.8±0.2° 2θ, 7.8±0.2° 2θ, 12.8±0.2° 2θ, 13.6±0.2° 2θ, 19.2±0.2° 2θ, and 22.0±0.2° 2θ as measured using Cu Kα.radiation.

In some embodiments of Compound 1 freebase Type B, the Differential Scanning calorimetry (DSC) thermogram has an exothermic peak having a peak temperature at about 109.4 [symbol] .

In some embodiments of Compound 1 freebase Type B, the Differential Scanning calorimetry (DSC) thermogram has an endothermic peak having a peak temperature at about 87.5 [symbol] .

In some embodiments of Compound 1 freebase Type B, the Differential Scanning calorimetry (DSC) thermogram has an endothermic peak having a peak temperature at about 103.8 [symbol] .

In some embodiments of Compound 1 freebase Type B, the Differential Scanning calorimetry (DSC) thermogram has an endothermic peak having a peak temperature at about 226.7 [symbol] .

In some embodiments of Compound 1 freebase Type B, the Thermogravimetric Thermal Analysis (TGA) thermogram is substantially the same as shown in FIG. 4.

In some embodiments of Compound 1 freebase Type B, the Thermogravimetric Thermal Analysis (TGA) thermogram exhibits a mass loss of about 12.78% from the onset of heating up to approximately 150 [symbol] .

In some embodiments of Compound 1 freebase Type B, the crystalline form is a hydrate.

TABLE 2
X-Ray Powder Diffraction peaks of freebase Type B

| Pos. [°2θ] | Rel. Int. [%] |
|---|---|
| 6.82 | 16.39 |
| 7.75 | 100.00 |
| 12.10 | 3.89 |
| 12.83 | 74.55 |
| 13.60 | 11.49 |
| 16.45 | 9.18 |
| 18.34 | 7.84 |
| 19.19 | 22.78 |
| 20.40 | 4.27 |
| 21.60 | 5.74 |
| 22.01 | 17.95 |
| 22.72 | 7.74 |
| 23.34 | 3.51 |
| 24.72 | 13.90 |
| 25.85 | 33.80 |
| 26.14 | 10.86 |
| 27.86 | 3.63 |
| 28.34 | 8.27 |
| 31.82 | 3.37 |
| 36.66 | 1.67 |

### Compound 1 Freebase Type C

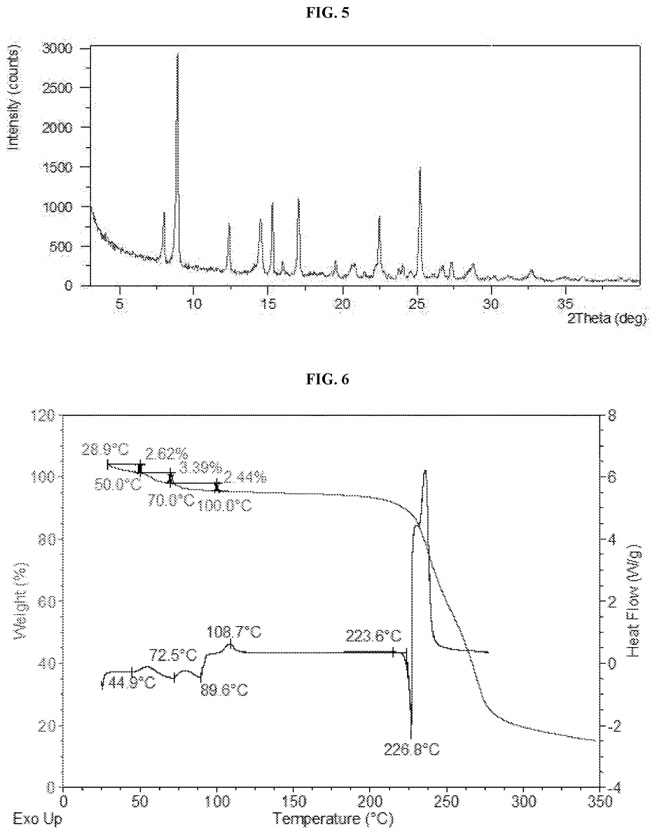

Disclosed herein is Compound 1 freebase Type C. In some embodiments, the crystalline form is Compound 1 freebase Type C characterized as having at least one of the following properties:
(a) an X-Ray powder diffraction (XRPD) pattern substantially the same as shown in FIG. 5 as measured using Cu Kα.radiation;
(b) an X-Ray powder diffraction (XRPD) pattern with peaks at 8.9±0.2° 2θ, 15.3±0.2° 2θ, and 17.0±0.2° 2θ as measured using Cu Kα.radiation;
(c) a Differential Scanning calorimetry (DSC) thermogram substantially the same as shown in FIG. 6;
(d) a Differential Scanning calorimetry (DSC) thermogram with an exothermic peak having a peak temperature at about 108.7 [symbol] ;
(e) a Differential Scanning calorimetry (DSC) thermogram with an endothermic peak having a peak temperature at about 44.9 [symbol] ;
(f) a Differential Scanning calorimetry (DSC) thermogram with an endothermic peak having a peak temperature at about 72.5 [symbol] ;
(g) a Differential Scanning calorimetry (DSC) thermogram with an endothermic peak having a peak temperature at about 89.6 [symbol] ;
(h) a Differential Scanning calorimetry (DSC) thermogram with an endothermic peak having a peak temperature at about 226.8 [symbol] ;
(i) a Thermogravimetric Thermal Analysis (TGA) thermogram substantially the same as shown in FIG. 6;
(j) a Thermogravimetric Thermal Analysis (TGA) thermogram exhibiting a mass loss of about 2.62% from the onset of heating up to approximately 50 [symbol] ;
(k) a Thermogravimetric Thermal Analysis (TGA) thermogram exhibiting a mass loss of about 3.39% from the onset of heating from 50.0 [symbol] to 70.0 [symbol] ;
(l) a Thermogravimetric Thermal Analysis (TGA) thermogram exhibiting a mass loss of about 2.44% from the onset of heating from 70.0 [symbol] to 100.0 [symbol] ; or
(m) combinations thereof.

In some embodiments, the crystalline form is Compound 1 freebase Type C characterized as having at least one of the following properties:
(a) an X-Ray powder diffraction (XRPD) pattern substantially the same as shown in FIG. 5 as measured using Cu Kα.radiation;
(b) an X-Ray powder diffraction (XRPD) pattern with peaks at 8.9±0.2° 2θ, 15.3±0.2° 2θ, and 17.0±0.2° 2θ as measured using Cu Kα.radiation;
(c) a Differential Scanning calorimetry (DSC) thermogram substantially the same as shown in FIG. 6;
(d) a Thermogravimetric Thermal Analysis (TGA) thermogram substantially the same as shown in FIG. 6; or
(e) combinations thereof.

In some embodiments, the crystalline form is Compound 1 freebase Type C characterized as having at least one of the following properties:
(a) an X-Ray powder diffraction (XRPD) pattern with peaks at 8.9±0.2° 2θ, 15.3±0.2° 2θ, and 17.0±0.2° 2θ as measured using Cu Kα.radiation;
(b) a Differential Scanning calorimetry (DSC) thermogram with an exothermic peak having a peak temperature at about 108.7 [symbol] ;
(c) a Differential Scanning calorimetry (DSC) thermogram with an endothermic peak having a peak temperature at about 44.9 [symbol] ;
(d) a Differential Scanning calorimetry (DSC) thermogram with an endothermic peak having a peak temperature at about 72.5 [symbol] ;
(e) a Differential Scanning calorimetry (DSC) thermogram with an endothermic peak having a peak temperature at about 89.6 [symbol] ;
(f) a Differential Scanning calorimetry (DSC) thermogram with an endothermic peak having a peak temperature at about 226.8 [symbol] ;
(g) a Thermogravimetric Thermal Analysis (TGA) thermogram exhibiting a mass loss of about 2.62% from the onset of heating up to approximately 50 [symbol] ;
(h) a Thermogravimetric Thermal Analysis (TGA) thermogram exhibiting a mass loss of about 3.39% from the onset of heating from 50.0 [symbol] to 70.0 [symbol] ;
(i) a Thermogravimetric Thermal Analysis (TGA) thermogram exhibiting a mass loss of about 2.44% from the onset of heating from 70.0 [symbol] to 100.0 [symbol] ; or
(j) combinations thereof.

In some embodiments of Compound 1 freebase Type C, the crystalline form has an X-ray powder diffraction (XRPD) pattern substantially the same as shown in FIG. 5 as measured using Cu Kα.radiation.

In some embodiments of Compound 1 freebase Type C, the crystalline form has an X-ray powder diffraction (XRPD) pattern with peaks found in Table 3 as measured using Cu Kα.radiation.

In some embodiments of Compound 1 freebase Type C, the crystalline form has an X-ray powder diffraction (XRPD) pattern with peaks at 8.9±0.2° 2θ, 15.3±0.2° 2θ, and 17.0±0.2° 2θ as measured using Cu Kα.radiation.

In some embodiments of Compound 1 freebase Type C, the X-ray powder diffraction (XRPD) pattern further comprises peaks at 8.0±0.2° 2θ, 12.4±0.2° 2θ, 14.5±0.2° 2θ as measured using Cu Kα.radiation.

In some embodiments of Compound 1 freebase Type C, the X-ray powder diffraction (XRPD) pattern further comprises peaks at 22.5±0.2° 2θ and 25.2±0.2° 2θ as measured using Cu Kα.radiation.

In some embodiments of Compound 1 freebase Type C, the crystalline form has an X-ray powder diffraction (XRPD) pattern with peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 2θ, and 25.2±0.2° 2θ as measured using Cu Kα.radiation.

In some embodiments of Compound 1 freebase Type C, the crystalline form has an X-ray powder diffraction (XRPD) pattern with at least two peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 2θ, and 25.2±0.2° 2θ as measured using Cu Kα.radiation.

In some embodiments of Compound 1 freebase Type C, the crystalline form has an X-ray powder diffraction (XRPD) pattern with at least three peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 2θ, and 25.2±0.2° 2θ as measured using Cu Kα.radiation.

In some embodiments of Compound 1 freebase Type C, the crystalline form has an X-ray powder diffraction (XRPD) pattern with at least four peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 2θ, and 25.2±0.2° 2θ as measured using Cu Kα.radiation.

In some embodiments of Compound 1 freebase Type C, the crystalline form has an X-ray powder diffraction (XRPD) pattern with at least five peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 2θ, and 25.2±0.2° 2θ as measured using Cu Kα.radiation.

In some embodiments of Compound 1 freebase Type C, the crystalline form has an X-ray powder diffraction (XRPD) pattern with at least six peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 2θ, and 25.2±0.2° 2θ as measured using Cu Kα.radiation.

In some embodiments of Compound 1 freebase Type C, the crystalline form has an X-ray powder diffraction (XRPD) pattern with at least seven peaks at 8.0±0.2° 2θ, 8.9±0.2° 2θ, 12.4±0.2° 2θ, 14.5±0.2° 2θ, 15.3±0.2° 2θ, 17.0±0.2° 2θ, 22.5±0.2° 20, and 25.2±0.2° 2θ as measured using Cu Kα.radiation.

In some embodiments, the Differential Scanning calorimetry (DSC) thermogram is substantially the same as shown in FIG. 6.

In some embodiments of Compound 1 freebase Type C, the Differential Scanning calorimetry (DSC) thermogram has an exothermic peak having a peak temperature at about 108.7 [symbol] .

In some embodiments of Compound 1 freebase Type C, the Differential Scanning calorimetry (DSC) thermogram has an endothermic peak having a peak temperature at about 44.9 [symbol] .

In some embodiments of Compound 1 freebase Type C, the Differential Scanning calorimetry (DSC) thermogram has an endothermic peak having a peak temperature at about 72.5 [symbol] .

In some embodiments of Compound 1 freebase Type C, the Differential Scanning calorimetry (DSC) thermogram has an endothermic peak having a peak temperature at about 89.6 [symbol] .

In some embodiments of Compound 1 freebase Type C, the Differential Scanning calorimetry (DSC) thermogram has an endothermic peak having a peak temperature at about 226.8 [symbol] .

In some embodiments of Compound 1 freebase Type C, the Thermogravimetric Thermal Analysis (TGA) thermogram is substantially the same as shown in FIG. 6.

In some embodiments of Compound 1 freebase Type C, the Thermogravimetric Thermal Analysis (TGA) thermogram exhibits a mass loss of about 2.62% from the onset of heating up to approximately 50 [symbol] .

In some embodiments of Compound 1 freebase Type C, the Thermogravimetric Thermal Analysis (TGA) thermogram exhibits a mass loss of about 3.39% from the onset of heating from 50.0 [symbol] to 70.0 [symbol] .

In some embodiments of Compound 1 freebase Type C, the Thermogravimetric Thermal Analysis (TGA) thermogram exhibits a mass loss of about 2.44% from the onset of heating from 70.0 [symbol] to 100.0 [symbol] .

In some embodiments of Compound 1 freebase Type C, the crystalline form is a hydrate.

TABLE 3
X-Ray Powder Diffraction peaks of freebase Type C

| Pos. [°2θ] | Rel. Int. [%] |
|---|---|
| 7.99 | 23.64 |
| 8.90 | 100.00 |
| 12.38 | 22.41 |
| 14.50 | 25.63 |
| 15.28 | 33.71 |
| 15.98 | 4.96 |
| 17.03 | 36.53 |
| 19.52 | 7.56 |
| 20.81 | 6.27 |
| 21.46 | 2.81 |
| 22.47 | 29.83 |
| 24.05 | 6.03 |
| 24.57 | 3.67 |
| 25.20 | 51.54 |
| 26.58 | 5.53 |
| 27.35 | 7.74 |
| 28.81 | 7.26 |
| 31.19 | 1.73 |
| 32.71 | 4.76 |
| 34.95 | 1.85 |
| 36.14 | 2.26 |

### Compound 1 HCl Salt Type A

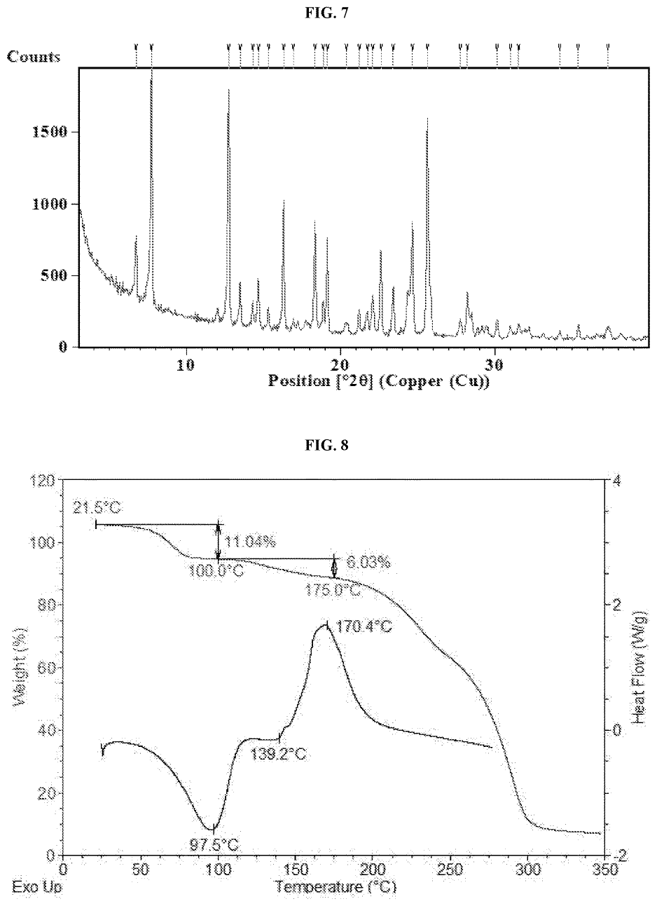

Disclosed herein is Compound 1 HCl salt Type A. In some embodiments, the crystalline form is Compound 1 HCl salt Type A characterized as having at least one of the following properties:
(a) an X-Ray powder diffraction (XRPD) pattern substantially the same as shown in FIG. 7 as measured using Cu Kα.radiation;
(b) an X-Ray powder diffraction (XRPD) pattern with peaks at 7.8±0.2° 2θ, 12.8±0.2° 2θ, and 25.6±0.2° 2θ as measured using Cu Kα.radiation;
(c) a Differential Scanning calorimetry (DSC) thermogram substantially the same as shown in FIG. 8;
(d) a Differential Scanning calorimetry (DSC) thermogram with an exothermic peak having a peak temperature at about 170.4 [symbol] ;
(e) a Differential Scanning calorimetry (DSC) thermogram with an endothermic peak having a peak temperature at about 97.5 [symbol] ;
(f) a Differential Scanning calorimetry (DSC) thermogram with an endothermic peak having a peak temperature at about 139.2 [symbol] ;
(g) a Thermogravimetric Thermal Analysis (TGA) thermogram substantially the same as shown in FIG. 8;
(h) a Thermogravimetric Thermal Analysis (TGA) thermogram exhibiting a mass loss of about 11.04% from the onset of heating up to approximately 100 [symbol] ;
(i) a Thermogravimetric Thermal Analysis (TGA) thermogram exhibiting a mass loss of about 6.03% from the onset of heating from 100 [symbol] to 175 [symbol] ; or
(j) combinations thereof.

In some embodiments, the crystalline form is Compound 1 HCl salt Type A characterized as having at least one of the following properties:
(a) an X-Ray powder diffraction (XRPD) pattern substantially the same as shown in FIG. 7 as measured using Cu Kα.radiation;
(b) an X-Ray powder diffraction (XRPD) pattern with peaks at 7.8±0.2° 2θ, 12.8=0.2° 2θ, and 25.6±0.2° 2θ as measured using Cu Kα.radiation;
(c) a Differential Scanning calorimetry (DSC) thermogram substantially the same as shown in FIG. 8;
(d) a Thermogravimetric Thermal Analysis (TGA) thermogram substantially the same as shown in FIG. 8; or
(e) combinations thereof.

In some embodiments, the crystalline form is Compound 1 HCl salt Type A characterized as having at least one of the following properties:
(a) an X-Ray powder diffraction (XRPD) pattern with peaks at 7.8±0.2° 2θ, 12.8±0.2° 2θ, and 25.6±0.2° 2θ as measured using Cu Kα.radiation;
(b) a Differential Scanning calorimetry (DSC) thermogram with an exothermic peak having a peak temperature at about 170.4 [symbol] ;
(c) a Differential Scanning calorimetry (DSC) thermogram with an endothermic peak having a peak temperature at about 97.5 [symbol] ;
(d) a Differential Scanning calorimetry (DSC) thermogram with an endothermic peak having a peak temperature at about 139.2 [symbol] ;
(e) a Thermogravimetric Thermal Analysis (TGA) thermogram exhibiting a mass loss of about 11.04% from the onset of heating up to approximately 100 [symbol] ;
(f) a Thermogravimetric Thermal Analysis (TGA) thermogram exhibiting a mass loss of about 6.03% from the onset of heating from 100 [symbol] to 175 [symbol] ; or
(g) combinations thereof.

In some embodiments of Compound 1 HCl salt Type A, the crystalline form has an X-ray powder diffraction (XRPD) pattern substantially the same as shown in FIG. 7 as measured using Cu Kα.radiation.

In some embodiments of Compound 1 HCl salt Type A, the crystalline form has an X-ray powder diffraction (XRPD) pattern with peaks found in Table 4 as measured using Cu Kα.radiation.

In some embodiments of Compound 1 HCl salt Type A, the crystalline form has an X-ray powder diffraction (XRPD) pattern with peaks at 7.8±0.2° 2θ, 12.8±0.2° 2θ, and 25.6±0.2° 2θ as measured using Cu Kα.radiation.

In some embodiments of Compound 1 HCl salt Type A, the X-ray powder diffraction (XRPD) pattern further comprises peaks at 6.8±0.2° 2θ, 16.3±0.2° 2θ, and 18.3±0.2° 2θ as measured using Cu Kα.radiation.

In some embodiments of Compound 1 HCl salt Type A, the X-ray powder diffraction (XRPD) pattern further comprises peaks at 19.1±0.2° 2θ, 22.6±0.2° 2θ, and 24.7±0.2° 20 as measured using Cu Kα.radiation.

In some embodiments of Compound 1 HCl salt Type A, the crystalline form has an X-ray powder diffraction (XRPD) pattern with peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least two peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least three peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least four peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least five peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least six peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least seven peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the crystalline form has an X-ray powder diffraction (XRPD) pattern with at least eight peaks at 6.8±0.2° 2θ, 7.8±0.2° 2θ, 12.8±0.2° 2θ, 16.3±0.2° 2θ, 18.3±0.2° 2θ, 19.1±0.2° 2θ, 22.6±0.2° 2θ, 24.7±0.2° 2θ, and 25.6±0.2° 2θ as measured using Cu Kα.radiation.

In some embodiments of Compound 1 HCl salt Type A, the Differential Scanning calorimetry (DSC) thermogram is substantially the same as shown in FIG. 8.

In some embodiments of Compound 1 HCl salt Type A, the Differential Scanning calorimetry (DSC) thermogram has an exothermic peak having a peak temperature at about 170.4 [symbol] .

In some embodiments of Compound 1 HCl salt Type A, the Differential Scanning calorimetry (DSC) thermogram has an endothermic peak having a peak temperature at about 97.5 [symbol] .

In some embodiments of Compound 1 HCl salt Type A, the Differential Scanning calorimetry (DSC) thermogram has an endothermic peak having a peak temperature at about 139.2 [symbol] .

In some embodiments of Compound 1 HCl salt Type A, the Thermogravimetric Thermal Analysis (TGA) thermogram is substantially the same as shown in FIG. 8.

In some embodiments of Compound 1 HCl salt Type A, the Thermogravimetric Thermal Analysis (TGA) thermogram exhibits a mass loss of about 11.04% from the onset of heating up to approximately 100 [symbol] .

In some embodiments of Compound 1 HCl salt Type A, the Thermogravimetric Thermal Analysis (TGA) thermogram exhibits a mass loss of about 6.03% from the onset of heating from 100 [symbol] to 175 [symbol] .

In some embodiments of Compound 1 HCl salt Type A, the molar ratio of HCl to Compound 1 freebase is about 0.6:1.

TABLE 4
X-Ray Powder Diffraction peaks of HCl salt Type A

| Pos. [°2θ] | Rel. Int. [%] |
|---|---|
| 6.75 | 27.15 |
| 7.75 | 98.28 |
| 12.75 | 100.00 |
| 13.49 | 18.17 |
| 14.31 | 10.88 |
| 14.67 | 19.78 |
| 15.32 | 8.38 |
| 16.30 | 52.61 |
| 16.92 | 3.47 |
| 18.34 | 46.05 |
| 18.88 | 12.87 |
| 19.14 | 38.41 |
| 20.38 | 3.84 |
| 21.21 | 8.86 |
| 21.75 | 9.83 |
| 22.08 | 16.25 |
| 22.61 | 34.91 |
| 23.42 | 20.23 |
| 24.65 | 46.29 |
| 25.63 | 89.72 |
| 27.76 | 6.88 |
| 28.21 | 17.44 |
| 30.14 | 7.14 |
| 30.99 | 5.15 |
| 31.55 | 5.85 |
| 34.19 | 2.65 |
| 35.39 | 5.54 |
| 37.32 | 4.98 |

### Compound 1 HCl Salt Type B

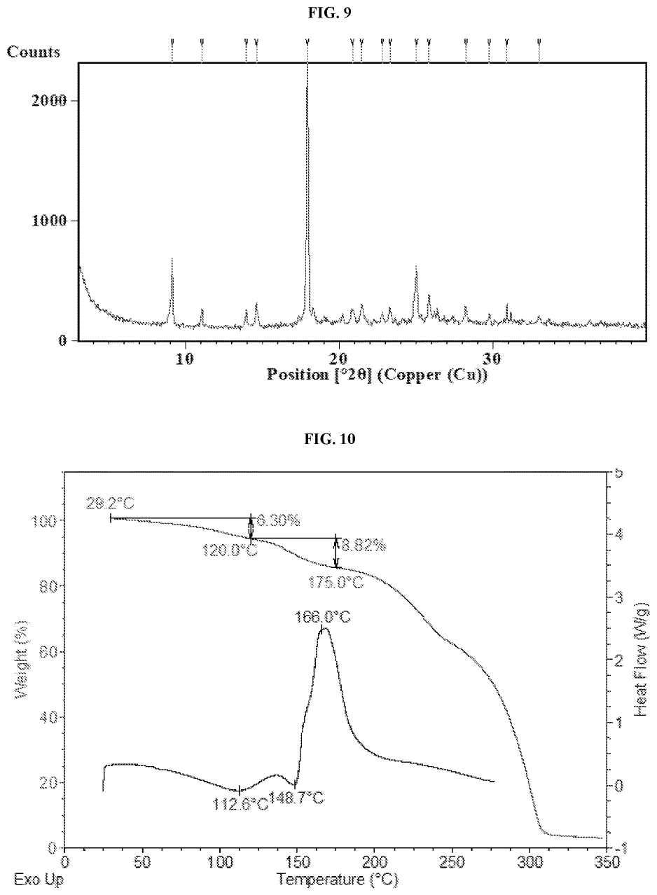

Disclosed herein is Compound 1 HCl salt Type B. In some embodiments, the crystalline form is Compound 1 HCl salt Type B characterized as having at least one of the following properties:
(a) an X-Ray powder diffraction (XRPD) pattern substantially the same as shown in FIG. 9 as measured using Cu Kα.radiation;
(b) an X-Ray powder diffraction (XRPD) pattern with peaks at 9.1±0. [symbol] 2θ, 14.6±0.2° 2θ, and 17.9±0.2° 2θ as measured using Cu Kα.radiation;
(c) a Differential Scanning calorimetry (DSC) thermogram substantially the same as shown in FIG. 10;
(d) a Differential Scanning calorimetry (DSC) thermogram with an exothermic peak having a peak temperature at about 166.0 [symbol] ;
(e) a Differential Scanning calorimetry (DSC) thermogram with an endothermic peak having a peak temperature at about 112.6 [symbol] ;
(f) a Differential Scanning calorimetry (DSC) thermogram with an endothermic peak having a peak temperature at about 148.7 [symbol] ;
(g) a Thermogravimetric Thermal Analysis (TGA) thermogram substantially the same as shown in FIG. 10;
(h) a Thermogravimetric Thermal Analysis (TGA) thermogram exhibiting a mass loss of about 6.30% from the onset of heating up to approximately 120 [symbol] ;
(i) a Thermogravimetric Thermal Analysis (TGA) thermogram exhibiting a mass loss of about 8.82% from the onset of heating from 120 [symbol] to 175 [symbol] ; or
(j) combinations thereof.

In some embodiments, the crystalline form is Compound 1 HCl salt Type B characterized as having at least one of the following properties:
(a) an X-Ray powder diffraction (XRPD) pattern substantially the same as shown in FIG. 9 as measured using Cu Kα.radiation;
(b) an X-Ray powder diffraction (XRPD) pattern with peaks at 9.1±0.2° 2θ, 14.6±0.2° 2θ, and 17.9±0.2° 2θ as measured using Cu Kα.radiation;
(c) a Differential Scanning calorimetry (DSC) thermogram substantially the same as shown in FIG. 10;
(d) a Thermogravimetric Thermal Analysis (TGA) thermogram substantially the same as shown in FIG. 10; or
(e) combinations thereof.

In some embodiments, the crystalline form is Compound 1 HCl salt Type B characterized as having at least one of the following properties:
(a) an X-Ray powder diffraction (XRPD) pattern with peaks at 9.1±0.2° 2θ, 14.6±0.2° 2θ, and 17.9±0.2° 2θ as measured using Cu Kα.radiation;
(b) a Differential Scanning calorimetry (DSC) thermogram with an exothermic peak having a peak temperature at about 166.0 [symbol] ;
(c) a Differential Scanning calorimetry (DSC) thermogram with an endothermic peak having a peak temperature at about 112.6 [symbol] ;
(d) a Differential Scanning calorimetry (DSC) thermogram with an endothermic peak having a peak temperature at about 148.7 [symbol] ;
(e) a Thermogravimetric Thermal Analysis (TGA) thermogram exhibiting a mass loss of about 6.30% from the onset of heating up to approximately 120 [symbol] ;
(f) a Thermogravimetric Thermal Analysis (TGA) thermogram exhibiting a mass loss of about 8.82% from the onset of heating from 120 [symbol] to 175 [symbol] ; or
(g) combinations thereof.

In some embodiments of Compound 1 HCl salt Type B, the crystalline form has an X-ray powder diffraction (XRPD) pattern substantially the same as shown in FIG. 9 as measured using Cu Kα.radiation.

In some embodiments of Compound 1 HCl salt Type B, the crystalline form has an X-ray powder diffraction (XRPD) pattern with peaks found in Table 5 as measured using Cu Kα.radiation.

In some embodiments of Compound 1 HCl salt Type B, the crystalline form has an X-ray powder diffraction (XRPD) pattern with peaks at 9.1±0.2° 2θ, 14.6±0.2° 2θ, and 17.9±0.2° 2θ as measured using Cu Kα.radiation.

In some embodiments of Compound 1 HCl salt Type B, the X-ray powder diffraction (XRPD) pattern further comprises peaks at 11.1±0.2° 2θ, 14.0±0.2° 2θ, and 25.0±0.2° 2θ as measured using Cu Kα.radiation.

In some embodiments of Compound 1 HCl salt Type B, the crystalline form has an X-ray powder diffraction (XRPD) pattern with peaks at 9.1±0.2° 2θ, 11.1±0.2° 2θ, 14.0±0.2° 2θ, 14.6±0.2° 2θ, 17.9±0.2° 2θ, and 25.0±0.2° 2θ as measured using Cu Kα.radiation.

In some embodiments of Compound 1 HCl salt Type B, the crystalline form has an X-ray powder diffraction (XRPD) pattern with at least two peaks at 9.1±0.2° 2θ, 11.1±0.2° 2θ, 14.0±0.2° 2θ, 14.6±0.2° 2θ, 17.9±0.2° 2θ, and 25.0±0.2° 2θ as measured using Cu Kα.radiation.

In some embodiments of Compound 1 HCl salt Type B, the crystalline form has an X-ray powder diffraction (XRPD) pattern with at least three peaks at 9.1±0.2° 2θ, 11.1±0.2° 2θ, 14.0±0.2° 2θ, 14.6±0.2° 2θ, 17.9±0.2° 2θ, and 25.0±0.2° 2θ as measured using Cu Kα.radiation.

In some embodiments of Compound 1 HCl salt Type B, the crystalline form has an X-ray powder diffraction (XRPD) pattern with at least four peaks at 9.1±0.2° 2θ, 11.1±0.2° 2θ, 14.0±0.2° 2θ, 14.6±0.2° 2θ, 17.9±0.2° 2θ, and 25.0±0.2° 2θ as measured using Cu Kα.radiation.

In some embodiments of Compound 1 HCl salt Type B, the crystalline form has an X-ray powder diffraction (XRPD) pattern with at least five peaks at 9.1±0.2° 2θ, 11.1±0.2° 2θ, 14.0±0.2° 2θ, 14.6±0.2° 2θ, 17.9±0.2° 2θ, and 25.0±0.2° 2θ as measured using Cu Kα.radiation.

In some embodiments of Compound 1 HCl salt Type B, the Differential Scanning calorimetry (DSC) thermogram is substantially the same as shown in FIG. 10.

In some embodiments of Compound 1 HCl salt Type B, the Differential Scanning calorimetry (DSC) thermogram has an exothermic peak having a peak temperature at about 166.0 [symbol] .

In some embodiments of Compound 1 HCl salt Type B, the Differential Scanning calorimetry (DSC) thermogram has an endothermic peak having a peak temperature at about 112.6 [symbol] .

In some embodiments of Compound 1 HCl salt Type B, the Differential Scanning calorimetry (DSC) thermogram has an endothermic peak having a peak temperature at about 148.7 [symbol] .

In some embodiments of Compound 1 HCl salt Type B, the Thermogravimetric Thermal Analysis (TGA) thermogram is substantially the same as shown in FIG. 10.

In some embodiments of Compound 1 HCl salt Type B, the Thermogravimetric Thermal Analysis (TGA) thermogram exhibits a mass loss of about 6.30% from the onset of heating up to approximately 120 [symbol] .

In some embodiments of Compound 1 HCl salt Type B, the Thermogravimetric Thermal Analysis (TGA) thermogram exhibits a mass loss of about 8.82% from the onset of heating from 120 [symbol] to 175 [symbol] .

In some embodiments of Compound 1 HCl salt Type B, the molar ratio of HCl to Compound 1 freebase is about 1:1.

TABLE 5
X-Ray Powder Diffraction peaks of HCl salt Type B

| Pos. [°2θ] | Rel. Int. [%] |
|---|---|
| 9.14 | 25.51 |
| 11.08 | 7.16 |
| 13.97 | 7.19 |
| 14.62 | 9.83 |
| 17.94 | 100.00 |
| 20.87 | 5.66 |
| 21.47 | 8.02 |
| 22.81 | 4.38 |
| 23.30 | 6.43 |
| 25.03 | 21.93 |
| 25.84 | 11.23 |
| 28.25 | 6.54 |
| 29.76 | 3.56 |
| 30.92 | 8.35 |
| 33.01 | 2.69 |

### Compound 1 HCl Salt Type C

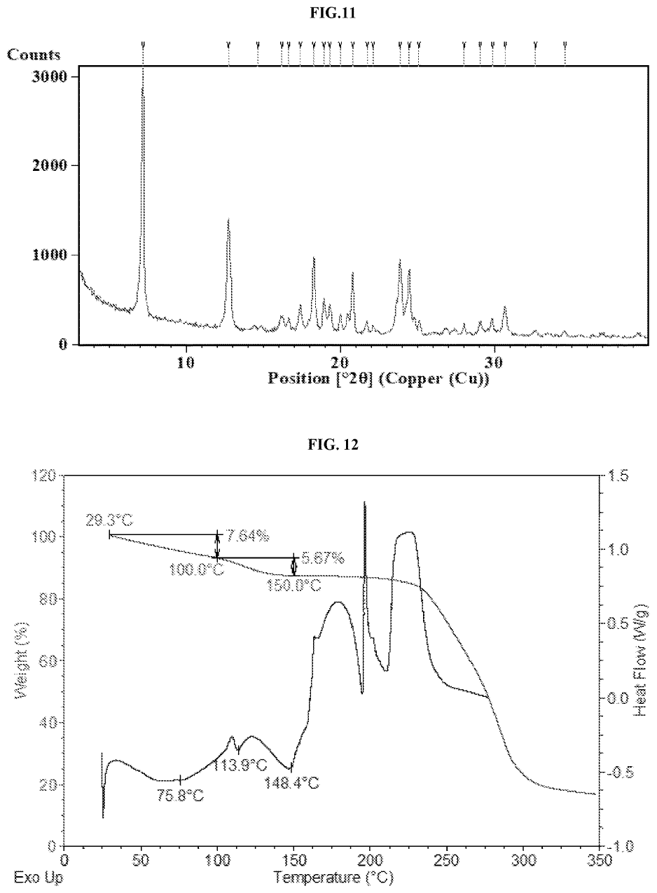

Disclosed herein is Compound 1 HCl salt Type C. In some embodiments, the crystalline form is Compound 1 HCl salt Type C characterized as having at least one of the following properties:
(a) an X-Ray powder diffraction (XRPD) pattern substantially the same as shown in FIG. 11 as measured using Cu Kα.radiation;
(b) an X-Ray powder diffraction (XRPD) pattern with peaks at 7.2±0.2° 2θ, 12.7±0.2° 2θ, and 18.3±0.2° 2θ as measured using Cu Kα.radiation;
(c) a Differential Scanning calorimetry (DSC) thermogram substantially the same as shown in FIG. 12;
(d) a Differential Scanning calorimetry (DSC) thermogram with an endothermic peak having a peak temperature at about 75.8 [symbol] ;
(e) a Differential Scanning calorimetry (DSC) thermogram with an endothermic peak having a peak temperature at about 113.9 [symbol] ;
(f) a Differential Scanning calorimetry (DSC) thermogram with an endothermic peak having a peak temperature at about 148.4 [symbol] ;

(g) a Thermogravimetric Thermal Analysis (TGA) thermogram substantially the same as shown in FIG. 12;
(h) a Thermogravimetric Thermal Analysis (TGA) thermogram exhibiting a mass loss of about 7.64% from the onset of heating up to approximately 100 [symbol] ;
(i) a Thermogravimetric Thermal Analysis (TGA) thermogram exhibiting a mass loss of about 5.67% from the onset of heating from 100 [symbol] to 150 [symbol] ; or
(j) combinations thereof.

In some embodiments, the crystalline form is Compound 1 HCl salt Type C characterized as having at least one of the following properties:
(a) an X-Ray powder diffraction (XRPD) pattern substantially the same as shown in FIG. 11 as measured using Cu Kα.radiation;
(b) an X-Ray powder diffraction (XRPD) pattern with peaks at 7.2±0.2° 2θ, 12.7±0.2° 2θ, and 18.3±0.2° 2θ as measured using Cu Kα.radiation;
(c) a Differential Scanning calorimetry (DSC) thermogram substantially the same as shown in FIG. 12;
(d) a Thermogravimetric Thermal Analysis (TGA) thermogram substantially the same as shown in FIG. 12; or
(e) combinations thereof.

In some embodiments, the crystalline form is Compound 1 HCl salt Type C characterized as having at least one of the following properties:
(a) an X-Ray powder diffraction (XRPD) pattern with peaks at 7.2±0.2° 2θ, 12.7±0.2° 2θ, and 18.3±0.2° 2θ as measured using Cu Kα.radiation;
(b) a Differential Scanning calorimetry (DSC) thermogram with an endothermic peak having a peak temperature at about 75.8 [symbol] ;
(c) a Differential Scanning calorimetry (DSC) thermogram with an endothermic peak having a peak temperature at about 113.9 [symbol] ;
(d) a Differential Scanning calorimetry (DSC) thermogram with an endothermic peak having a peak temperature at about 148.4 [symbol] ;
(e) a Thermogravimetric Thermal Analysis (TGA) thermogram exhibiting a mass loss of about 7.64% from the onset of heating up to approximately 100 [symbol] ;
(f) a Thermogravimetric Thermal Analysis (TGA) thermogram exhibiting a mass loss of about 5.67% from the onset of heating from 100 [symbol] to 150 [symbol] ; or
(g) combinations thereof.

In some embodiments of Compound 1 HCl salt Type C, the crystalline form has an X-ray powder diffraction (XRPD) pattern substantially the same as shown in FIG. 11 as measured using Cu Kα.radiation.

In some embodiments of Compound 1 HCl salt Type C, the crystalline form has an X-ray powder diffraction (XRPD) pattern with peaks found in Table 6 as measured using Cu Kα.radiation.

In some embodiments of Compound 1 HCl salt Type C, the crystalline form has an X-ray powder diffraction (XRPD) pattern with peaks at 7.2±0.2° 2θ, 12.7±0.2° 2θ, and 18.3±0.2° 2θ as measured using Cu Kα.radiation.

In some embodiments of Compound 1 HCl salt Type C, the X-ray powder diffraction (XRPD) pattern further comprises peaks at 17.4±0.2° 2θ, 20.8±0.2° 2θ, and 23.9±0.2° 2θ as measured using Cu Kα.radiation.

In some embodiments of Compound 1 HCl salt Type C, the crystalline form has an X-ray powder diffraction (XRPD) pattern with peaks at 7.2±0.2° 2θ, 12.7±0.2° 2θ, 17.4±0.2° 2θ, 18.3±0.2° 2θ, 20.8±0.2° 2θ, and 23.9±0.2° 2θ as measured using Cu Kα.radiation.

In some embodiments of Compound 1 HCl salt Type C, the crystalline form has an X-ray powder diffraction (XRPD) pattern with at least two peaks at 7.2±0.2° 2θ, 12.7±0.2° 2θ, 17.4±0.2° 2θ, 18.3±0.2° 2θ, 20.8±0.2° 2θ, and 23.9±0.2° 2θ as measured using Cu Kα.radiation.

In some embodiments of Compound 1 HCl salt Type C, the crystalline form has an X-ray powder diffraction (XRPD) pattern with at least three peaks at 7.2±0.2° 2θ, 12.7±0.2° 2θ, 17.4±0.2° 2θ, 18.3±0.2° 2θ, 20.8±0.2° 2θ, and 23.9±0.2° 2θ as measured using Cu Kα.radiation.

In some embodiments of Compound 1 HCl salt Type C, the crystalline form has an X-ray powder diffraction (XRPD) pattern with at least four peaks at 7.2±0.2° 2θ, 12.7±0.2° 2θ, 17.4±0.2° 2θ, 18.3±0.2° 2θ, 20.8±0.2° 2θ, and 23.9±0.2° 2θ as measured using Cu Kα.radiation.

In some embodiments of Compound 1 HCl salt Type C, the crystalline form has an X-ray powder diffraction (XRPD) pattern with at least five peaks at 7.2±0.2° 2θ, 12.7±0.2° 2θ, 17.4±0.2° 2θ, 18.3±0.2° 2θ, 20.8±0.2° 2θ, and 23.9±0.2° 2θ as measured using Cu Kα.radiation.

In some embodiments of Compound 1 HCl salt Type C, the Differential Scanning calorimetry (DSC) thermogram is substantially the same as shown in FIG. 12.

In some embodiments of Compound 1 HCl salt Type C, the Differential Scanning calorimetry (DSC) thermogram has an endothermic peak having a peak temperature at about 75.8 [symbol] .

In some embodiments of Compound 1 HCl salt Type C, the Differential Scanning calorimetry (DSC) thermogram has an endothermic peak having a peak temperature at about 113.9 [symbol] .

In some embodiments of Compound 1 HCl salt Type C, the Differential Scanning calorimetry (DSC) thermogram has an endothermic peak having a peak temperature at about 148.4 [symbol] .

In some embodiments of Compound 1 HCl salt Type C, the Thermogravimetric Thermal Analysis (TGA) thermogram is substantially the same as shown in FIG. 12.

In some embodiments of Compound 1 HCl salt Type C, the Thermogravimetric Thermal Analysis (TGA) thermogram exhibits a mass loss of about 7.64% from the onset of heating up to approximately 100 [symbol] .

In some embodiments of Compound 1 HCl salt Type C, the Thermogravimetric Thermal Analysis (TGA) thermogram exhibits a mass loss of about 5.67% from the onset of heating from 100 [symbol] to 150 [symbol] .

In some embodiments of Compound 1 HCl salt Type C, the molar ratio of HCl to Compound 1 freebase is about 0.7:1.

TABLE 6
X-Ray Powder Diffraction peaks of HCl salt Type C

| Pos. [°2θ] | Rel. Int. [%] |
|---|---|
| 7.20 | 100.00 |
| 12.73 | 44.04 |
| 14.64 | 0.97 |
| 16.21 | 5.67 |
| 16.64 | 4.91 |
| 17.39 | 10.84 |
| 18.28 | 29.77 |
| 18.92 | 12.19 |
| 19.32 | 11.00 |
| 19.99 | 6.73 |
| 20.80 | 23.81 |
| 21.73 | 4.90 |
| 22.13 | 2.98 |
| 23.87 | 29.92 |
| 24.47 | 25.62 |
| 25.10 | 5.27 |
| 28.03 | 4.14 |
| 29.08 | 5.16 |
| 29.89 | 6.33 |
| 30.70 | 11.65 |
| 32.65 | 2.01 |
| 34.56 | 2.07 |

### Compound 1 Sulfate Salt Type A

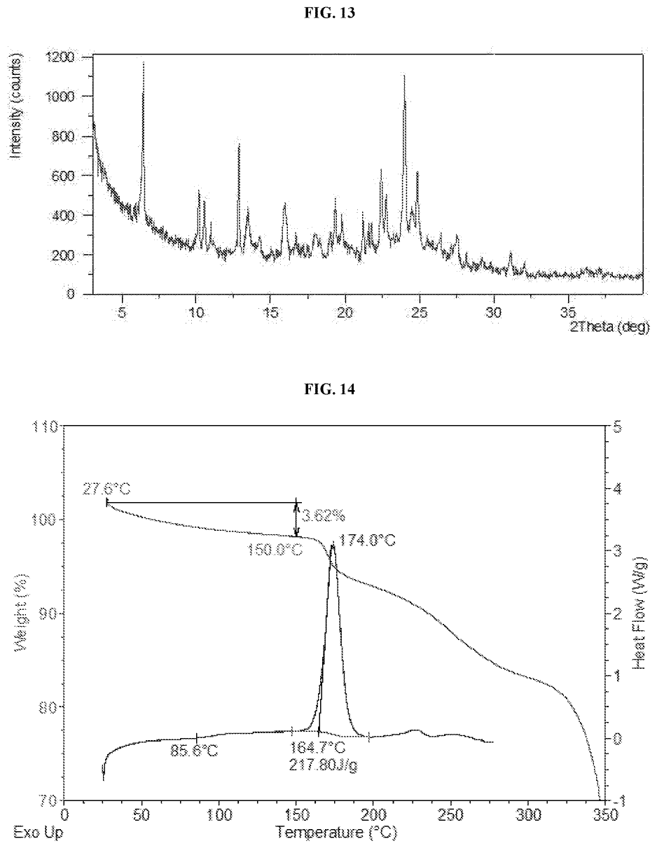

Disclosed herein is Compound 1 Sulfate salt Type A. In some embodiments, the crystalline form is Compound 1 Sulfate salt Type A characterized as having at least one of the following properties:
(a) an X-Ray powder diffraction (XRPD) pattern substantially the same as shown in FIG. 13 as measured using Cu Kα.radiation;
(b) an X-Ray powder diffraction (XRPD) pattern with peaks at 6.5±0.2° 2θ, 12.9±0.2° 2θ, and 24.0±0.2° 2θ as measured using Cu Kα.radiation;
(c) a Differential Scanning calorimetry (DSC) thermogram substantially the same as shown in FIG. 14;
(d) a Differential Scanning calorimetry (DSC) thermogram with an exothermic peak having a peak temperature at about 174.0 [symbol] ;
(e) a Differential Scanning calorimetry (DSC) thermogram with an endothermic peak having a peak temperature at about 85.6 [symbol] ;
(f) a Thermogravimetric Thermal Analysis (TGA) thermogram substantially the same as shown in FIG. 14;

(g) a Thermogravimetric Thermal Analysis (TGA) thermogram exhibiting a mass loss of about 3.62% from the onset of heating up to approximately 150 [symbol] ; or
(h) combinations thereof.

In some embodiments, the crystalline form is Compound 1 Sulfate salt Type A characterized as having at least one of the following properties:
(a) an X-Ray powder diffraction (XRPD) pattern substantially the same as shown in FIG. 13 as measured using Cu Kα.radiation;
(b) an X-Ray powder diffraction (XRPD) pattern with peaks at 6.5±0.2° 2θ, 12.9±0.2° 2θ, and 24.0±0.2° 2θ as measured using Cu Kα.radiation;
(c) a Differential Scanning calorimetry (DSC) thermogram substantially the same as shown in FIG. 14;
(d) a Thermogravimetric Thermal Analysis (TGA) thermogram substantially the same as shown in FIG. 14; or
(e) combinations thereof.

In some embodiments, the crystalline form is Compound 1 Sulfate salt Type A characterized as having at least one of the following properties:
(a) an X-Ray powder diffraction (XRPD) pattern with peaks at 6.5±0.2° 2θ, 12.9±0.2° 2θ, and 24.0±0.2° 2θ as measured using Cu Kα.radiation;
(b) a Differential Scanning calorimetry (DSC) thermogram with an exothermic peak having a peak temperature at about 174.0 [symbol] ;
(c) a Differential Scanning calorimetry (DSC) thermogram with an endothermic peak having a peak temperature at about 85.6 [symbol] ;
(d) a Thermogravimetric Thermal Analysis (TGA) thermogram exhibiting a mass loss of about 3.62% from the onset of heating up to approximately 150 [symbol] ; or
(e) combinations thereof.

In some embodiments of Compound 1 Sulfate salt Type A, the crystalline form has an X-ray powder diffraction (XRPD) pattern substantially the same as shown in FIG. 13 as measured using Cu Kα.radiation.

In some embodiments of Compound 1 Sulfate salt Type A, the crystalline form has an X-ray powder diffraction (XRPD) pattern with peaks found in Table 7 as measured using Cu Kα.radiation.

In some embodiments of Compound 1 Sulfate salt Type A, the crystalline form has an X-ray powder diffraction (XRPD) pattern with peaks at 6.5±0.2° 2θ, 12.9±0.2° 2θ, and 24.0±0.2° 2θ as measured using Cu Kα.radiation.

In some embodiments of Compound 1 Sulfate salt Type A, the X-ray powder diffraction (XRPD) pattern further comprises peaks at 10.2±0.2° 2θ, 13.5±0.2° 2θ, 15.9±0.2° 2θ, and 19.4±0.2° 2θ as measured using Cu Kα.radiation.

In some embodiments of Compound 1 Sulfate salt Type A, the X-ray powder diffraction (XRPD) pattern further comprises peaks at 21.2±0.2° 2θ, 22.4±0.2° 2θ, and 24.9±0.2° 20 as measured using Cu Kα.radiation.

In some embodiments of Compound 1 Sulfate salt Type A, the crystalline form has an X-ray powder diffraction (XRPD) pattern with peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least two peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least three peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least four peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least five peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least six peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least seven peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least eight peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the crystalline form has an X-ray powder diffraction (XRPD) pattern with at least nine peaks at 6.5±0.2° 2θ, 10.2±0.2° 2θ, 12.9±0.2° 2θ, 13.5±0.2° 2θ, 15.9±0.2° 2θ, 19.4±0.2° 2θ, 21.2±0.2° 2θ, 22.4±0.2° 2θ, 24.0±0.2° 2θ, and 24.9±0.2° 2θ as measured using Cu Kα.radiation.

In some embodiments of Compound 1 Sulfate salt Type A, the Differential Scanning calorimetry (DSC) thermogram is substantially the same as shown in FIG. 14.

In some embodiments of Compound 1 Sulfate salt Type A, the Differential Scanning calorimetry (DSC) thermogram has an exothermic peak having a peak temperature at about 174.0 [symbol] .

In some embodiments of Compound 1 Sulfate salt Type A, the Differential Scanning calorimetry (DSC) thermogram has an endothermic peak having a peak temperature at about 85.6 [symbol] .

In some embodiments of Compound 1 Sulfate salt Type A, the Thermogravimetric Thermal Analysis (TGA) thermogram is substantially the same as shown in FIG. 14.

In some embodiments of Compound 1 Sulfate salt Type A, the Thermogravimetric Thermal Analysis (TGA) thermogram exhibits a mass loss of about 3.62% from the onset of heating up to approximately 150 [symbol] .

In some embodiments of Compound 1 Sulfate salt Type A, the molar ratio of sulfuric acid to Compound 1 freebase is about 1.2:1.

TABLE 7
X-Ray Powder Diffraction peaks of sulfate Type A

| Pos. [°2θ] | Rel. Int. [%] |
|---|---|
| 6.45 | 84.05 |
| 10.18 | 31.80 |
| 10.55 | 27.23 |
| 12.87 | 59.62 |
| 13.45 | 23.46 |
| 15.87 | 26.48 |
| 18.03 | 12.41 |
| 19.35 | 30.26 |
| 19.78 | 18.57 |
| 21.22 | 23.61 |
| 22.43 | 44.69 |
| 22.73 | 31.20 |
| 24.00 | 100.00 |
| 24.86 | 47.15 |
| 26.36 | 13.47 |
| 27.53 | 17.01 |
| 31.12 | 11.09 |

### Compound 1 Maleate Salt Type A

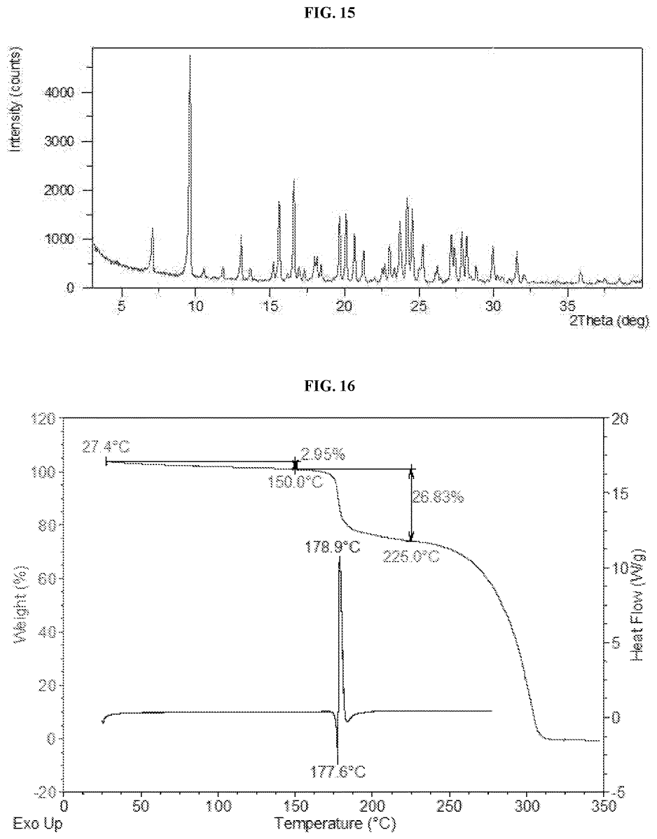

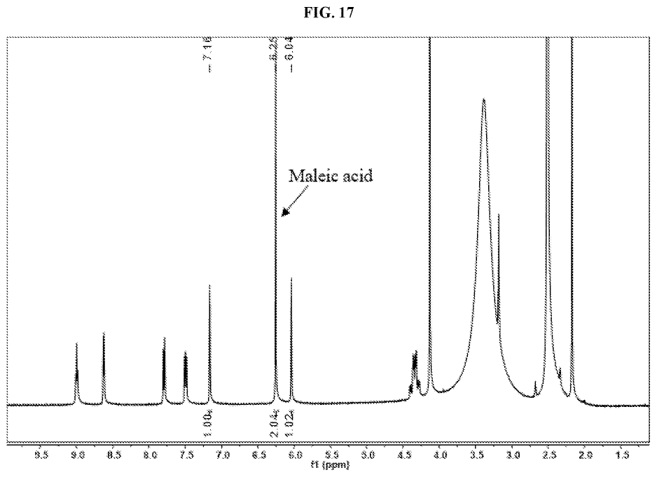

Disclosed herein is Compound 1 Maleate salt Type A. In some embodiments, the crystalline form is Compound 1 Maleate salt Type A characterized as having at least one of the following properties:
(a) an X-Ray powder diffraction (XRPD) pattern substantially the same as shown in FIG. 15 as measured using Cu Kα.radiation;
(b) an X-Ray powder diffraction (XRPD) pattern with peaks at 9.6±0.2° 2θ, 15.6±0.2° 2θ, and 16.6±0.2° 2θ as measured using Cu Kα.radiation;
(c) a Differential Scanning calorimetry (DSC) thermogram substantially the same as shown in FIG. 16;
(d) a Differential Scanning calorimetry (DSC) thermogram with an exothermic peak having a peak temperature at about 178.9 [symbol] ;
(e) a Differential Scanning calorimetry (DSC) thermogram with an endothermic peak having a peak temperature at about 177.6 [symbol] ;
(f) a Thermogravimetric Thermal Analysis (TGA) thermogram substantially the same as shown in FIG. 16;
(g) a Thermogravimetric Thermal Analysis (TGA) thermogram exhibiting a mass loss of about 2.95% from the onset of heating up to approximately 150.0 [symbol] ;
(h) a Thermogravimetric Thermal Analysis (TGA) thermogram exhibiting a mass loss of about 26.83% from the onset of heating from 150 [symbol] to 225 [symbol] ; or
(i) combinations thereof.

In some embodiments, the crystalline form is Compound 1 Maleate salt Type A characterized as having at least one of the following properties:
(a) an X-Ray powder diffraction (XRPD) pattern substantially the same as shown in FIG. 15 as measured using Cu Kα.radiation;
(b) an X-Ray powder diffraction (XRPD) pattern with peaks at 9.6±0. [symbol] 2θ, 15.6±0.2° 2θ, and 16.6±0.2° 2θ as measured using Cu Kα.radiation;
(c) a Differential Scanning calorimetry (DSC) thermogram substantially the same as shown in FIG. 16;
(d) a Thermogravimetric Thermal Analysis (TGA) thermogram substantially the same as shown in FIG. 16; or
(e) combinations thereof.

In some embodiments, the crystalline form is Compound 1 Maleate salt Type A characterized as having at least one of the following properties:
(a) an X-Ray powder diffraction (XRPD) pattern with peaks at 9.6±0.2° 2θ, 15.6±0.2° 2θ, and 16.6±0.2° 2θ as measured using Cu Kα.radiation;
(b) a Differential Scanning calorimetry (DSC) thermogram with an exothermic peak having a peak temperature at about 178.9 [symbol] ;
(c) a Differential Scanning calorimetry (DSC) thermogram with an endothermic peak having a peak temperature at about 177.6 [symbol] ;
(d) a Thermogravimetric Thermal Analysis (TGA) thermogram exhibiting a mass loss of about 2.95% from the onset of heating up to approximately 150.0 [symbol] ;
(e) a Thermogravimetric Thermal Analysis (TGA) thermogram exhibiting a mass loss of about 26.83% from the onset of heating from 150 [symbol] to 225 [symbol] ; or
(f) combinations thereof.

In some embodiments of Compound 1 Maleate salt Type A, the crystalline form has an X-ray powder diffraction (XRPD) pattern substantially the same as shown in FIG. 15 as measured using Cu Kα.radiation.

In some embodiments of Compound 1 Maleate salt Type A, the crystalline form has an X-ray powder diffraction (XRPD) pattern with peaks found in Table 8 as measured using Cu Kα.radiation. In some embodiments of Compound 1 Maleate salt Type A, the crystalline form has an X-ray powder diffraction (XRPD) pattern with 1, 2, 3, 4, 5, or 6 peaks found in Table 8 as measured using Cu Kα.radiation.

In some embodiments of Compound 1 Maleate salt Type A, the crystalline form has an X-ray powder diffraction (XRPD) pattern with peaks at 9.6±0.2° 2θ, 15.6±0.2° 2θ, and 16.6±0.2° 2θ as measured using Cu Kα.radiation.

In some embodiments of Compound 1 Maleate salt Type A, the X-ray powder diffraction (XRPD) pattern further comprises peaks at 13.0±0.2° 2θ, 19.7±0.2° 2θ, and 20.6±0.2° 2θ as measured using Cu Kα.radiation.

In some embodiments of Compound 1 Maleate salt Type A, the X-ray powder diffraction (XRPD) pattern further comprises peaks at 7.1±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least two peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least three peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least four peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 20, and 24.5±0.2° 2θ as measured using Cu Kα.radiation.

In some embodiments of Compound 1 Maleate salt Type A, the crystalline form has an X-ray powder diffraction (XRPD) pattern with at least five peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least six peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least seven peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least eight peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the crystalline form has an X-ray powder diffraction (XRPD) pattern with at least nine peaks at 7.1±0.2° 2θ, 9.6±0.2° 2θ, 13.0±0.2° 2θ, 15.6±0.2° 2θ, 16.6±0.2° 2θ, 19.7±0.2° 2θ, 20.6±0.2° 2θ, 23.0±0.2° 2θ, 23.7±0.2° 2θ, and 24.5±0.2° 2θ as measured using Cu Kα.radiation.

In some embodiments of Compound 1 Maleate salt Type A, the Differential Scanning calorimetry (DSC) thermogram is substantially the same as shown in FIG. 16.

In some embodiments of Compound 1 Maleate salt Type A, the Differential Scanning calorimetry (DSC) thermogram has an exothermic peak having a peak temperature at about 178.9 [symbol] .

In some embodiments of Compound 1 Maleate salt Type A, the Differential Scanning calorimetry (DSC) thermogram has an endothermic peak having a peak temperature at about 177.6 [symbol] .

In some embodiments of Compound 1 Maleate salt Type A, the Thermogravimetric Thermal Analysis (TGA) thermogram is substantially the same as shown in FIG. 16.

In some embodiments of Compound 1 Maleate salt Type A, the Thermogravimetric Thermal Analysis (TGA) thermogram exhibits a mass loss of about 2.95% from the onset of heating up to approximately 150.0 [symbol] .

In some embodiments of Compound 1 Maleate salt Type A, the Thermogravimetric Thermal Analysis (TGA) thermogram exhibits a mass loss of about 26.83% from the onset of heating from 150 [symbol] to 225 [symbol] .

In some embodiments of Compound 1 Maleate salt Type A, the crystalline form is anhydrous.

In some embodiments of Compound 1 Maleate salt Type A, the crystalline form is non-hygroscopic.

In some embodiments of Compound 1 Maleate salt Type A, the molar ratio of maleic acid to Compound 1 freebase is about 1:1.

TABLE 8
X-Ray Powder Diffraction peaks of maleate Type A

| Pos. [°2θ] | Rel. Int. [%] |
|---|---|
| 7.07 | 10.67 |
| 9.59 | 100.00 |
| 10.51 | 3.93 |
| 11.79 | 4.93 |
| 13.03 | 16.43 |
| 13.65 | 5.44 |
| 15.18 | 7.42 |
| 15.58 | 38.04 |
| 16.14 | 2.66 |
| 16.57 | 47.86 |
| 16.93 | 4.91 |
| 17.26 | 4.41 |
| 17.93 | 9.57 |
| 18.11 | 9.34 |
| 18.38 | 5.70 |
| 19.21 | 1.86 |
| 19.65 | 25.84 |
| 20.10 | 27.63 |
| 20.64 | 21.44 |
| 21.25 | 9.22 |
| 21.96 | 1.64 |
| 22.48 | 5.34 |
| 22.66 | 5.43 |
| 23.01 | 14.22 |
| 23.35 | 6.19 |
| 23.68 | 23.14 |
| 24.17 | 33.94 |
| 24.52 | 36.16 |
| 24.97 | 5.08 |
| 25.23 | 17.77 |
| 26.20 | 6.51 |
| 26.44 | 1.98 |
| 27.13 | 21.38 |
| 27.38 | 12.38 |
| 27.89 | 17.55 |
| 28.17 | 19.96 |
| 28.81 | 5.79 |
| 29.93 | 14.11 |
| 30.58 | 2.73 |
| 31.54 | 11.53 |
| 32.04 | 2.69 |
| 33.99 | 0.93 |
| 34.33 | 1.27 |
| 34.93 | 0.75 |
| 35.83 | 4.51 |
| 36.97 | 1.72 |
| 37.47 | 2.11 |
| 38.43 | 2.08 |
| 39.29 | 0.59 |

### Compound 1 Phosphate Salt Type A

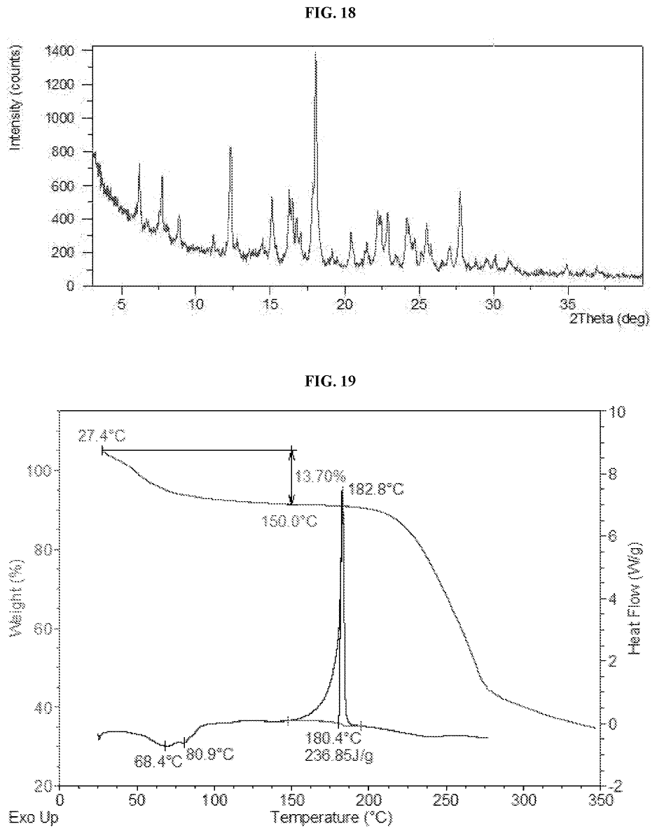

Disclosed herein is Compound 1 Phosphate salt Type A. In some embodiments, the crystalline form is Compound 1 Phosphate salt Type A characterized as having at least one of the following properties:
(a) an X-Ray powder diffraction (XRPD) pattern substantially the same as shown in FIG. 18 as measured using Cu Kα.radiation;
(b) an X-Ray powder diffraction (XRPD) pattern with peaks at 7.7±0.2° 2θ, 12.3±0.2° 2θ, and 18.1±0.2° 2θ as measured using Cu Kα.radiation;
(c) a Differential Scanning calorimetry (DSC) thermogram substantially the same as shown in FIG. 19;
(d) a Differential Scanning calorimetry (DSC) thermogram with an exothermic peak having a peak temperature at about 182.8 [symbol] ;
(e) a Differential Scanning calorimetry (DSC) thermogram with an endothermic peak having a peak temperature at about 68.4 [symbol] ;
(f) a Differential Scanning calorimetry (DSC) thermogram with an endothermic peak having a peak temperature at about 80.9 [symbol] ;
(g) a Thermogravimetric Thermal Analysis (TGA) thermogram substantially the same as shown in FIG. 19;
(h) a Thermogravimetric Thermal Analysis (TGA) thermogram exhibiting a mass loss of about 13.70% from the onset of heating up to approximately 150.0° C.; or
(i) combinations thereof.

In some embodiments, the crystalline form is Compound 1 Phosphate salt Type A characterized as having at least one of the following properties:
(a) an X-Ray powder diffraction (XRPD) pattern substantially the same as shown in FIG. 18 as measured using Cu Kα.radiation;
(b) an X-Ray powder diffraction (XRPD) pattern with peaks at 7.7±0.2° 2θ, 12.3±0.2° 2θ, and 18.1±0.2° 2θ as measured using Cu Kα.radiation;
(c) a Differential Scanning calorimetry (DSC) thermogram substantially the same as shown in FIG. 19;
(d) a Thermogravimetric Thermal Analysis (TGA) thermogram substantially the same as shown in FIG. 19; or
(e) combinations thereof.

In some embodiments, the crystalline form is Compound 1 Phosphate salt Type A characterized as having at least one of the following properties:
(a) an X-Ray powder diffraction (XRPD) pattern with peaks at 7.7±0.2° 2θ, 12.3±0.2° 2θ, and 18.1±0.2° 2θ as measured using Cu Kα.radiation;
(b) a Differential Scanning calorimetry (DSC) thermogram with an exothermic peak having a peak temperature at about 182.8 [symbol] ;
(c) a Differential Scanning calorimetry (DSC) thermogram with an endothermic peak having a peak temperature at about 68.4 [symbol] ;
(d) a Differential Scanning calorimetry (DSC) thermogram with an endothermic peak having a peak temperature at about 80.9 [symbol] ;
(e) a Thermogravimetric Thermal Analysis (TGA) thermogram exhibiting a mass loss of about 13.70% from the onset of heating up to approximately 150.0 [symbol] ; or
(f) combinations thereof.

In some embodiments of Compound 1 Phosphate salt Type A, the crystalline form has an X-ray powder diffraction (XRPD) pattern substantially the same as shown in FIG. 18 as measured using Cu Kα.radiation.

In some embodiments of Compound 1 Phosphate salt Type A, the crystalline form has an X-ray powder diffraction (XRPD) pattern with peaks found in Table 9 as measured using Cu Kα.radiation.

In some embodiments of Compound 1 Phosphate salt Type A, the crystalline form has an X-ray powder diffraction (XRPD) pattern with peaks at 7.7±0.2° 2θ, 12.3±0.2° 2θ, and 18.1±0.2° 2θ as measured using Cu Kα.radiation.

In some embodiments of Compound 1 Phosphate salt Type A, the X-ray powder diffraction (XRPD) pattern further comprises peaks at 6.2±0.2° 2θ and 15.1±0.2° 2θ as measured using Cu Kα.radiation.

In some embodiments of Compound 1 Phosphate salt Type A, the X-ray powder diffraction (XRPD) pattern further comprises peaks at 16.3±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with at least two peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with at least three peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with at least four peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with at least five peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with at least six peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the crystalline form has an X-ray powder diffraction (XRPD) pattern with at least seven peaks at 6.2±0.2° 2θ, 7.7±0.2° 2θ, 12.3±0.2° 2θ, 15.1±0.2° 2θ, 16.3±0.2° 2θ, 18.1±0.2° 2θ, 22.2±0.2° 2θ, and 22.9±0.2° 2θ as measured using Cu Kα.radiation.

In some embodiments of Compound 1 Phosphate salt Type A, the Differential Scanning calorimetry (DSC) thermogram is substantially the same as shown in FIG. 19.

In some embodiments of Compound 1 Phosphate salt Type A, the Differential Scanning calorimetry (DSC) thermogram has an exothermic peak having a peak temperature at about 182.8 [symbol] .

In some embodiments of Compound 1 Phosphate salt Type A, the Differential Scanning calorimetry (DSC) thermogram has an endothermic peak having a peak temperature at about 68.4 [symbol] .

In some embodiments of Compound 1 Phosphate salt Type A, the Differential Scanning calorimetry (DSC) thermogram has an endothermic peak having a peak temperature at about 80.9 [symbol] .

In some embodiments of Compound 1 Phosphate salt Type A, the Thermogravimetric Thermal Analysis (TGA) thermogram is substantially the same as shown in FIG. 19.

In some embodiments, of Compound 1 Phosphate salt Type A the Thermogravimetric Thermal Analysis (TGA) thermogram exhibits a mass loss of about 13.70% from the onset of heating up to approximately 150.0 [symbol] .

In some embodiments of Compound 1 Phosphate salt Type A, the molar ratio of phosphoric acid to Compound 1 freebase is about 1.1:1.

TABLE 9
X-Ray Powder Diffraction peaks of Phosphate Type A

| Pos. [°2θ] | Rel. Int. [%] |
|---|---|
| 6.19 | 27.35 |
| 7.72 | 28.87 |
| 8.86 | 13.92 |
| 11.16 | 8.48 |
| 12.35 | 51.79 |
| 15.11 | 28.42 |
| 16.27 | 34.56 |
| 16.49 | 28.31 |
| 18.06 | 100.00 |
| 19.15 | 6.37 |
| 20.43 | 13.32 |
| 21.47 | 10.53 |
| 22.20 | 24.30 |
| 22.88 | 25.57 |
| 24.17 | 23.63 |
| 24.72 | 11.47 |
| 25.51 | 20.57 |
| 27.04 | 10.46 |
| 27.75 | 37.36 |
| 31.07 | 5.87 |
| 34.92 | 4.16 |

### Compound 1 Citrate Salt Type A

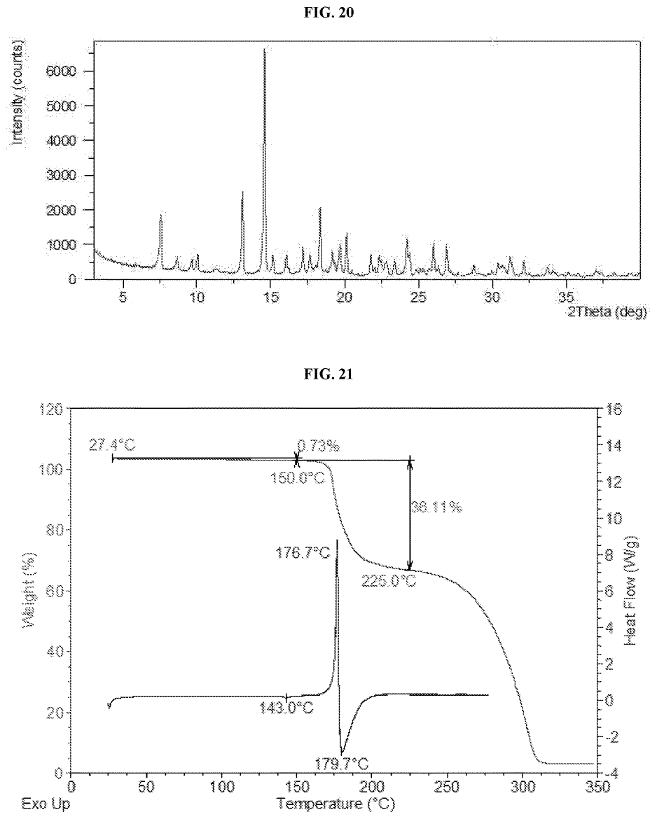

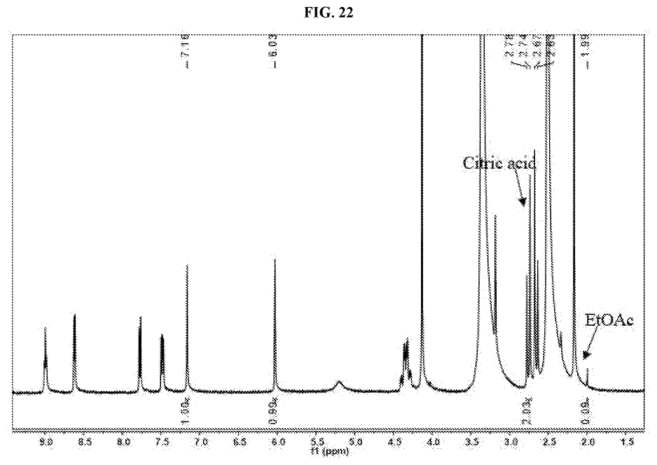

Disclosed herein is Compound 1 Citrate salt Type A. In some embodiments, the crystalline form is Compound 1 Citrate salt Type A characterized as having at least one of the following properties:
(a) an X-Ray powder diffraction (XRPD) pattern substantially the same as shown in FIG. 20 as measured using Cu Kα.radiation;
(b) an X-Ray powder diffraction (XRPD) pattern with peaks at 7.6±0.2° 2θ, 13.1±0.2° 2θ, and 14.6±0.2° 2θ as measured using Cu Kα.radiation;
(c) a Differential Scanning calorimetry (DSC) thermogram substantially the same as shown in FIG. 21;
(d) a Differential Scanning calorimetry (DSC) thermogram with an exothermic peak having a peak temperature at about 176.7 [symbol] ;
(e) a Differential Scanning calorimetry (DSC) thermogram with an endothermic peak having a peak temperature at about 143.0 [symbol] ;
(f) a Differential Scanning calorimetry (DSC) thermogram with an endothermic peak having a peak temperature at about 179.7 [symbol] ;
(g) a Thermogravimetric Thermal Analysis (TGA) thermogram substantially the same as shown in FIG. 21;
(h) a Thermogravimetric Thermal Analysis (TGA) thermogram exhibiting a mass loss of about 0.73% from the onset of heating up to approximately 150.0 [symbol] ;
(i) a Thermogravimetric Thermal Analysis (TGA) thermogram exhibiting a mass loss of about 36.11% from the onset of heating from 150 [symbol] to 225 [symbol] ; or
(j) combinations thereof.

In some embodiments, the crystalline form is Compound 1 Citrate salt Type A characterized as having at least one of the following properties:
(a) an X-Ray powder diffraction (XRPD) pattern substantially the same as shown in FIG. 20 as measured using Cu Kα.radiation;
(b) an X-Ray powder diffraction (XRPD) pattern with peaks at 7.6±0.2° 2θ, 13.1±0.2° 2θ, and 14.6±0.2° 2θ as measured using Cu Kα.radiation;
(c) a Differential Scanning calorimetry (DSC) thermogram substantially the same as shown in FIG. 21;
(d) a Thermogravimetric Thermal Analysis (TGA) thermogram substantially the same as shown in FIG. 21; or
(e) combinations thereof.

In some embodiments, the crystalline form is Compound 1 Citrate salt Type A characterized as having at least one of the following properties:
(a) an X-Ray powder diffraction (XRPD) pattern with peaks at 7.6±0.2° 2θ, 13.1±0.2° 2θ, and 14.6±0.2° 2θ as measured using Cu Kα.radiation;
(b) a Differential Scanning calorimetry (DSC) thermogram with an exothermic peak having a peak temperature at about 176.7 [symbol] ;
(c) a Differential Scanning calorimetry (DSC) thermogram with an endothermic peak having a peak temperature at about 143.0 [symbol] ;
(d) a Differential Scanning calorimetry (DSC) thermogram with an endothermic peak having a peak temperature at about 179.7 [symbol] ;
(e) a Thermogravimetric Thermal Analysis (TGA) thermogram exhibiting a mass loss of about 0.73% from the onset of heating up to approximately 150.0 [symbol] ;
(f) a Thermogravimetric Thermal Analysis (TGA) thermogram exhibiting a mass loss of about 36.11% from the onset of heating from 150 [symbol] to 225 [symbol] ; or
(g) combinations thereof.

In some embodiments of Compound 1 Citrate salt Type A, the crystalline form has an X-ray powder diffraction (XRPD) pattern substantially the same as shown in FIG. 20 as measured using Cu Kα.radiation.

In some embodiments of Compound 1 Citrate salt Type A, the crystalline form has an X-ray powder diffraction (XRPD) pattern with peaks found in Table 10 as measured using Cu Kα.radiation.

In some embodiments of Compound 1 Citrate salt Type A, the crystalline form has an X-ray powder diffraction (XRPD) pattern with peaks at 7.6±0.2° 2θ, 13.1±0.2° 2θ, and 14.6±0.2° 2θ as measured using Cu Kα.radiation.

In some embodiments of Compound 1 Citrate salt Type A, the X-ray powder diffraction (XRPD) pattern further comprises peaks at 17.2±0.2° 2θ, 18.3±0.2° 2θ, and 20.1±0.2° 2θ as measured using Cu Kα.radiation.

In some embodiments of Compound 1 Citrate salt Type A, the crystalline form has an X-ray powder diffraction (XRPD) pattern with peaks at 7.6±0.2° 2θ, 13.1±0.2° 2θ, 14.6±0.2° 2θ, 17.2±0.2° 2θ, 18.3±0.2° 2θ, and 20.1±0.2° 2θ as measured using Cu Kα.radiation.

In some embodiments of Compound 1 Citrate salt Type A, the crystalline form has an X-ray powder diffraction (XRPD) pattern with at least two peaks at 7.6±0.2° 2θ, 13.1±0.2° 2θ, 14.6±0.2° 2θ, 17.2±0.2° 2θ, 18.3±0.2° 2θ, and 20.1±0.2° 2θ as measured using Cu Kα.radiation.

In some embodiments of Compound 1 Citrate salt Type A, the crystalline form has an X-ray powder diffraction (XRPD) pattern with at least three peaks at 7.6±0.2° 2θ, 13.1±0.2° 2θ, 14.6±0.2° 2θ, 17.2±0.2° 2θ, 18.3±0.2° 2θ, and 20.1±0.2° 2θ as measured using Cu Kα.radiation.

In some embodiments of Compound 1 Citrate salt Type A, the crystalline form has an X-ray powder diffraction (XRPD) pattern with at least four peaks at 7.6±0.2° 2θ, 13.1±0.2° 2θ, 14.6±0.2° 2θ, 17.2±0.2° 2θ, 18.3±0.2° 2θ, and 20.1±0.2° 2θ as measured using Cu Kα.radiation.

In some embodiments of Compound 1 Citrate salt Type A, the crystalline form has an X-ray powder diffraction (XRPD) pattern with at least five peaks at 7.6±0.2° 2θ, 13.1±0.2° 2θ, 14.6±0.2° 2θ, 17.2±0.2° 2θ, 18.3±0.2° 2θ, and 20.1±0.2° 2θ as measured using Cu Kα.radiation.

In some embodiments of Compound 1 Citrate salt Type A, the Differential Scanning calorimetry (DSC) thermogram is substantially the same as shown in FIG. 21.

In some embodiments of Compound 1 Citrate salt Type A, the Differential Scanning calorimetry (DSC) thermogram has an exothermic peak having a peak temperature at about 176.7 [symbol] .

In some embodiments of Compound 1 Citrate salt Type A, the Differential Scanning calorimetry (DSC) thermogram has an endothermic peak having a peak temperature at about 143.0 [symbol] .

In some embodiments of Compound 1 Citrate salt Type A, the Differential Scanning calorimetry (DSC) thermogram has an endothermic peak having a peak temperature at about 179.7 [symbol] .

In some embodiments of Compound 1 Citrate salt Type A, the Thermogravimetric Thermal Analysis (TGA) thermogram is substantially the same as shown in FIG. 21.

In some embodiments of Compound 1 Citrate salt Type A, the Thermogravimetric Thermal Analysis (TGA) thermogram exhibits a mass loss of about 0.73% from the onset of heating up to approximately 150.0 [symbol] .

In some embodiments of Compound 1 Citrate salt Type A, the Thermogravimetric Thermal Analysis (TGA) thermogram exhibits a mass loss of about 36.11% from the onset of heating from 150 [symbol] to 225 [symbol] .

In some embodiments of Compound 1 Citrate salt Type A, the molar ratio of citric acid to Compound 1 freebase is about 1:1.

TABLE 10
X-Ray Powder Diffraction peaks of Citrate Type A

| Pos. [°2θ] | Rel. Int. [%] |
|---|---|
| 7.56 | 15.52 |
| 8.65 | 6.35 |
| 9.64 | 4.21 |
| 10.04 | 5.60 |
| 11.28 | 1.19 |
| 11.88 | 0.36 |
| 13.09 | 24.09 |
| 14.58 | 100.00 |
| 15.14 | 7.22 |
| 16.05 | 5.84 |
| 17.17 | 10.68 |
| 17.66 | 5.51 |
| 18.35 | 18.99 |
| 18.69 | 3.92 |
| 19.16 | 6.34 |
| 19.33 | 4.69 |
| 19.70 | 9.63 |
| 20.13 | 12.25 |
| 20.48 | 1.36 |
| 21.77 | 6.43 |
| 22.03 | 4.77 |
| 22.32 | 7.76 |
| 22.85 | 4.49 |
| 23.38 | 3.82 |
| 24.20 | 12.83 |
| 24.40 | 7.04 |
| 24.86 | 2.03 |
| 25.35 | 2.79 |
| 25.75 | 3.02 |
| 26.01 | 7.38 |
| 26.30 | 2.69 |
| 26.89 | 10.18 |
| 27.17 | 1.28 |
| 28.74 | 2.41 |
| 29.97 | 1.70 |
| 30.36 | 3.45 |
| 30.63 | 2.91 |
| 31.15 | 3.93 |
| 32.11 | 3.90 |
| 33.70 | 2.16 |
| 34.06 | 1.86 |
| 35.09 | 0.93 |
| 35.79 | 0.70 |
| 37.05 | 1.44 |
| 38.26 | 0.69 |
| 39.33 | 0.17 |

### Compound 1 L-Malate Salt Type A

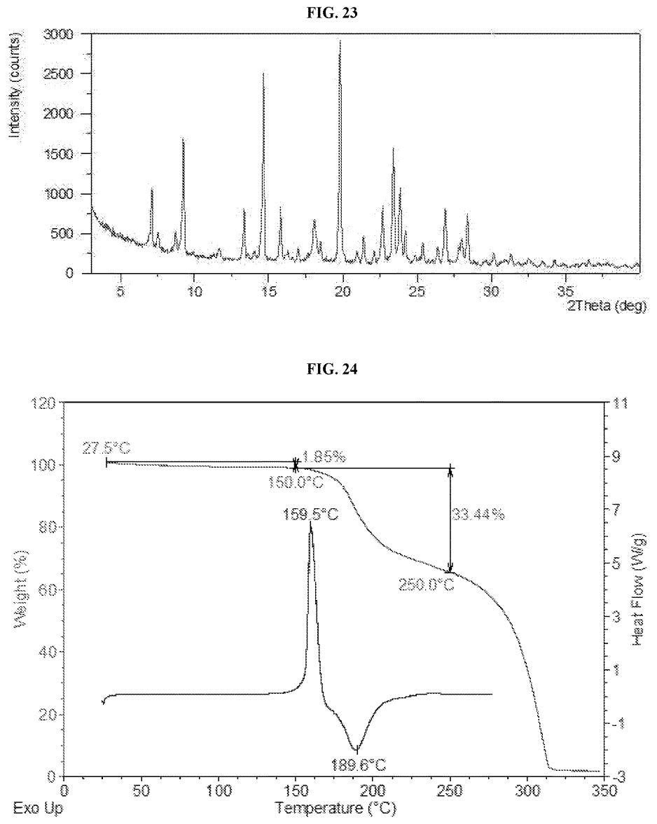

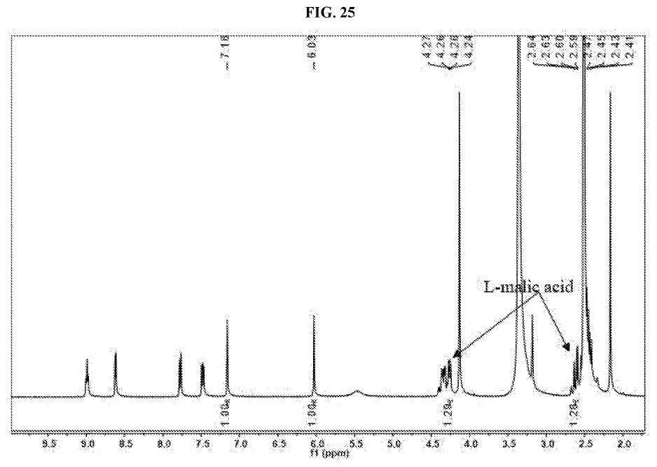

Disclosed herein is Compound 1 L-malate salt Type A. In some embodiments, the crystalline form is Compound 1 L-malate salt Type A characterized as having at least one of the following properties:
(a) an X-Ray powder diffraction (XRPD) pattern substantially the same as shown in FIG. 23 as measured using Cu Kα.radiation;
(b) an X-Ray powder diffraction (XRPD) pattern with peaks at 9.2±0.2° 2θ, 14.6±0.2° 2θ, and 19.8±0.2° 2θ as measured using Cu Kα.radiation;
(c) a Differential Scanning calorimetry (DSC) thermogram substantially the same as shown in FIG. 24;
(d) a Differential Scanning calorimetry (DSC) thermogram with an exothermic peak having a peak temperature at about 159.5 [symbol] ;
(e) a Differential Scanning calorimetry (DSC) thermogram with an endothermic peak having a peak temperature at about 189.6 [symbol] ;
(f) a Thermogravimetric Thermal Analysis (TGA) thermogram substantially the same as shown in FIG. 24;

(g) a Thermogravimetric Thermal Analysis (TGA) thermogram exhibiting a mass loss of about 1.85% from the onset of heating up to approximately 150.0 [symbol] ;
(h) a Thermogravimetric Thermal Analysis (TGA) thermogram exhibiting a mass loss of about 33.44% from the onset of heating from 150 [symbol] to 250 [symbol] ; or
(i) combinations thereof.

In some embodiments, the crystalline form is Compound 1 L-malate salt Type A characterized as having at least one of the following properties:
(a) an X-Ray powder diffraction (XRPD) pattern substantially the same as shown in FIG. 23 as measured using Cu Kα.radiation;
(b) an X-Ray powder diffraction (XRPD) pattern with peaks at 9.2±0. [symbol] 2θ, 14.6±0.2° 2θ, and 19.8±0.2° 2θ as measured using Cu Kα.radiation;
(c) a Differential Scanning calorimetry (DSC) thermogram substantially the same as shown in FIG. 24;
(d) a Thermogravimetric Thermal Analysis (TGA) thermogram substantially the same as shown in FIG. 24; or
(e) combinations thereof.

In some embodiments, the crystalline form is Compound 1 L-malate salt Type A characterized as having at least one of the following properties:
(a) an X-Ray powder diffraction (XRPD) pattern with peaks at 9.2±0.2° 2θ, 14.6±0.2° 2θ, and 19.8±0.2° 2θ as measured using Cu Kα.radiation;
(b) a Differential Scanning calorimetry (DSC) thermogram with an exothermic peak having a peak temperature at about 159.5 [symbol] ;
(c) a Differential Scanning calorimetry (DSC) thermogram with an endothermic peak having a peak temperature at about 189.6 [symbol] ;
(d) a Thermogravimetric Thermal Analysis (TGA) thermogram exhibiting a mass loss of about 1.85% from the onset of heating up to approximately 150.0 [symbol] ;
(e) a Thermogravimetric Thermal Analysis (TGA) thermogram exhibiting a mass loss of about 33.44% from the onset of heating from 150 [symbol] to 250 [symbol] ; or
(f) combinations thereof.

In some embodiments of Compound 1 L-malate salt Type A, the crystalline form has an X-ray powder diffraction (XRPD) pattern substantially the same as shown in FIG. 23 as measured using Cu Kα.radiation.

In some embodiments of Compound 1 L-malate salt Type A, the crystalline form has an X-ray powder diffraction (XRPD) pattern with peaks found in Table 11 as measured using Cu Kα.radiation.

In some embodiments of Compound 1 L-malate salt Type A, the crystalline form has an X-ray powder diffraction (XRPD) pattern with peaks at 9.2±0.2° 2θ, 14.6±0.2° 2θ, and 19.8±0.2° 2θ as measured using Cu Kα.radiation.

In some embodiments of Compound 1 L-malate salt Type A, the X-ray powder diffraction (XRPD) pattern further comprises peaks at 7.1±0.2° 2θ, 13.3±0.2° 2θ, and 15.5±0.2° 2θ as measured using Cu Kα.radiation.

In some embodiments of Compound 1 L-malate salt Type A, the X-ray powder diffraction (XRPD) pattern further comprises peaks at 18.1±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least two peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least three peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least four peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least five peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least six peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least seven peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the crystalline form has an X-ray powder diffraction (XRPD) pattern with at least eight peaks at 7.1±0.2° 2θ, 9.2±0.2° 2θ, 13.3±0.2° 2θ, 14.6±0.2° 2θ, 15.5±0.2° 2θ, 18.1±0.2° 2θ, 19.8±0.2° 2θ, 23.4±0.2° 2θ, and 26.9±0.2° 2θ as measured using Cu Kα.radiation.

In some embodiments of Compound 1 L-malate salt Type A, the Differential Scanning calorimetry (DSC) thermogram is substantially the same as shown in FIG. 24.

In some embodiments of Compound 1 L-malate salt Type A, the Differential Scanning calorimetry (DSC) thermogram has an exothermic peak having a peak temperature at about 159.5 [symbol] .

In some embodiments of Compound 1 L-malate salt Type A, the Differential Scanning calorimetry (DSC) thermogram has an endothermic peak having a peak temperature at about 189.6 [symbol] .

In some embodiments of Compound 1 L-malate salt Type A, the Thermogravimetric Thermal Analysis (TGA) thermogram is substantially the same as shown in FIG. 24.

In some embodiments of Compound 1 L-malate salt Type A, the Thermogravimetric Thermal Analysis (TGA) thermogram exhibits a mass loss of about 1.85% from the onset of heating up to approximately 150.0 [symbol] .

In some embodiments of Compound 1 L-malate salt Type A, the Thermogravimetric Thermal Analysis (TGA) thermogram exhibits a mass loss of about 33.44% from the onset of heating from 150 [symbol] to 250 [symbol] .

In some embodiments of Compound 1 L-malate salt Type A, the molar ratio of L-malic acid to Compound 1 freebase is about 1.3:1.

TABLE 11
X-Ray Powder Diffraction peaks of L-malate Type A

| Pos. [°2θ] | Rel. Int. [%] |
|---|---|
| 7.12 | 25.25 |
| 7.54 | 6.68 |
| 8.72 | 9.00 |
| 9.25 | 51.79 |
| 11.62 | 4.31 |
| 13.34 | 22.81 |
| 14.64 | 83.14 |
| 15.80 | 21.38 |
| 16.97 | 6.03 |
| 18.05 | 18.93 |
| 18.48 | 9.00 |
| 19.81 | 100.00 |
| 20.94 | 4.62 |
| 21.40 | 11.52 |
| 22.11 | 5.37 |
| 22.67 | 24.57 |
| 23.39 | 50.98 |
| 23.85 | 30.57 |
| 24.21 | 14.69 |
| 25.37 | 9.47 |
| 26.39 | 7.52 |
| 26.91 | 25.30 |
| 27.98 | 11.55 |
| 28.38 | 22.54 |
| 30.14 | 5.27 |
| 31.32 | 5.31 |
| 32.52 | 3.05 |
| 33.45 | 1.90 |
| 34.27 | 2.82 |
| 36.57 | 3.30 |
| 37.78 | 1.42 |

### Compound 1 Succinate Salt Type A

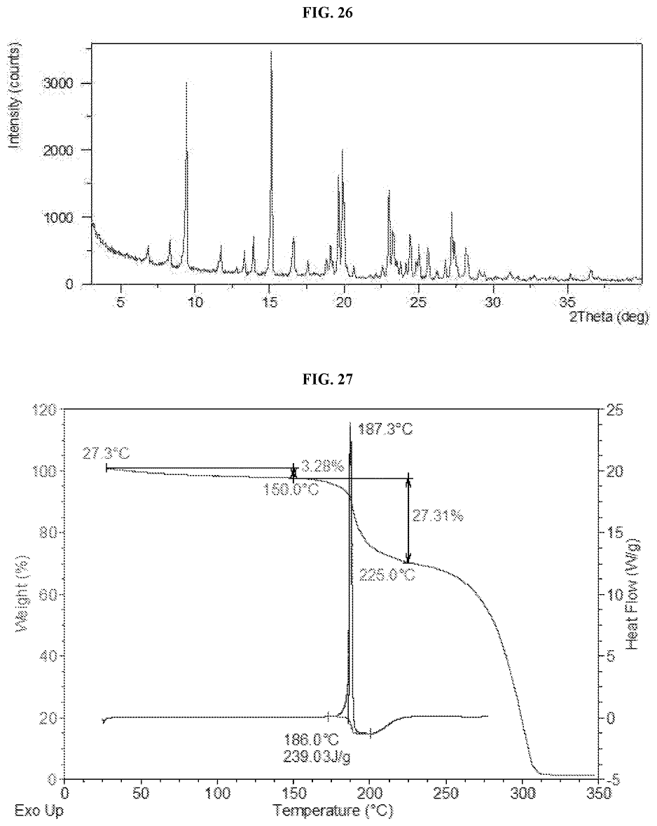

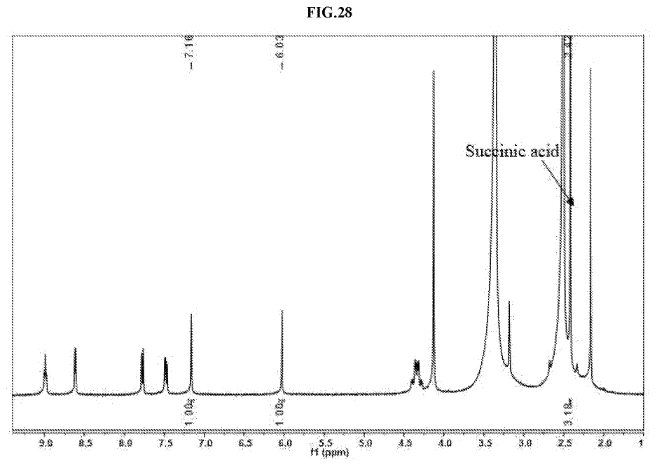

Disclosed herein is Compound 1 Succinate salt Type A. In some embodiments, the crystalline form is Compound 1 Succinate salt Type A characterized as having at least one of the following properties:
(a) an X-Ray powder diffraction (XRPD) pattern substantially the same as shown in FIG. 26 as measured using Cu Kα.radiation;
(b) an X-Ray powder diffraction (XRPD) pattern with peaks at 9.4±0.2° 2θ, 15.1±0.2° 2θ, and 19.9±0.2° 2θ as measured using Cu Kα.radiation;
(c) a Differential Scanning calorimetry (DSC) thermogram substantially the same as shown in FIG. 27;
(d) a Differential Scanning calorimetry (DSC) thermogram with an exothermic peak having a peak temperature at about 187.3 [symbol] ;
(e) a Thermogravimetric Thermal Analysis (TGA) thermogram substantially the same as shown in FIG. 27;
(f) a Thermogravimetric Thermal Analysis (TGA) thermogram exhibiting a mass loss of about 3.28% from the onset of heating up to approximately 150.0 [symbol] ;
(g) a Thermogravimetric Thermal Analysis (TGA) thermogram exhibiting a mass loss of about 27.31% from the onset of heating from 150 [symbol] to 225 [symbol] ; or
(h) combinations thereof.

In some embodiments, the crystalline form is Compound 1 Succinate salt Type A characterized as having at least one of the following properties:
(a) an X-Ray powder diffraction (XRPD) pattern substantially the same as shown in FIG. 26 as measured using Cu Kα.radiation;
(b) an X-Ray powder diffraction (XRPD) pattern with peaks at 9.4±0. [symbol] 2θ, 15.1±0.2° 2θ, and 19.9±0.2° 2θ as measured using Cu Kα.radiation;
(c) a Differential Scanning calorimetry (DSC) thermogram substantially the same as shown in FIG. 27;
(d) a Thermogravimetric Thermal Analysis (TGA) thermogram substantially the same as shown in FIG. 27; or
(e) combinations thereof.

In some embodiments, the crystalline form is Compound 1 Succinate salt Type A characterized as having at least one of the following properties:
(a) an X-Ray powder diffraction (XRPD) pattern with peaks at 9.4±0.2° 2θ, 15.1±0.2° 2θ, and 19.9±0.2° 2θ as measured using Cu Kα.radiation;
(b) a Differential Scanning calorimetry (DSC) thermogram with an exothermic peak having a peak temperature at about 187.3 [symbol] ;
(c) a Thermogravimetric Thermal Analysis (TGA) thermogram exhibiting a mass loss of about 3.28% from the onset of heating up to approximately 150.0 [symbol] ;
(d) a Thermogravimetric Thermal Analysis (TGA) thermogram exhibiting a mass loss of about 27.31% from the onset of heating from 150 [symbol] to 225 [symbol] ; or
(e) combinations thereof.

In some embodiments of Compound 1 Succinate salt Type A, the crystalline form has an X-ray powder diffraction (XRPD) pattern substantially the same as shown in FIG. 26 as measured using Cu Kα.radiation.

In some embodiments of Compound 1 Succinate salt Type A, the crystalline form has an X-ray powder diffraction (XRPD) pattern with peaks found in Table 12 as measured using Cu Kα.radiation.

In some embodiments of Compound 1 Succinate salt Type A, the crystalline form has an X-ray powder diffraction (XRPD) pattern with peaks at 9.4±0.2° 2θ, 15.1±0.2° 2θ, and 19.9±0.2° 2θ as measured using Cu Kα.radiation.

In some embodiments of Compound 1 Succinate salt Type A, the X-ray powder diffraction (XRPD) pattern further comprises peaks at 13.9±0.2° 2θ, 16.6±0.2° 2θ, and 23.0±0.2° 2θ as measured using Cu Kα.radiation.

In some embodiments of Compound 1 Succinate salt Type A, the X-ray powder diffraction (XRPD) pattern further comprises peaks at 8.3±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with at least two peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with at least three peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with at least four peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with at least five peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with at least six peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the crystalline form has an X-ray powder diffraction (XRPD) pattern with at least seven peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 20, and 27.2±0.2° 2θ as measured using Cu Kα.radiation.

In some embodiments of Compound 1 Succinate salt Type A, the crystalline form has an X-ray powder diffraction (XRPD) pattern with at least eight peaks at 8.3±0.2° 2θ, 9.4±0.2° 2θ, 13.9±0.2° 2θ, 15.1±0.2° 2θ, 16.6±0.2° 2θ, 19.9±0.2° 2θ, 23.0±0.2° 2θ, 24.4±0.2° 2θ, and 27.2±0.2° 2θ as measured using Cu Kα.radiation.

In some embodiments of Compound 1 Succinate salt Type A, the Differential Scanning calorimetry (DSC) thermogram is substantially the same as shown in FIG. 27.

In some embodiments of Compound 1 Succinate salt Type A, the Differential Scanning calorimetry (DSC) thermogram has an exothermic peak having a peak temperature at about 187.3 [symbol] .

In some embodiments of Compound 1 Succinate salt Type A, the Thermogravimetric Thermal Analysis (TGA) thermogram is substantially the same as shown in FIG. 27.

In some embodiments of Compound 1 Succinate salt Type A, the Thermogravimetric Thermal Analysis (TGA) thermogram exhibits a mass loss of about 3.28% from the onset of heating up to approximately 150.0 [symbol] .

In some embodiments of Compound 1 Succinate salt Type A, the Thermogravimetric Thermal Analysis (TGA) thermogram exhibits a mass loss of about 27.31% from the onset of heating from 150 [symbol] to 225 [symbol] .

In some embodiments of Compound 1 Succinate salt Type A, the molar ratio of succinic acid to Compound 1 freebase is about 0.8:1.

TABLE 12
X-Ray Powder Diffraction peaks of succinate Type A

| Pos. [°2θ] | Rel. Int. [%] |
|---|---|
| 6.86 | 6.85 |
| 8.29 | 13.21 |
| 9.41 | 87.90 |
| 11.72 | 12.31 |
| 13.31 | 10.67 |
| 13.91 | 17.82 |
| 15.12 | 100.00 |
| 16.61 | 18.34 |
| 17.58 | 7.13 |
| 18.81 | 7.88 |
| 19.10 | 14.78 |
| 19.63 | 47.86 |
| 19.89 | 55.97 |
| 20.66 | 5.21 |
| 22.58 | 5.20 |
| 23.02 | 39.91 |
| 23.27 | 22.58 |
| 23.80 | 6.67 |
| 24.16 | 7.32 |
| 24.44 | 20.29 |
| 25.03 | 14.76 |
| 25.65 | 14.64 |
| 26.25 | 3.35 |
| 26.78 | 9.34 |
| 27.20 | 30.12 |
| 27.41 | 16.88 |
| 28.16 | 14.57 |
| 29.05 | 4.55 |
| 29.40 | 2.80 |
| 31.13 | 3.42 |
| 32.77 | 2.28 |
| 33.86 | 1.05 |
| 35.19 | 2.18 |
| 36.57 | 4.29 |

### Compound 1 Tosylate Salt Type A

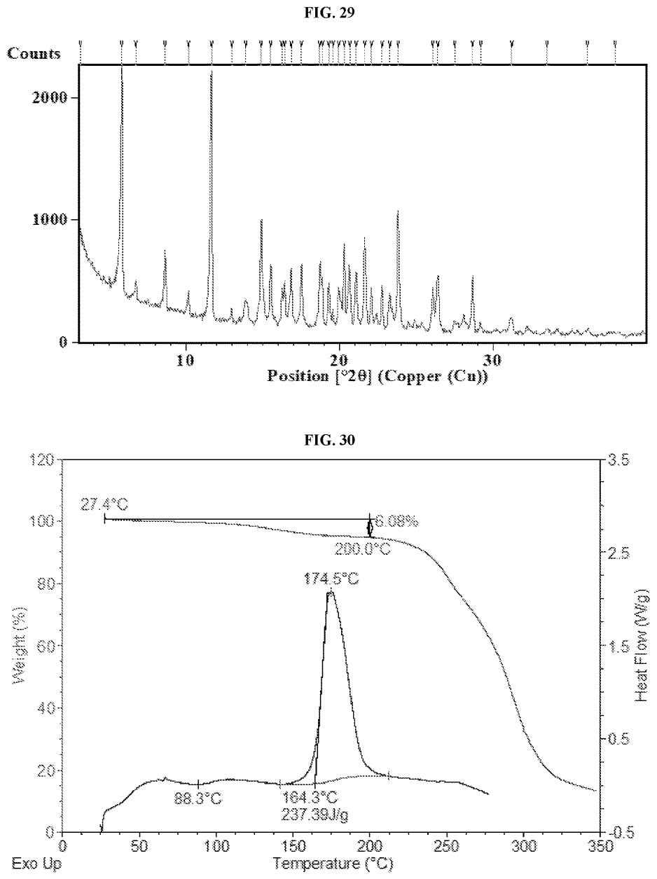

Disclosed herein is Compound 1 Tosylate salt Type A. In some embodiments, the crystalline form is Compound 1 Tosylate salt Type A characterized as having at least one of the following properties:
(a) an X-Ray powder diffraction (XRPD) pattern substantially the same as shown in FIG. 29 as measured using Cu Kα.radiation;
(b) an X-Ray powder diffraction (XRPD) pattern with peaks at 5.8±0.2° 2θ, 11.7±0.2° 2θ, and 14.9±0.2° 2θ as measured using Cu Kα.radiation;
(c) a Differential Scanning calorimetry (DSC) thermogram substantially the same as shown in FIG. 30;
(d) a Differential Scanning calorimetry (DSC) thermogram with an exothermic peak having a peak temperature at about 174.5 [symbol] ;
(e) a Differential Scanning calorimetry (DSC) thermogram with an endothermic peak having a peak temperature at about 88.3 [symbol] ;
(f) a Thermogravimetric Thermal Analysis (TGA) thermogram substantially the same as shown in FIG. 30;
(g) a Thermogravimetric Thermal Analysis (TGA) thermogram exhibiting a mass loss of about 6.08% from the onset of heating up to approximately 200.0; or
(h) combinations thereof.

In some embodiments, the crystalline form is Compound 1 Tosylate salt Type A characterized as having at least one of the following properties:
(a) an X-Ray powder diffraction (XRPD) pattern substantially the same as shown in FIG. 29 as measured using Cu Kα.radiation;
(b) an X-Ray powder diffraction (XRPD) pattern with peaks at 5.8±0.2° 2θ, 11.7±0.2° 20, and 14.9±0.2° 2θ as measured using Cu Kα.radiation;
(c) a Differential Scanning calorimetry (DSC) thermogram substantially the same as shown in FIG. 30;
(d) a Thermogravimetric Thermal Analysis (TGA) thermogram substantially the same as shown in FIG. 30; or
(e) combinations thereof.

In some embodiments, the crystalline form is Compound 1 Tosylate salt Type A characterized as having at least one of the following properties:
(a) an X-Ray powder diffraction (XRPD) pattern with peaks at 5.8±0.2° 2θ, 11.7±0.2° 2θ, and 14.9±0.2° 2θ as measured using Cu Kα.radiation;
(b) a Differential Scanning calorimetry (DSC) thermogram with an exothermic peak having a peak temperature at about 174.5 [symbol] ;
(c) a Differential Scanning calorimetry (DSC) thermogram with an endothermic peak having a peak temperature at about 88.3 [symbol] ;
(d) a Thermogravimetric Thermal Analysis (TGA) thermogram exhibiting a mass loss of about 6.08% from the onset of heating up to approximately 200.0; or
(e) combinations thereof.

In some embodiments of Compound 1 Tosylate salt Type A, the crystalline form has an X-ray powder diffraction (XRPD) pattern substantially the same as shown in FIG. 29 as measured using Cu Kα.radiation.

In some embodiments of Compound 1 Tosylate salt Type A, the crystalline form has an X-ray powder diffraction (XRPD) pattern with peaks found in Table 13 as measured using Cu Kα.radiation.

In some embodiments of Compound 1 Tosylate salt Type A, the crystalline form has an X-ray powder diffraction (XRPD) pattern with peaks at 5.8±0.2° 2θ, 11.7±0.2° 2θ, and 14.9±0.2° 2θ as measured using Cu Kα.radiation.

In some embodiments, the X-ray powder diffraction (XRPD) pattern further comprises peaks at 8.7±0.2° 2θ, 15.5±0.2° 2θ, and 17.5±0.2° 2θ as measured using Cu Kα.radiation.

In some embodiments of Compound 1 Tosylate salt Type A, the X-ray powder diffraction (XRPD) pattern further comprises peaks at 18.7±0.2° 2θ, 20.3±0.2° 2θ, and 23.8±0.2° 2θ as measured using Cu Kα.radiation.

In some embodiments of Compound 1 Tosylate salt Type A, the crystalline form has an X-ray powder diffraction (XRPD) pattern with peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 2θ, and 23.8±0.2° 2θ as measured using Cu Kα.radiation.

In some embodiments of Compound 1 Tosylate salt Type A, the crystalline form has an X-ray powder diffraction (XRPD) pattern with at least two peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 20, and 23.8±0.2° 2θ as measured using Cu Kα.radiation.

In some embodiments of Compound 1 Tosylate salt Type A, the crystalline form has an X-ray powder diffraction (XRPD) pattern with at least three peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 2θ, and 23.8±0.2° 2θ as measured using Cu Kα.radiation.

In some embodiments of Compound 1 Tosylate salt Type A, the crystalline form has an X-ray powder diffraction (XRPD) pattern with at least four peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 2θ, and 23.8±0.2° 2θ as measured using Cu Kα.radiation.

In some embodiments of Compound 1 Tosylate salt Type A, the crystalline form has an X-ray powder diffraction (XRPD) pattern with at least five peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 2θ, and 23.8±0.2° 2θ as measured using Cu Kα.radiation.

In some embodiments of Compound 1 Tosylate salt Type A, the crystalline form has an X-ray powder diffraction (XRPD) pattern with at least six peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 20, and 23.8±0.2° 2θ as measured using Cu Kα.radiation.

In some embodiments of Compound 1 Tosylate salt Type A, the crystalline form has an X-ray powder diffraction (XRPD) pattern with at least seven peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 2θ, and 23.8±0.2° 2θ as measured using Cu Kα.radiation.

In some embodiments of Compound 1 Tosylate salt Type A, the crystalline form has an X-ray powder diffraction (XRPD) pattern with at least eight peaks at 5.8±0.2° 2θ, 8.7±0.2° 2θ, 11.7±0.2° 2θ, 14.9±0.2° 2θ, 15.5±0.2° 2θ, 17.5±0.2° 2θ, 18.7±0.2° 2θ, 20.3±0.2° 2θ, and 23.8±0.2° 2θ as measured using Cu Kα.radiation.

In some embodiments of Compound 1 Tosylate salt Type A, the Differential Scanning calorimetry (DSC) thermogram is substantially the same as shown in FIG. 30.

In some embodiments of Compound 1 Tosylate salt Type A, the Differential Scanning calorimetry (DSC) thermogram has an exothermic peak having a peak temperature at about 174.5 [symbol] .

In some embodiments of Compound 1 Tosylate salt Type A, the Differential Scanning calorimetry (DSC) thermogram has an endothermic peak having a peak temperature at about 88.3 [symbol] .

In some embodiments of Compound 1 Tosylate salt Type A, the Thermogravimetric Thermal Analysis (TGA) thermogram is substantially the same as shown in FIG. 30.

In some embodiments of Compound 1 Tosylate salt Type A, the Thermogravimetric Thermal Analysis (TGA) thermogram exhibits a mass loss of about 6.08% from the onset of heating up to approximately 200.0.

In some embodiments of Compound 1 Tosylate salt Type A, the molar ratio of p-toluenesulfonic acid to Compound 1 freebase is about 1:1.

TABLE 13
X-Ray Powder Diffraction peaks of Tosylate Type A

| Pos. [°2θ] | Rel. Int. [%] |
|---|---|
| 3.15 | 5.55 |
| 5.84 | 90.97 |
| 6.72 | 6.45 |
| 8.66 | 23.79 |
| 10.18 | 10.35 |
| 11.67 | 100.00 |
| 12.98 | 5.67 |
| 13.91 | 8.97 |
| 14.91 | 42.61 |
| 15.52 | 24.16 |
| 16.26 | 12.40 |
| 16.44 | 17.52 |
| 16.86 | 22.48 |
| 17.54 | 25.03 |
| 18.73 | 25.33 |
| 18.89 | 18.13 |
| 19.32 | 17.68 |
| 19.58 | 7.00 |
| 19.95 | 15.81 |
| 20.30 | 33.62 |
| 20.67 | 25.20 |
| 21.08 | 22.22 |
| 21.65 | 33.56 |
| 22.08 | 15.93 |
| 22.77 | 17.29 |
| 23.27 | 13.47 |
| 23.81 | 47.72 |
| 26.07 | 17.38 |
| 26.41 | 21.99 |
| 27.49 | 4.24 |
| 28.67 | 22.54 |
| 29.18 | 4.19 |
| 31.21 | 6.64 |
| 33.51 | 2.46 |
| 36.13 | 2.25 |
| 37.93 | 1.17 |

### Compound 1 Tosylate Salt Type B

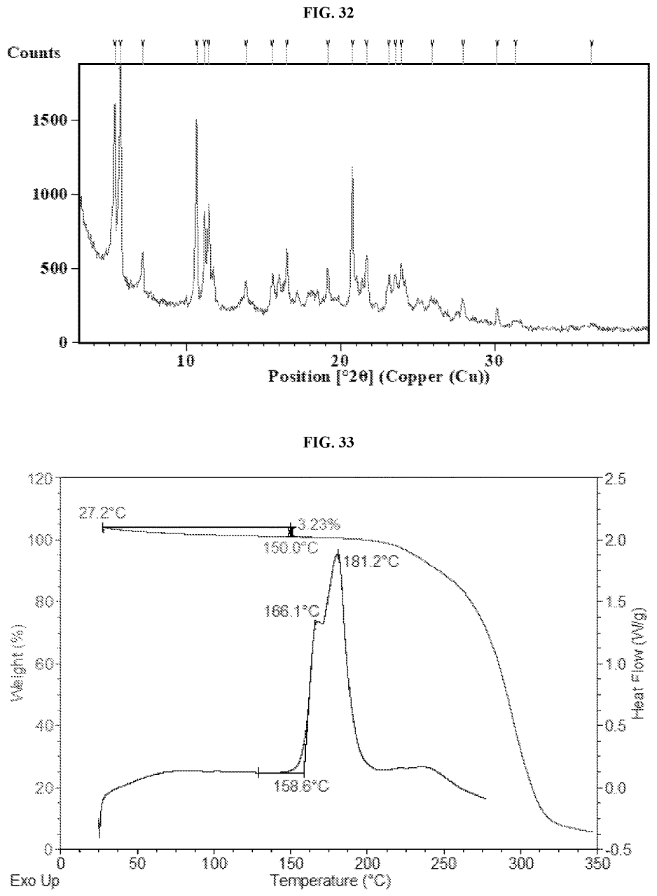

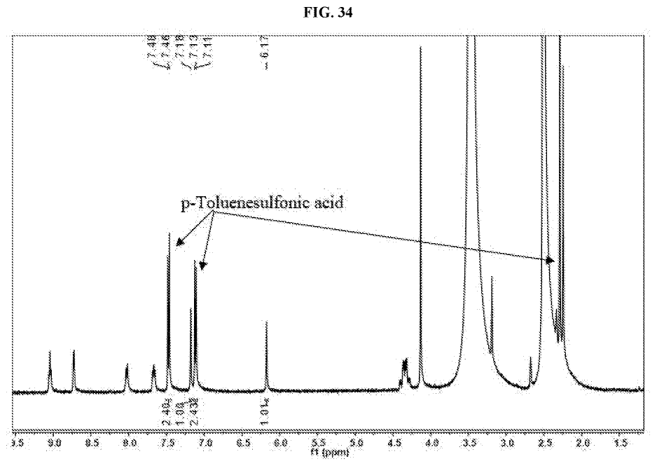

Disclosed herein is Compound 1 Tosylate salt Type B. In some embodiments, the crystalline form is Compound 1 Tosylate salt Type B characterized as having at least one of the following properties:
(a) an X-Ray powder diffraction (XRPD) pattern substantially the same as shown in FIG. 32 as measured using Cu Kα.radiation;
(b) an X-Ray powder diffraction (XRPD) pattern with peaks at 5.8±0.2° 2θ, 10.7±0.2° 2θ, and 16.5±0.2° 2θ as measured using Cu Kα.radiation;
(c) a Differential Scanning calorimetry (DSC) thermogram substantially the same as shown in FIG. 33;
(d) a Differential Scanning calorimetry (DSC) thermogram with an exothermic peak having a peak temperature at about 166.1 [symbol] ;
(e) a Differential Scanning calorimetry (DSC) thermogram with an exothermic peak having a peak temperature at about 181.2 [symbol] ;
(f) a Thermogravimetric Thermal Analysis (TGA) thermogram substantially the same as shown in FIG. 33;
(g) a Thermogravimetric Thermal Analysis (TGA) thermogram exhibiting a mass loss of about 3.23% from the onset of heating up to approximately 150.0 [symbol] ; or
(h) combinations thereof.

In some embodiments, the crystalline form is Compound 1 Tosylate salt Type B characterized as having at least one of the following properties:
(a) an X-Ray powder diffraction (XRPD) pattern substantially the same as shown in FIG. 32 as measured using Cu Kα.radiation;
(b) an X-Ray powder diffraction (XRPD) pattern with peaks at 5.8±0.2° 2θ, 10.7±0.2° 2θ, and 16.5±0.2° 2θ as measured using Cu Kα.radiation;
(c) a Differential Scanning calorimetry (DSC) thermogram substantially the same as shown in FIG. 33;
(d) a Thermogravimetric Thermal Analysis (TGA) thermogram substantially the same as shown in FIG. 33; or
(e) combinations thereof.

In some embodiments, the crystalline form is Compound 1 Tosylate salt Type B characterized as having at least one of the following properties:
(a) an X-Ray powder diffraction (XRPD) pattern with peaks at 5.8±0.2° 2θ, 10.7±0.2° 2θ, and 16.5±0.2° 2θ as measured using Cu Kα.radiation;
(b) a Differential Scanning calorimetry (DSC) thermogram with an exothermic peak having a peak temperature at about 166.1 [symbol] ;
(c) a Differential Scanning calorimetry (DSC) thermogram with an exothermic peak having a peak temperature at about 181.2 [symbol] ;
(d) a Thermogravimetric Thermal Analysis (TGA) thermogram exhibiting a mass loss of about 3.23% from the onset of heating up to approximately 150.0 [symbol] ; or
(e) combinations thereof.

In some embodiments of Compound 1 Tosylate salt Type B, the crystalline form has an X-ray powder diffraction (XRPD) pattern substantially the same as shown in FIG. 32 as measured using Cu Kα.radiation.

In some embodiments of Compound 1 Tosylate salt Type B, the crystalline form has an X-ray powder diffraction (XRPD) pattern with peaks found in Table 14 as measured using Cu Kα.radiation.

In some embodiments of Compound 1 Tosylate salt Type B, the crystalline form has an X-ray powder diffraction (XRPD) pattern with peaks at 5.8±0.2° 2θ, 10.7±0.2° 2θ, and 16.5±0.2° 2θ as measured using Cu Kα.radiation.

In some embodiments of Compound 1 Tosylate salt Type B, the X-ray powder diffraction (XRPD) pattern further comprises peaks at 11.2±0.2° 2θ, 11.5±0.2° 2θ, and 20.8±0.2° 2θ as measured using Cu Kα.radiation.

In some embodiments of Compound 1 Tosylate salt Type B, the crystalline form has an X-ray powder diffraction (XRPD) pattern with peaks at 5.8±0.2° 2θ, 10.7±0.2° 2θ, 11.2±0.2° 2θ, 11.5±0.2° 2θ, 16.5±0.2° 2θ, and 20.8±0.2° 2θ as measured using Cu Kα.radiation.

In some embodiments of Compound 1 Tosylate salt Type B, the crystalline form has an X-ray powder diffraction (XRPD) pattern with at least two peaks at 5.8±0.2° 2θ, 10.7±0.2° 2θ, 11.2±0.2° 2θ, 11.5±0.2° 2θ, 16.5±0.2° 2θ, and 20.8±0.2° 2θ as measured using Cu Kα.radiation.

In some embodiments of Compound 1 Tosylate salt Type B, the crystalline form has an X-ray powder diffraction (XRPD) pattern with at least three peaks at 5.8±0.2° 2θ, 10.7±0.2° 2θ, 11.2±0. [symbol] 2θ, 11.5±0.2° 2θ, 16.5±0.2° 2θ, and 20.8±0.2° 2θ as measured using Cu Kα.radiation.

In some embodiments of Compound 1 Tosylate salt Type B, the crystalline form has an X-ray powder diffraction (XRPD) pattern with at least four peaks at 5.8=±0.2° 2θ, 10.7±0.2° 2θ, 11.2±0.2° 2θ, 11.5±0.2° 2θ, 16.5±0.2° 2θ, and 20.8±0.2° 2θ as measured using Cu Kα.radiation.

In some embodiments of Compound 1 Tosylate salt Type B, the crystalline form has an X-ray powder diffraction (XRPD) pattern with at least five peaks at 5.8±0.2° 2θ, 10.7±0.2° 2θ, 11.2±0.2° 2θ, 11.5±0.2° 2θ, 16.5±0.2° 2θ, and 20.8±0.2° 2θ as measured using Cu Kα.radiation.

In some embodiments of Compound 1 Tosylate salt Type B, the Differential Scanning calorimetry (DSC) thermogram is substantially the same as shown in FIG. 33.

In some embodiments of Compound 1 Tosylate salt Type B, the Differential Scanning calorimetry (DSC) thermogram has an exothermic peak having a peak temperature at about 166.1 [symbol] .

In some embodiments of Compound 1 Tosylate salt Type B, the Differential Scanning calorimetry (DSC) thermogram has an exothermic peak having a peak temperature at about 181.2 [symbol] .

In some embodiments of Compound 1 Tosylate salt Type B, the Thermogravimetric Thermal Analysis (TGA) thermogram is substantially the same as shown in FIG. 33.

In some embodiments of Compound 1 Tosylate salt Type B, the Thermogravimetric Thermal Analysis (TGA) thermogram exhibits a mass loss of about 3.23% from the onset of heating up to approximately 150.0 [symbol] .

In some embodiments of Compound 1 Tosylate salt Type B, the molar ratio of p-toluenesulfonic acid to Compound 1 freebase is about 1.2:1.

TABLE 14
X-Ray Powder Diffraction peaks of Tosylate Type B

| Pos. [°2θ] | Rel. Int. [%] |
|---|---|
| 5.36 | 79.27 |
| 5.75 | 100.00 |
| 7.17 | 20.07 |
| 10.67 | 88.53 |
| 11.18 | 46.14 |
| 11.47 | 47.77 |
| 13.86 | 13.70 |
| 15.56 | 14.91 |
| 16.53 | 29.20 |
| 19.18 | 19.17 |
| 20.77 | 67.67 |
| 21.70 | 26.17 |
| 23.13 | 15.52 |
| 23.56 | 18.44 |
| 23.94 | 22.82 |
| 25.93 | 8.07 |
| 27.93 | 9.80 |
| 30.17 | 7.64 |
| 31.34 | 2.95 |
| 36.27 | 2.61 |

### Compound 1 Tosylate Salt Type C

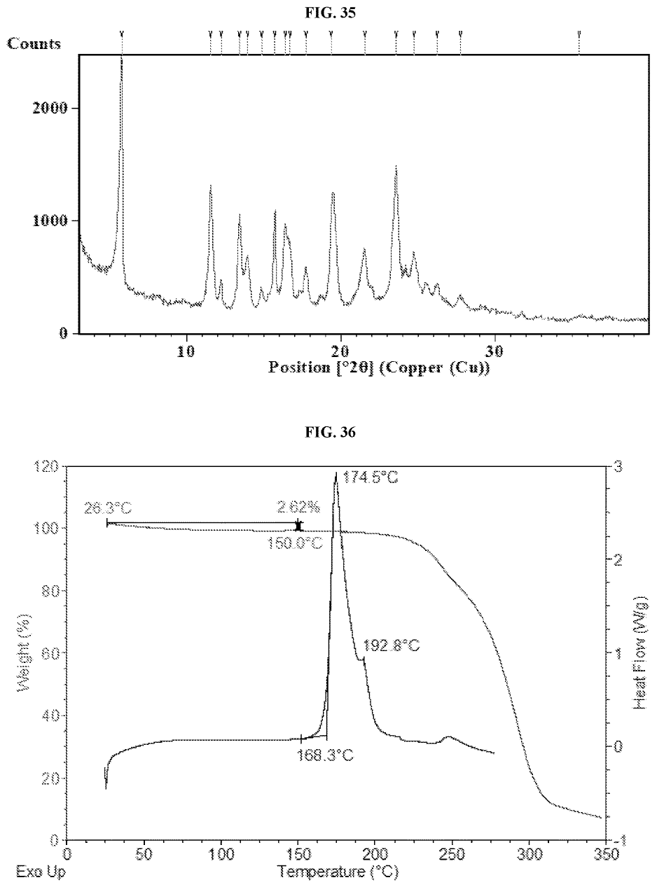

Disclosed herein is Compound 1 Tosylate salt Type C. In some embodiments, the crystalline form is Compound 1 Tosylate salt Type C characterized as having at least one of the following properties:
(a) an X-Ray powder diffraction (XRPD) pattern substantially the same as shown in FIG. 35 as measured using Cu Kα.radiation;
(b) an X-Ray powder diffraction (XRPD) pattern with peaks at 5.8±0. [symbol] 2θ, 11.6±0.2° 2θ, and 15.7±0.2° 2θ as measured using Cu Kα.radiation;
(c) a Differential Scanning calorimetry (DSC) thermogram substantially the same as shown in FIG. 36;
(d) a Differential Scanning calorimetry (DSC) thermogram with an exothermic peak having a peak temperature at about 174.5 [symbol] ;
(e) a Differential Scanning calorimetry (DSC) thermogram with an exothermic peak having a peak temperature at about 192.8 [symbol] ;
(f) a Thermogravimetric Thermal Analysis (TGA) thermogram substantially the same as shown in FIG. 36;
(g) a Thermogravimetric Thermal Analysis (TGA) thermogram exhibiting a mass loss of about 2.62% from the onset of heating up to approximately 150.0 [symbol] ; or
(h) combinations thereof.

In some embodiments, the crystalline form is Compound 1 Tosylate salt Type C characterized as having at least one of the following properties:
(a) an X-Ray powder diffraction (XRPD) pattern substantially the same as shown in FIG. 35 as measured using Cu Kα.radiation;
(b) an X-Ray powder diffraction (XRPD) pattern with peaks at 5.8±0.2° 2θ, 11.6±0.2° 2θ, and 15.7±0.2° 2θ as measured using Cu Kα.radiation;
(c) a Differential Scanning calorimetry (DSC) thermogram substantially the same as shown in FIG. 36;
(d) a Thermogravimetric Thermal Analysis (TGA) thermogram substantially the same as shown in FIG. 36; or
(e) combinations thereof.

In some embodiments, the crystalline form is Compound 1 Tosylate salt Type C characterized as having at least one of the following properties:
(a) an X-Ray powder diffraction (XRPD) pattern with peaks at 5.8±0.2° 2θ, 11.6±0.2° 2θ, and 15.7±0.2° 2θ as measured using Cu Kα.radiation;
(b) a Differential Scanning calorimetry (DSC) thermogram with an exothermic peak having a peak temperature at about 174.5 [symbol] ;
(c) a Differential Scanning calorimetry (DSC) thermogram with an exothermic peak having a peak temperature at about 192.8 [symbol] ;
(d) a Thermogravimetric Thermal Analysis (TGA) thermogram exhibiting a mass loss of about 2.62% from the onset of heating up to approximately 150.0 [symbol] ; or
(e) combinations thereof.

In some embodiments of Compound 1 Tosylate salt Type C, the crystalline form has an X-ray powder diffraction (XRPD) pattern substantially the same as shown in FIG. 35 as measured using Cu Kα.radiation.

In some embodiments of Compound 1 Tosylate salt Type C, the crystalline form has an X-ray powder diffraction (XRPD) pattern with peaks found in Table 15 as measured using Cu Kα.radiation.

In some embodiments of Compound 1 Tosylate salt Type C, the crystalline form has an X-ray powder diffraction (XRPD) pattern with peaks at 5.8±0.2° 2θ, 11.6±0.2° 2θ, and 15.7±0.2° 2θ as measured using Cu Kα.radiation.

In some embodiments of Compound 1 Tosylate salt Type C, the X-ray powder diffraction (XRPD) pattern further comprises peaks at 13.4±0.2° 2θ, 14.0±0.2° 2θ, and 16.4±0.2° 2θ as measured using Cu Kα.radiation.

In some embodiments of Compound 1 Tosylate salt Type C, the X-ray powder diffraction (XRPD) pattern further comprises peaks at 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least two peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least three peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least four peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least five peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least six peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least seven peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 2θ, and 23.6±0.2° 2θ as measured using Cu Kα.radiation.

In some embodiments of Compound 1 Tosylate salt Type C, the crystalline form has an X-ray powder diffraction (XRPD) pattern with at least eight peaks at 5.8±0.2° 2θ, 11.6±0.2° 2θ, 13.4±0.2° 2θ, 14.0±0.2° 2θ, 15.7±0.2° 2θ, 16.4±0.2° 2θ, 17.8±0.2° 2θ, 19.4±0.2° 20, and 23.6±0.2° 2θ as measured using Cu Kα.radiation.

In some embodiments of Compound 1 Tosylate salt Type C, the Differential Scanning calorimetry (DSC) thermogram is substantially the same as shown in FIG. 36.

In some embodiments of Compound 1 Tosylate salt Type C, the Differential Scanning calorimetry (DSC) thermogram has an exothermic peak having a peak temperature at about 174.5 [symbol] .

In some embodiments of Compound 1 Tosylate salt Type C, the Differential Scanning calorimetry (DSC) thermogram has an exothermic peak having a peak temperature at about 192.8 [symbol] .

In some embodiments of Compound 1 Tosylate salt Type C, the Thermogravimetric Thermal Analysis (TGA) thermogram is substantially the same as shown in FIG. 36.

In some embodiments of Compound 1 Tosylate salt Type C, the Thermogravimetric Thermal Analysis (TGA) thermogram exhibits a mass loss of about 2.62% from the onset of heating up to approximately 150.0 [symbol] .

In some embodiments of Compound 1 Tosylate salt Type C, the molar ratio of p-toluenesulfonic acid to Compound 1 freebase is about 1:1.

TABLE 15
X-Ray Powder Diffraction peaks of Tosylate Type C

| Pos. [°2θ] | Rel. Int. [%] |
|---|---|
| 5.80 | 100.00 |
| 11.57 | 57.24 |
| 12.25 | 13.38 |
| 13.42 | 39.99 |
| 13.96 | 23.12 |
| 14.85 | 8.38 |
| 15.72 | 44.91 |
| 16.39 | 37.59 |
| 16.72 | 27.47 |
| 17.75 | 17.41 |
| 19.38 | 42.60 |
| 21.56 | 25.19 |
| 23.59 | 63.94 |
| 24.75 | 24.09 |
| 26.26 | 10.67 |
| 27.76 | 5.79 |
| 35.45 | 2.10 |

### Compound 1 Mesylate Salt Type A

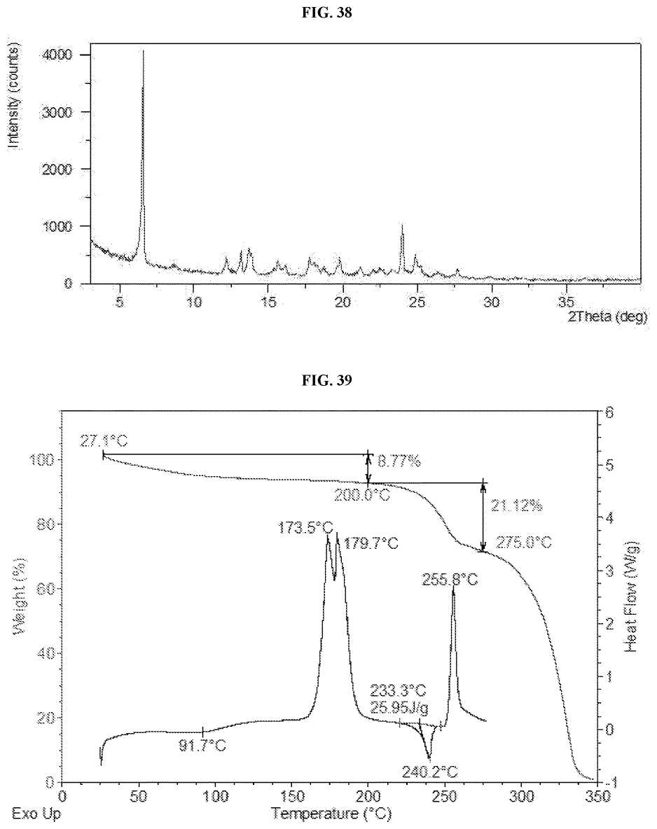

Disclosed herein is Compound 1 Mesylate salt Type A. In some embodiments, the crystalline form is Compound 1 Mesylate salt Type A characterized as having at least one of the following properties:
(a) an X-Ray powder diffraction (XRPD) pattern substantially the same as shown in FIG. 38 as measured using Cu Kα.radiation;
(b) an X-Ray powder diffraction (XRPD) pattern with peaks at 6.6±0.2° 2θ, 13.2±0.2° 2θ, and 13.7±0.2° 2θ as measured using Cu Kα.radiation;
(c) a Differential Scanning calorimetry (DSC) thermogram substantially the same as shown in FIG. 39;
(d) a Differential Scanning calorimetry (DSC) thermogram with an exothermic peak having a peak temperature at about 173.5 [symbol] ;
(e) a Differential Scanning calorimetry (DSC) thermogram with an exothermic peak having a peak temperature at about 179.7 [symbol] ;
(f) a Differential Scanning calorimetry (DSC) thermogram with an exothermic peak having a peak temperature at about 255.8 [symbol] ;
(g) a Differential Scanning calorimetry (DSC) thermogram with an endothermic peak having a peak temperature at about 91.7 [symbol] ;
(h) a Differential Scanning calorimetry (DSC) thermogram with an endothermic peak having a peak temperature at about 240.2 [symbol] ;
(i) a Thermogravimetric Thermal Analysis (TGA) thermogram substantially the same as shown in FIG. 39;
(j) a Thermogravimetric Thermal Analysis (TGA) thermogram exhibiting a mass loss of about 8.77% from the onset of heating up to approximately 200.0 [symbol] ;
(k) a Thermogravimetric Thermal Analysis (TGA) thermogram exhibiting a mass loss of about 21.12% from the onset of heating from 200 [symbol] to 275 [symbol] ; or
(l) combinations thereof.

In some embodiments, the crystalline form is Compound 1 Mesylate salt Type A characterized as having at least one of the following properties:
(a) an X-Ray powder diffraction (XRPD) pattern substantially the same as shown in FIG. 38 as measured using Cu Kα.radiation;
(b) an X-Ray powder diffraction (XRPD) pattern with peaks at 6.6±0.2° 2θ, 13.2±0.2° 2θ, and 13.7±0.2° 2θ as measured using Cu Kα.radiation;
(c) a Differential Scanning calorimetry (DSC) thermogram substantially the same as shown in FIG. 39;
(d) a Thermogravimetric Thermal Analysis (TGA) thermogram substantially the same as shown in FIG. 39; or
(e) combinations thereof.

In some embodiments, the crystalline form is Compound 1 Mesylate salt Type A characterized as having at least one of the following properties:
(a) an X-Ray powder diffraction (XRPD) pattern with peaks at 6.6±0.2° 2θ, 13.2±0.2° 2θ, and 13.7±0.2° 2θ as measured using Cu Kα.radiation;
(b) a Differential Scanning calorimetry (DSC) thermogram with an exothermic peak having a peak temperature at about 173.5 [symbol] ;
(c) a Differential Scanning calorimetry (DSC) thermogram with an exothermic peak having a peak temperature at about 179.7 [symbol] ;
(d) a Differential Scanning calorimetry (DSC) thermogram with an exothermic peak having a peak temperature at about 255.8 [symbol] ;
(e) a Differential Scanning calorimetry (DSC) thermogram with an endothermic peak having a peak temperature at about 91.7 [symbol] ;
(f) a Differential Scanning calorimetry (DSC) thermogram with an endothermic peak having a peak temperature at about 240.2 [symbol] ;
(g) a Thermogravimetric Thermal Analysis (TGA) thermogram exhibiting a mass loss of about 8.77% from the onset of heating up to approximately 200.0 [symbol] ;
(h) a Thermogravimetric Thermal Analysis (TGA) thermogram exhibiting a mass loss of about 21.12% from the onset of heating from 200 [symbol] to 275 [symbol] ; or
(i) combinations thereof.

In some embodiments of Compound 1 Mesylate salt Type A, the crystalline form has an X-ray powder diffraction (XRPD) pattern substantially the same as shown in FIG. 38 as measured using Cu Kα.radiation.

In some embodiments of Compound 1 Mesylate salt Type A, the crystalline form has an X-ray powder diffraction (XRPD) pattern with peaks found in Table 16 as measured using Cu Kα.radiation.

In some embodiments of Compound 1 Mesylate salt Type A, the crystalline form has an X-ray powder diffraction (XRPD) pattern with peaks at 6.6±0.2° 2θ, 13.2±0.2° 2θ, and 13.7±0.2° 2θ as measured using Cu Kα.radiation.

In some embodiments of Compound 1 Mesylate salt Type A, the X-ray powder diffraction (XRPD) pattern further comprises peaks at 12.2±0.2° 2θ, 15.6±0.2° 2θ, and 24.0±0.2° 2θ as measured using Cu Kα.radiation.

In some embodiments of Compound 1 Mesylate salt Type A, the crystalline form has an X-ray powder diffraction (XRPD) pattern with peaks at 6.6±0.2° 2θ, 12.2±0.2° 2θ, 13.2±0.2° 2θ, 13.7±0.2° 2θ, 12.2±0.2° 2θ, 15.6±0.2° 2θ, and 24.0±0.2° 2θ as measured using Cu Kα.radiation.

In some embodiments of Compound 1 Mesylate salt Type A, the crystalline form has an X-ray powder diffraction (XRPD) pattern with at least two peaks at 6.6±0.2° 2θ, 12.2±0.2° 2θ, 13.2±0.2° 2θ, 13.7±0.2° 2θ, 12.2±0.2° 2θ, 15.6±0.2° 2θ, and 24.0±0.2° 2θ as measured using Cu Kα.radiation.

In some embodiments of Compound 1 Mesylate salt Type A, the crystalline form has an X-ray powder diffraction (XRPD) pattern with at least three peaks at 6.6±0.2° 2θ, 12.2±0.2° 2θ, 13.2±0.2° 2θ, 13.7±0.2° 2θ, 12.2±0.2° 2θ, 15.6±0.2° 2θ, and 24.0±0.2° 2θ as measured using Cu Kα.radiation.

In some embodiments of Compound 1 Mesylate salt Type A, the crystalline form has an X-ray powder diffraction (XRPD) pattern with at least four peaks at 6.6±0.2° 2θ, 12.2±0.2° 2θ, 13.2±0.2° 2θ, 13.7±0.2° 2θ, 12.2±0.2° 2θ, 15.6±0.2° 2θ, and 24.0±0.2° 2θ as measured using Cu Kα.radiation.

In some embodiments of Compound 1 Mesylate salt Type A, the crystalline form has an X-ray powder diffraction (XRPD) pattern with at least five peaks at 6.6±0.2° 2θ, 12.2±0.2° 2θ, 13.2±0.2° 2θ, 13.7±0.2° 2θ, 12.2±0.2° 2θ, 15.6±0.2° 2θ, and 24.0±0.2° 2θ as measured using Cu Kα.radiation.

In some embodiments of Compound 1 Mesylate salt Type A, the Differential Scanning calorimetry (DSC) thermogram is substantially the same as shown in FIG. 39.

In some embodiments of Compound 1 Mesylate salt Type A, the Differential Scanning calorimetry (DSC) thermogram has an exothermic peak having a peak temperature at about 173.5 [symbol] .

In some embodiments of Compound 1 Mesylate salt Type A, the Differential Scanning calorimetry (DSC) thermogram has an exothermic peak having a peak temperature at about 179.7 [symbol] .

In some embodiments of Compound 1 Mesylate salt Type A, the Differential Scanning calorimetry (DSC) thermogram has an exothermic peak having a peak temperature at about 255.8 [symbol] .

In some embodiments of Compound 1 Mesylate salt Type A, the Differential Scanning calorimetry (DSC) thermogram has an endothermic peak having a peak temperature at about 91.7 [symbol] .

In some embodiments of Compound 1 Mesylate salt Type A, the Differential Scanning calorimetry (DSC) thermogram has an endothermic peak having a peak temperature at about 240.2 [symbol] .

In some embodiments of Compound 1 Mesylate salt Type A, the Thermogravimetric Thermal Analysis (TGA) thermogram is substantially the same as shown in FIG. 39.

In some embodiments of Compound 1 Mesylate salt Type A, the Thermogravimetric Thermal Analysis (TGA) thermogram exhibits a mass loss of about 8.77% from the onset of heating up to approximately 200.0 [symbol] .

In some embodiments of Compound 1 Mesylate salt Type A, the Thermogravimetric Thermal Analysis (TGA) thermogram exhibits a mass loss of about 21.12% from the onset of heating from 200 [symbol] to 275 [symbol] .

In some embodiments of Compound 1 Mesylate salt Type A, the molar ratio of methanesulfonic acid to Compound 1 freebase is about 0.9:1.

TABLE 16
X-Ray Powder Diffraction peaks of mesylate Type A

| Pos. [°2θ] | Rel. Int. [%] |
|---|---|
| 6.58 | 100.00 |
| 8.66 | 1.49 |
| 12.17 | 7.82 |
| 13.16 | 11.69 |
| 13.69 | 12.96 |
| 15.63 | 7.06 |
| 16.13 | 5.11 |
| 17.79 | 8.43 |
| 18.69 | 4.19 |
| 19.79 | 8.17 |
| 21.16 | 3.94 |
| 22.07 | 3.15 |
| 24.00 | 25.85 |
| 24.88 | 10.91 |
| 26.41 | 2.28 |
| 27.70 | 4.14 |
| 31.88 | 0.83 |
| 36.23 | 0.97 |

### Compound 1 Besylate Salt Type A

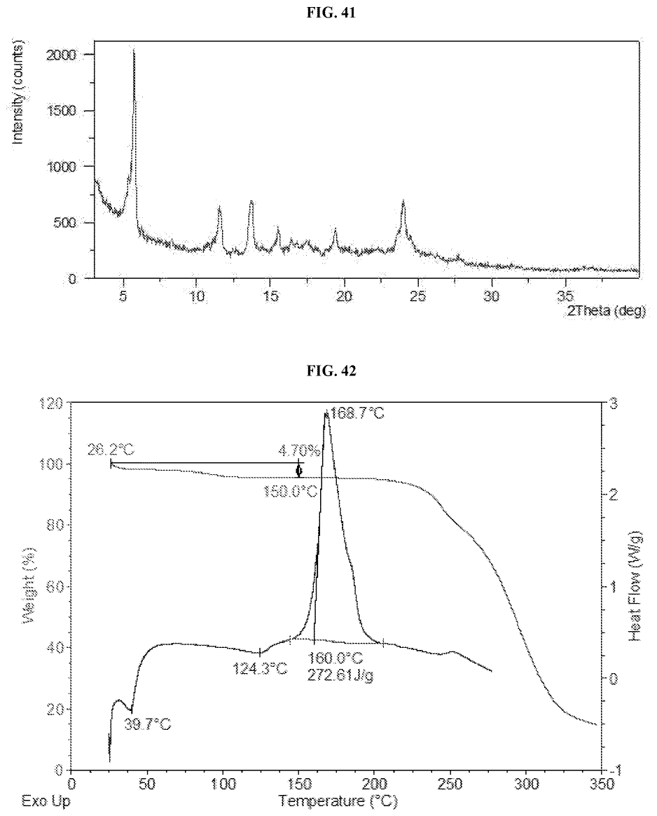

Disclosed herein is Compound 1 Besylate salt Type A. In some embodiments, the crystalline form is Compound 1 Besylate salt Type A characterized as having at least one of the following properties:
(a) an X-Ray powder diffraction (XRPD) pattern substantially the same as shown in FIG. 41 as measured using Cu Kα.radiation;
(b) an X-Ray powder diffraction (XRPD) pattern with peaks at 5.8±0.2° 2θ, 11.5±0.2° 2θ, and 13.8±0.2° 2θ as measured using Cu Kα.radiation;
(c) a Differential Scanning calorimetry (DSC) thermogram substantially the same as shown in FIG. 42;
(d) a Differential Scanning calorimetry (DSC) thermogram with an exothermic peak having a peak temperature at about 168.7 [symbol] ;
(e) a Differential Scanning calorimetry (DSC) thermogram with an endothermic peak having a peak temperature at about 39.7 [symbol] ;
(f) a Differential Scanning calorimetry (DSC) thermogram with an endothermic peak having a peak temperature at about 124.3 [symbol] ;
(g) a Thermogravimetric Thermal Analysis (TGA) thermogram substantially the same as shown in FIG. 42;
(h) a Thermogravimetric Thermal Analysis (TGA) thermogram exhibiting a mass loss of about 4.70% from the onset of heating up to approximately 150.0 [symbol] ; or
(i) combinations thereof.

In some embodiments, the crystalline form is Compound 1 Besylate salt Type A characterized as having at least one of the following properties:
(a) an X-Ray powder diffraction (XRPD) pattern substantially the same as shown in FIG. 41 as measured using Cu Kα.radiation;
(b) an X-Ray powder diffraction (XRPD) pattern with peaks at 5.8±0.2° 2θ, 11.5±0.2° 2θ, and 13.8±0.2° 2θ as measured using Cu Kα.radiation;
(c) a Differential Scanning calorimetry (DSC) thermogram substantially the same as shown in FIG. 42;
(d) a Thermogravimetric Thermal Analysis (TGA) thermogram substantially the same as shown in FIG. 42; or
(e) combinations thereof.

In some embodiments, the crystalline form is Compound 1 Besylate salt Type A characterized as having at least one of the following properties:
(a) an X-Ray powder diffraction (XRPD) pattern with peaks at 5.8±0.2° 2θ, 11.5±0.2° 2θ,
and 13.8±0.2° 2θ as measured using Cu Kα.radiation;
(b) a Differential Scanning calorimetry (DSC) thermogram with an exothermic peak having a peak temperature at about 168.7 [symbol] ;
(c) a Differential Scanning calorimetry (DSC) thermogram with an endothermic peak having a peak temperature at about 39.7 [symbol] ;
(d) a Differential Scanning calorimetry (DSC) thermogram with an endothermic peak having a peak temperature at about 124.3 [symbol] ;
(e) a Thermogravimetric Thermal Analysis (TGA) thermogram exhibiting a mass loss of about 4.70% from the onset of heating up to approximately 150.0 [symbol] ; or
(f) combinations thereof.

In some embodiments of Compound 1 Besylate salt Type A, the crystalline form has an X-ray powder diffraction (XRPD) pattern substantially the same as shown in FIG. 41 as measured using Cu Kα.radiation.

In some embodiments of Compound 1 Besylate salt Type A, the crystalline form has an X-ray powder diffraction (XRPD) pattern with peaks found in Table 17 as measured using Cu Kα.radiation.

In some embodiments of Compound 1 Besylate salt Type A, the crystalline form has an X-ray powder diffraction (XRPD) pattern with peaks at 5.8±0.2° 2θ, 11.5±0.2° 2θ, and 13.8±0.2° 2θ as measured using Cu Kα.radiation.

In some embodiments of Compound 1 Besylate salt Type A, the X-ray powder diffraction (XRPD) pattern further comprises peaks at 15.5±0.2° 2θ, 19.4±0.2° 2θ, and 24.0±0.2° 2θ as measured using Cu Kα.radiation.

In some embodiments of Compound 1 Besylate salt Type A, the crystalline form has an X-ray powder diffraction (XRPD) pattern with peaks at 5.8±0.2° 2θ, 11.5±0.2° 2θ, 13.8±0.2° 2θ, 15.5±0.2° 2θ, 19.4±0.2° 2θ, and 24.0±0.2° 2θ as measured using Cu Kα.radiation.

In some embodiments of Compound 1 Besylate salt Type A, the crystalline form has an X-ray powder diffraction (XRPD) pattern with at least two peaks at 5.8±0.2° 2θ, 11.5±0.2° 2θ, 13.8±0.2° 2θ, 15.5±0.2° 2θ, 19.4±0.2° 2θ, and 24.0±0.2° 2θ as measured using Cu Kα.radiation.

In some embodiments of Compound 1 Besylate salt Type A, the crystalline form has an X-ray powder diffraction (XRPD) pattern with at least three peaks at 5.8±0.2° 2θ, 11.5±0.2° 2θ, 13.8±0. [symbol] 2θ, 15.5±0.2° 2θ, 19.4±0.2° 2θ, and 24.0±0.2° 2θ as measured using Cu Kα.radiation.

In some embodiments of Compound 1 Besylate salt Type A, the crystalline form has an X-ray powder diffraction (XRPD) pattern with at least four peaks at 5.8±0.2° 2θ, 11.5±0.2° 2θ, 13.8±0.2° 2θ, 15.5±0.2° 2θ, 19.4±0.2° 2θ, and 24.0±0.2° 2θ as measured using Cu Kα.radiation.

In some embodiments of Compound 1 Besylate salt Type A, the crystalline form has an X-ray powder diffraction (XRPD) pattern with at least five peaks at 5.8±0.2° 2θ, 11.5±0.2° 2θ, 13.8±0.2° 2θ, 15.5±0.2° 2θ, 19.4±0.2° 2θ, and 24.0±0.2° 2θ as measured using Cu Kα.radiation.

In some embodiments of Compound 1 Besylate salt Type A, the Differential Scanning calorimetry (DSC) thermogram is substantially the same as shown in FIG. 42.

In some embodiments of Compound 1 Besylate salt Type A, the Differential Scanning calorimetry (DSC) thermogram has an exothermic peak having a peak temperature at about 168.7 [symbol] .

In some embodiments of Compound 1 Besylate salt Type A, the Differential Scanning calorimetry (DSC) thermogram has an endothermic peak having a peak temperature at about 39.7 [symbol] .

In some embodiments of Compound 1 Besylate salt Type A, the Differential Scanning calorimetry (DSC) thermogram has an endothermic peak having a peak temperature at about 124.3 [symbol] .

In some embodiments of Compound 1 Besylate salt Type A, the Thermogravimetric Thermal Analysis (TGA) thermogram is substantially the same as shown in FIG. 42.

In some embodiments of Compound 1 Besylate salt Type A, the Thermogravimetric Thermal Analysis (TGA) thermogram exhibits a mass loss of about 4.70% from the onset of heating up to approximately 150.0 [symbol] .

In some embodiments of Compound 1 Besylate salt Type A, the molar ratio of benzenesulfonic acid to Compound 1 freebase is about 1:1.

TABLE 17
X-Ray Powder Diffraction peaks of besylate Type A

| Pos. [°2θ] | Rel. Int. [%] |
|---|---|
| 5.75 | 100.00 |
| 11.48 | 21.98 |
| 13.76 | 28.08 |
| 15.52 | 14.73 |
| 17.49 | 6.54 |
| 19.38 | 13.10 |
| 24.02 | 30.45 |

### Compound 1 Oxalate Salt Type A

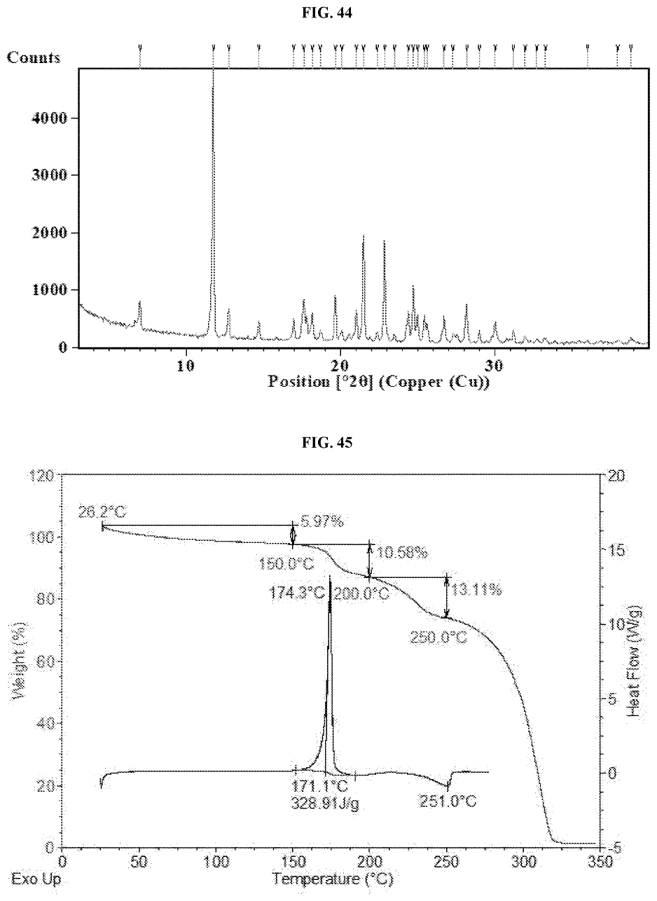

Disclosed herein is Compound 1 Oxalate salt Type A. In some embodiments, the crystalline form is Compound 1 Oxalate salt Type A characterized as having at least one of the following properties:
(a) an X-Ray powder diffraction (XRPD) pattern substantially the same as shown in FIG. 44 as measured using Cu Kα.radiation;
(b) an X-Ray powder diffraction (XRPD) pattern with peaks at 11.8±0.2°2θ, 19.7±0.2° 2θ, and 21.5±0.2° 2θ as measured using Cu Kα.radiation;
(c) a Differential Scanning calorimetry (DSC) thermogram substantially the same as shown in FIG. 45;
(d) a Differential Scanning calorimetry (DSC) thermogram with an exothermic peak having a peak temperature at about 174.3 [symbol] ;
(e) a Differential Scanning calorimetry (DSC) thermogram with an endothermic peak having a peak temperature at about 251.0 [symbol] ;
(f) a Thermogravimetric Thermal Analysis (TGA) thermogram substantially the same as shown in FIG. 45;
(g) a Thermogravimetric Thermal Analysis (TGA) thermogram exhibiting a mass loss of about 5.97% from the onset of heating up to approximately 150.0≙;
(h) a Thermogravimetric Thermal Analysis (TGA) thermogram exhibiting a mass loss of about 10.58% from the onset of heating from 150.0 [symbol] to 200 [symbol] ;
(i) a Thermogravimetric Thermal Analysis (TGA) thermogram exhibiting a mass loss of about 13.11% from the onset of heating from 200 [symbol] to 250 [symbol] ; or
(j) combinations thereof.

In some embodiments, the crystalline form is Compound 1 Oxalate salt Type A characterized as having at least one of the following properties:
(a) an X-Ray powder diffraction (XRPD) pattern substantially the same as shown in FIG. 44 as measured using Cu Kα.radiation;
(b) an X-Ray powder diffraction (XRPD) pattern with peaks at 11.8±0.2° 2θ, 19.7±0.2° 2θ, and 21.5±0.2° 2θ as measured using Cu Kα.radiation;
(c) a Differential Scanning calorimetry (DSC) thermogram substantially the same as shown in FIG. 45;
(d) a Thermogravimetric Thermal Analysis (TGA) thermogram substantially the same as shown in FIG. 45; or
(e) combinations thereof.

In some embodiments, the crystalline form is Compound 1 Oxalate salt Type A characterized as having at least one of the following properties:
(a) an X-Ray powder diffraction (XRPD) pattern with peaks at 11.8±0.2° 2θ, 19.7±0.2° 2θ, and 21.5±0.2° 2θ as measured using Cu Kα.radiation;
(b) a Differential Scanning calorimetry (DSC) thermogram with an exothermic peak having a peak temperature at about 174.3 [symbol] ;
(c) a Differential Scanning calorimetry (DSC) thermogram with an endothermic peak having a peak temperature at about 251.0 [symbol] ;
(d) a Thermogravimetric Thermal Analysis (TGA) thermogram exhibiting a mass loss of about 5.97% from the onset of heating up to approximately 150.0 [symbol] ;
(e) a Thermogravimetric Thermal Analysis (TGA) thermogram exhibiting a mass loss of about 10.58% from the onset of heating from 150.0 [symbol] to 200 [symbol] ;
(f) a Thermogravimetric Thermal Analysis (TGA) thermogram exhibiting a mass loss of about 13.11% from the onset of heating from 200 [symbol] to 250 [symbol] ; or
(g) combinations thereof.

In some embodiments of Compound 1 Oxalate salt Type A, the crystalline form has an X-ray powder diffraction (XRPD) pattern substantially the same as shown in FIG. 44 as measured using Cu Kα.radiation.

In some embodiments of Compound 1 Oxalate salt Type A, the crystalline form has an X-ray powder diffraction (XRPD) pattern with peaks found in Table 18 as measured using Cu Kα.radiation.

In some embodiments of Compound 1 Oxalate salt Type A, the crystalline form has an X-ray powder diffraction (XRPD) pattern with peaks at 11.8±0.2° 2θ, 19.7±0.2° 2θ, and 21.5±0.2° 2θ as measured using Cu Kα.radiation.

In some embodiments of Compound 1 Oxalate salt Type A, the X-ray powder diffraction (XRPD) pattern further comprises peaks at 7.0±0.2° 2θ, 12.8±0.2° 2θ, and 17.6±0.2° 2θ as measured using Cu Kα.radiation.

In some embodiments of Compound 1 Oxalate salt Type A, the X-ray powder diffraction (XRPD) pattern further comprises peaks at 22.9±0.2° 2θ and 24.7±0.2° 2θ as measured using Cu Kα.radiation.

In some embodiments of Compound 1 Oxalate salt Type A, the crystalline form has an X-ray powder diffraction (XRPD) pattern with peaks at 7.0±0.2° 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the crystalline form has an X-ray powder diffraction (XRPD) pattern with at least two peaks at 7.0±0.2° 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the crystalline form has an X-ray powder diffraction (XRPD) pattern with at least three peaks at 7.0±0.2° 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the crystalline form has an X-ray powder diffraction (XRPD) pattern with at least four peaks at 7.0±0.2° 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the crystalline form has an X-ray powder diffraction (XRPD) pattern with at least five peaks at 7.0±0.2° 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the crystalline form has an X-ray powder diffraction (XRPD) pattern with at least six peaks at 7.0=±0. [symbol] 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the crystalline form has an X-ray powder diffraction (XRPD) pattern with at least seven peaks at 7.0±0.2° 2θ, 11.8±0.2° 2θ, 12.8±0.2° 2θ, 17.6±0.2° 2θ, 19.7±0.2° 2θ, 21.5±0.2° 2θ, 22.9±0.2° 2θ, and 24.7±0.2° 2θ as measured using Cu Kα.radiation.

In some embodiments of Compound 1 Oxalate salt Type A, the Differential Scanning calorimetry (DSC) thermogram is substantially the same as shown in FIG. 45;

In some embodiments of Compound 1 Oxalate salt Type A, the Differential Scanning calorimetry (DSC) thermogram has an exothermic peak having a peak temperature at about 174.3 [symbol] .

In some embodiments of Compound 1 Oxalate salt Type A, the Differential Scanning calorimetry (DSC) thermogram has an endothermic peak having a peak temperature at about 251.0 [symbol] .

In some embodiments of Compound 1 Oxalate salt Type A, the Thermogravimetric Thermal Analysis (TGA) thermogram is substantially the same as shown in FIG. 45.

In some embodiments of Compound 1 Oxalate salt Type A, the Thermogravimetric Thermal Analysis (TGA) thermogram exhibits a mass loss of about 5.97% from the onset of heating up to approximately 150.0 [symbol] .

In some embodiments of Compound 1 Oxalate salt Type A, the Thermogravimetric Thermal Analysis (TGA) thermogram exhibits a mass loss of about 10.58% from the onset of heating from 150.0 [symbol] to 200 [symbol] .

In some embodiments of Compound 1 Oxalate salt Type A, the Thermogravimetric Thermal Analysis (TGA) thermogram exhibits a mass loss of about 13.11% from the onset of heating from 200 [symbol] to 250 [symbol] .

In some embodiments of Compound 1 Oxalate salt Type A, the molar ratio of oxalic acid to Compound 1 freebase is about 0.9:1.

TABLE 18
X-Ray Powder Diffraction peaks of Oxalate Type A

| Pos. [°2θ] | Rel. Int. [%] |
|---|---|
| 7.00 | 10.66 |
| 11.76 | 100.00 |
| 12.76 | 11.14 |
| 14.71 | 6.91 |
| 16.97 | 8.18 |
| 17.62 | 15.18 |
| 18.19 | 10.36 |
| 18.68 | 3.14 |
| 19.67 | 16.59 |
| 20.09 | 3.52 |
| 21.03 | 11.39 |
| 21.50 | 39.80 |
| 22.38 | 3.39 |
| 22.86 | 37.96 |
| 23.50 | 2.26 |
| 24.41 | 10.72 |
| 24.72 | 21.70 |
| 24.99 | 10.05 |
| 25.43 | 10.31 |
| 25.63 | 6.62 |
| 26.72 | 9.66 |
| 27.30 | 3.23 |
| 28.18 | 14.60 |
| 29.01 | 4.34 |
| 30.02 | 7.86 |
| 31.20 | 5.02 |
| 31.98 | 2.66 |
| 32.74 | 1.75 |
| 33.27 | 1.97 |
| 36.00 | 1.17 |
| 37.98 | 1.00 |
| 38.83 | 1.60 |

### Compound 1 Oxalate Salt Type B

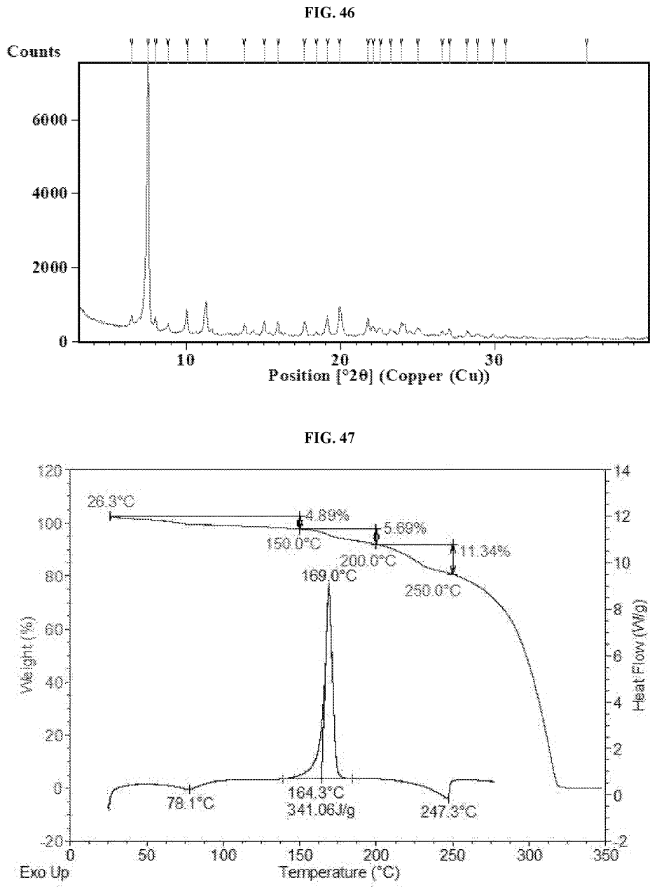

Disclosed herein is Compound 1 Oxalate salt Type B. In some embodiments, the crystalline form is Compound 1 Oxalate salt Type B characterized as having at least one of the following properties:
(a) an X-Ray powder diffraction (XRPD) pattern substantially the same as shown in FIG. 46 as measured using Cu Kα.radiation;
(b) an X-Ray powder diffraction (XRPD) pattern with peaks at 7.5±0.2° 2θ, 10.0±0.2° 2θ, and 11.3±0.2° 2θ as measured using Cu Kα.radiation;
(c) a Differential Scanning calorimetry (DSC) thermogram substantially the same as shown in FIG. 47;
(d) a Differential Scanning calorimetry (DSC) thermogram with an exothermic peak having a peak temperature at about 169.0 [symbol] ;
(e) a Differential Scanning calorimetry (DSC) thermogram with an endothermic peak having a peak temperature at about 78.1 [symbol] ;
(f) a Differential Scanning calorimetry (DSC) thermogram with an endothermic peak having a peak temperature at about 247.3 [symbol] ;
(g) a Thermogravimetric Thermal Analysis (TGA) thermogram substantially the same as shown in FIG. 47;
(h) a Thermogravimetric Thermal Analysis (TGA) thermogram exhibiting a mass loss of about 4.89% from the onset of heating up to approximately 150.0 [symbol] ;
(i) a Thermogravimetric Thermal Analysis (TGA) thermogram exhibiting a mass loss of about 5.69% from the onset of heating from 150.0 [symbol] to 200 [symbol] ;
(j) a Thermogravimetric Thermal Analysis (TGA) thermogram exhibiting a mass loss of about 11.34% from the onset of heating from 200 [symbol] to 250 [symbol] ; or
(k) combinations thereof.

In some embodiments, the crystalline form is Compound 1 Oxalate salt Type B characterized as having at least one of the following properties:
(a) an X-Ray powder diffraction (XRPD) pattern substantially the same as shown in FIG. 46 as measured using Cu Kα.radiation;
(b) an X-Ray powder diffraction (XRPD) pattern with peaks at 7.5±0.2° 2θ, 10.0±0.2° 2θ, and 11.3±0.2° 2θ as measured using Cu Kα.radiation;
(c) a Differential Scanning calorimetry (DSC) thermogram substantially the same as shown in FIG. 47;
(d) a Thermogravimetric Thermal Analysis (TGA) thermogram substantially the same as shown in FIG. 47; or
(e) combinations thereof.

In some embodiments, the crystalline form is Compound 1 Oxalate salt Type B characterized as having at least one of the following properties:
(a) an X-Ray powder diffraction (XRPD) pattern with peaks at 7.5±0.2° 2θ, 10.0±0.2° 2θ, and 11.3±0.2° 2θ as measured using Cu Kα.radiation;
(b) a Differential Scanning calorimetry (DSC) thermogram with an exothermic peak having a peak temperature at about 169.0 [symbol] ;
(c) a Differential Scanning calorimetry (DSC) thermogram with an endothermic peak having a peak temperature at about 78.1 [symbol] ;
(d) a Differential Scanning calorimetry (DSC) thermogram with an endothermic peak having a peak temperature at about 247.3 [symbol] ;
(e) a Thermogravimetric Thermal Analysis (TGA) thermogram exhibiting a mass loss of about 4.89% from the onset of heating up to approximately 150.0 [symbol] ;
(f) a Thermogravimetric Thermal Analysis (TGA) thermogram exhibiting a mass loss of about 5.69% from the onset of heating from 150.0° C. to 200 [symbol] ;
(g) a Thermogravimetric Thermal Analysis (TGA) thermogram exhibiting a mass loss of about 11.34% from the onset of heating from 200 [symbol] to 250 [symbol] ; or
(h) combinations thereof.

In some embodiments of Compound 1 Oxalate salt Type B, the crystalline form has an X-ray powder diffraction (XRPD) pattern substantially the same as shown in FIG. 46 as measured using Cu Kα.radiation.

In some embodiments of Compound 1 Oxalate salt Type B, the crystalline form has an X-ray powder diffraction (XRPD) pattern with peaks found in Table 19 as measured using Cu Kα.radiation.

In some embodiments of Compound 1 Oxalate salt Type B, the crystalline form has an X-ray powder diffraction (XRPD) pattern with peaks at 7.5±0.2° 2θ, 10.0±0.2° 2θ, and 11.3±0.2° 2θ as measured using Cu Kα.radiation.

In some embodiments of Compound 1 Oxalate salt Type B, the X-ray powder diffraction (XRPD) pattern further comprises peaks at 8.0±0.2° 2θ, 19.1±0.2° 2θ, and 20.0±0.2° 2θ as measured using Cu Kα.radiation.

In some embodiments of Compound 1 Oxalate salt Type B, the crystalline form has an X-ray powder diffraction (XRPD) pattern with peaks at 7.5±0.2° 2θ, 8.0±0.2° 2θ, 10.0±0.2° 2θ, 11.3±0.2° 2θ, 19.1±0.2° 2θ, and 20.0±0.2° 2θ as measured using Cu Kα.radiation.

In some embodiments of Compound 1 Oxalate salt Type B, the crystalline form has an X-ray powder diffraction (XRPD) pattern with at least two peaks at 7.5±0.2° 2θ, 8.0±0.2° 2θ, 10.0±0.2° 2θ, 11.3±0.2° 2θ, 19.1±0.2° 2θ, and 20.0±0.2° 2θ as measured using Cu Kα.radiation.

In some embodiments of Compound 1 Oxalate salt Type B, the crystalline form has an X-ray powder diffraction (XRPD) pattern with at least three peaks at 7.5±0.2° 2θ, 8.0±0.2° 2θ, 10.0±0.2° 2θ, 11.3±0.2° 2θ, 19.1±0.2° 2θ, and 20.0±0.2° 2θ as measured using Cu Kα.radiation.

In some embodiments of Compound 1 Oxalate salt Type B, the crystalline form has an X-ray powder diffraction (XRPD) pattern with at least four peaks at 7.5±0.2° 2θ, 8.0±0.2° 2θ, 10.0±0.2° 2θ, 11.3±0.2° 2θ, 19.1±0.2° 2θ, and 20.0±0.2° 2θ as measured using Cu Kα.radiation.

In some embodiments of Compound 1 Oxalate salt Type B, the crystalline form has an X-ray powder diffraction (XRPD) pattern with at least five peaks at 7.5±0.2° 2θ, 8.0±0.2° 2θ, 10.0±0.2° 2θ, 11.3±0.2° 2θ, 19.1±0.2° 2θ, and 20.0±0.2° 2θ as measured using Cu Kα.radiation.

In some embodiments of Compound 1 Oxalate salt Type B, the Differential Scanning calorimetry (DSC) thermogram substantially the same as shown in FIG. 47.

In some embodiments of Compound 1 Oxalate salt Type B, the Differential Scanning calorimetry (DSC) thermogram with an exothermic peak having a peak temperature at about 169.0 [symbol] .

In some embodiments of Compound 1 Oxalate salt Type B, the Differential Scanning calorimetry (DSC) thermogram with an endothermic peak having a peak temperature at about 78.1 [symbol] .

In some embodiments of Compound 1 Oxalate salt Type B, the Differential Scanning calorimetry (DSC) thermogram with an endothermic peak having a peak temperature at about 247.3 [symbol] .

In some embodiments of Compound 1 Oxalate salt Type B, the Thermogravimetric Thermal Analysis (TGA) thermogram substantially the same as shown in FIG. 47.

In some embodiments of Compound 1 Oxalate salt Type B, the Thermogravimetric Thermal Analysis (TGA) thermogram exhibiting a mass loss of about 4.89% from the onset of heating up to approximately 150.0 [symbol] .

In some embodiments of Compound 1 Oxalate salt Type B, the Thermogravimetric Thermal Analysis (TGA) thermogram exhibiting a mass loss of about 5.69% from the onset of heating from 150.0 [symbol] to 200 [symbol] .

In some embodiments of Compound 1 Oxalate salt Type B, the Thermogravimetric Thermal Analysis (TGA) thermogram exhibiting a mass loss of about 11.34% from the onset of heating from 200 [symbol] to 250 [symbol] .

In some embodiments of Compound 1 Oxalate salt Type B, the molar ratio of oxalic acid to Compound 1 freebase is about 0.6:1.

TABLE 19
X-Ray Powder Diffraction peaks of Oxalate Type B

| Pos. [°2θ] | Rel. Int. [%] |
|---|---|
| 6.46 | 4.45 |
| 7.52 | 100.00 |
| 8.01 | 5.08 |
| 8.80 | 3.11 |
| 10.04 | 9.23 |
| 11.29 | 12.48 |
| 13.75 | 4.07 |
| 15.06 | 5.54 |
| 15.95 | 4.63 |
| 17.65 | 5.45 |
| 18.43 | 1.26 |
| 19.14 | 7.49 |
| 19.94 | 11.32 |
| 21.77 | 6.85 |
| 22.11 | 3.65 |
| 22.58 | 2.86 |
| 23.24 | 2.67 |
| 23.93 | 4.81 |
| 25.01 | 3.39 |
| 26.58 | 2.28 |
| 27.08 | 3.06 |
| 28.20 | 3.03 |
| 28.87 | 1.52 |
| 29.88 | 1.65 |
| 30.69 | 1.11 |
| 35.93 | 0.85 |

### Compound 1 Esylate Salt Type A

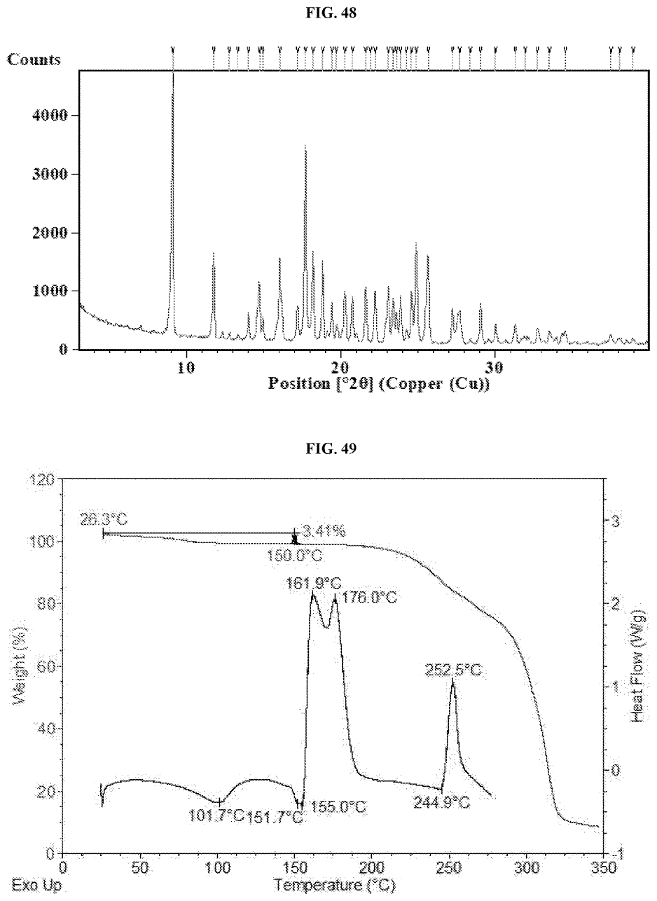

Disclosed herein is Compound 1 Esylate salt Type A. In some embodiments, the crystalline form is Compound 1 Esylate salt Type A characterized as having at least one of the following properties:
(a) an X-Ray powder diffraction (XRPD) pattern substantially the same as shown in FIG. 48 as measured using Cu Kα.radiation;
(b) an X-Ray powder diffraction (XRPD) pattern with peaks at 9.1±0.2° 2θ, 17.7±0.2° 2θ, and 18.2±0.2° 2θ as measured using Cu Kα.radiation;
(c) a Differential Scanning calorimetry (DSC) thermogram substantially the same as shown in FIG. 49;
(d) a Differential Scanning calorimetry (DSC) thermogram with an exothermic peak having a peak temperature at about 161.9 [symbol] ;
(e) a Differential Scanning calorimetry (DSC) thermogram with an exothermic peak having a peak temperature at about 176.0 [symbol] ;
(f) a Differential Scanning calorimetry (DSC) thermogram with an exothermic peak having a peak temperature at about 152.5 [symbol] ;
(g) a Thermogravimetric Thermal Analysis (TGA) thermogram substantially the same as shown in FIG. 49;
(h) a Thermogravimetric Thermal Analysis (TGA) thermogram exhibiting a mass loss of about 3.41% from the onset of heating up to approximately 150.0 [symbol] ; or
(i) combinations thereof.

In some embodiments, the crystalline form is Compound 1 Esylate salt Type A characterized as having at least one of the following properties:
(a) an X-Ray powder diffraction (XRPD) pattern substantially the same as shown in FIG. 48 as measured using Cu Kα.radiation;
(b) an X-Ray powder diffraction (XRPD) pattern with peaks at 9.1±0.2° 2θ, 17.7±0.2° 2θ, and 18.2±0.2° 2θ as measured using Cu Kα.radiation;
(c) a Differential Scanning calorimetry (DSC) thermogram substantially the same as shown in FIG. 49;
(d) a Thermogravimetric Thermal Analysis (TGA) thermogram substantially the same as shown in FIG. 49; or
(e) combinations thereof.

In some embodiments, the crystalline form is Compound 1 Esylate salt Type A characterized as having at least one of the following properties:
(a) an X-Ray powder diffraction (XRPD) pattern with peaks at 9.1±0.2° 2θ, 17.7±0.2° 2θ, and 18.2±0.2° 2θ as measured using Cu Kα.radiation;
(b) a Differential Scanning calorimetry (DSC) thermogram with an exothermic peak having a peak temperature at about 161.9 [symbol] ;
(c) a Differential Scanning calorimetry (DSC) thermogram with an exothermic peak having a peak temperature at about 176.0 [symbol] ;
(d) a Differential Scanning calorimetry (DSC) thermogram with an exothermic peak having a peak temperature at about 152.5 [symbol] ;
(e) a Thermogravimetric Thermal Analysis (TGA) thermogram exhibiting a mass loss of about 3.41% from the onset of heating up to approximately 150.0 [symbol] ; or
(f) combinations thereof.

In some embodiments of Compound 1 Esylate salt Type A, the crystalline form has an X-ray powder diffraction (XRPD) pattern substantially the same as shown in FIG. 48 as measured using Cu Kα.radiation.

In some embodiments of Compound 1 Esylate salt Type A, the crystalline form has an X-ray powder diffraction (XRPD) pattern with peaks found in Table 20 as measured using Cu Kα.radiation.

In some embodiments of Compound 1 Esylate salt Type A, the crystalline form has an X-ray powder diffraction (XRPD) pattern with peaks at 9.1±0.2° 2θ, 17.7±0.2° 2θ, and 18.2±0.2° 2θ as measured using Cu Kα.radiation.

In some embodiments of Compound 1 Esylate salt Type A, the X-ray powder diffraction (XRPD) pattern further comprises peaks at 11.8±0.2° 2θ, 14.7±0.2° 2θ, and 16.0±0.2° 2θ as measured using Cu Kα.radiation.

In some embodiments, the X-ray powder diffraction (XRPD) pattern further comprises peaks at 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with peaks at 9.1±0.2° 2θ, 11.8±0.2° 2θ, 14.7±0.2° 2θ, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least two peaks at 9.1±0.2° 2θ, 11.8±0.2° 2θ, 14.7±0.2° 2θ, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least three peaks at 9.1±0.2° 2θ, 11.8±0.2° 2θ, 14.7±0.2° 20, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least four peaks at 9.1±0.2° 2θ, 11.8±0.2° 2θ, 14.7±0.2° 2θ, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least five peaks at 9.1±0.2° 2θ, 11.8±0.2° 2θ, 14.7±0.2° 2θ, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least six peaks at 9.1±0.2° 2θ, 11.8±0.2° 2θ, 14.7±0.2° 2θ, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least seven peaks at 9.1±0.2° 2θ, 11.8±0.2° 2θ, 14.7±0.2° 20, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the crystalline form has an X-ray powder diffraction (XRPD) pattern with at least eight peaks at 9.1±0.2° 2θ, 11.8±0.2° 2θ, 14.7±0.2° 2θ, 16.0±0.2° 2θ, 17.7±0.2° 2θ, 18.2±0.2° 2θ, 18.8±0.2° 2θ, 20.3±0.2° 2θ, and 21.6±0.2° 2θ as measured using Cu Kα.radiation.

In some embodiments of Compound 1 Esylate salt Type A, the Differential Scanning calorimetry (DSC) thermogram substantially the same as shown in FIG. 49.

In some embodiments of Compound 1 Esylate salt Type A, the Differential Scanning calorimetry (DSC) thermogram with an exothermic peak having a peak temperature at about 161.9 [symbol] .

In some embodiments of Compound 1 Esylate salt Type A, the Differential Scanning calorimetry (DSC) thermogram with an exothermic peak having a peak temperature at about 176.0 [symbol] .

In some embodiments of Compound 1 Esylate salt Type A, the Differential Scanning calorimetry (DSC) thermogram with an exothermic peak having a peak temperature at about 152.5 [symbol] .

In some embodiments of Compound 1 Esylate salt Type A, the Thermogravimetric Thermal Analysis (TGA) thermogram substantially the same as shown in FIG. 49.

In some embodiments of Compound 1 Esylate salt Type A, the Thermogravimetric Thermal Analysis (TGA) thermogram exhibiting a mass loss of about 3.41% from the onset of heating up to approximately 150.0 [symbol] .

In some embodiments of Compound 1 Esylate salt Type A, the molar ratio of ethanesulfonic acid to Compound 1 freebase is about 1:1.

TABLE 20
X-Ray Powder Diffraction peaks of Esylate Type A

| Pos. [°2θ] | Rel. Int. [%] |
|---|---|
| 9.10 | 100.00 |
| 11.77 | 33.20 |
| 12.77 | 2.24 |
| 13.33 | 1.46 |
| 14.04 | 10.00 |
| 14.73 | 22.26 |
| 14.97 | 8.98 |
| 16.05 | 31.69 |
| 17.20 | 13.42 |
| 17.71 | 74.06 |
| 18.21 | 34.25 |
| 18.84 | 30.72 |
| 19.43 | 14.73 |
| 19.71 | 6.11 |
| 20.27 | 18.72 |
| 20.77 | 16.85 |
| 21.63 | 20.76 |
| 21.92 | 0.28 |
| 22.24 | 19.49 |
| 23.09 | 21.32 |
| 23.39 | 16.80 |
| 23.63 | 11.53 |
| 23.89 | 17.90 |
| 24.26 | 4.86 |
| 24.60 | 19.41 |
| 24.89 | 38.31 |
| 25.68 | 32.64 |
| 27.24 | 13.64 |
| 27.72 | 12.58 |
| 28.42 | 1.74 |
| 29.07 | 15.46 |
| 30.04 | 7.41 |
| 31.32 | 7.57 |
| 31.98 | 1.98 |
| 32.74 | 5.62 |
| 33.53 | 4.72 |
| 34.57 | 4.37 |
| 37.53 | 3.29 |
| 38.09 | 1.90 |
| 38.97 | 0.96 |

### Compound 1 Esylate Salt Type B

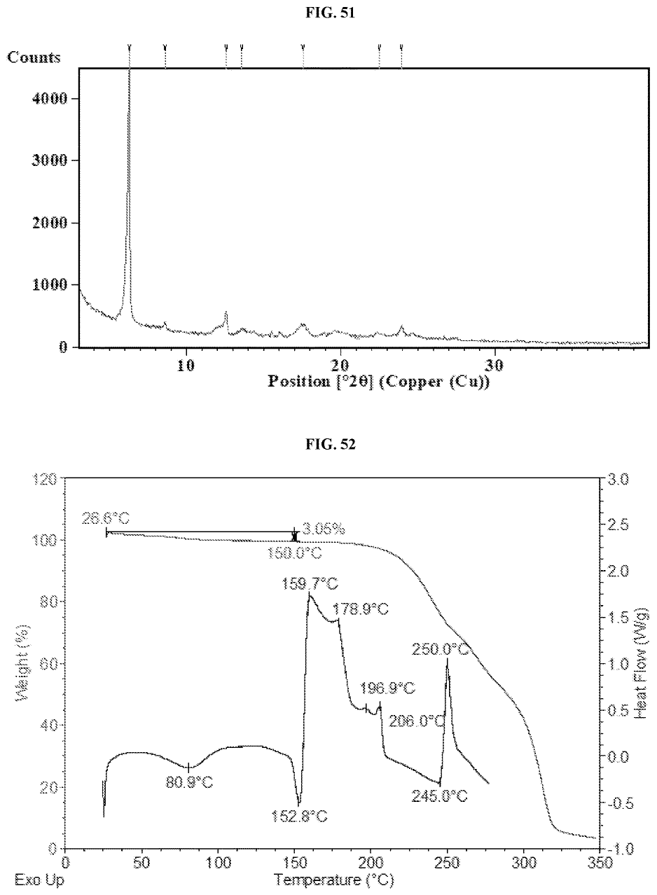

Disclosed herein is Compound 1 Esylate salt Type B. In some embodiments, the crystalline form is Compound 1 Esylate salt Type B characterized as having at least one of the following properties:
(a) an X-Ray powder diffraction (XRPD) pattern substantially the same as shown in FIG. 51 as measured using Cu Kα.radiation;
(b) an X-Ray powder diffraction (XRPD) pattern with peaks at 6.3±0.2° 2θ, 12.6±0.2° 2θ, and 17.6±0.2° 2θ as measured using Cu Kα.radiation;
(c) a Differential Scanning calorimetry (DSC) thermogram substantially the same as shown in FIG. 52;
(d) a Differential Scanning calorimetry (DSC) thermogram with an endothermic peak having a peak temperature at about 80.9 [symbol] ;
(e) a Differential Scanning calorimetry (DSC) thermogram with an endothermic peak having a peak temperature at about 152.8 [symbol] ;
(f) a Differential Scanning calorimetry (DSC) thermogram with an exothermic peak having a peak temperature at about 159.7 [symbol] ;
(g) a Differential Scanning calorimetry (DSC) thermogram with an exothermic peak having a peak temperature at about 178.9 [symbol] ;
(h) a Differential Scanning calorimetry (DSC) thermogram with an exothermic peak having a peak temperature at about 196.9 [symbol] ;
(i) a Differential Scanning calorimetry (DSC) thermogram with an exothermic peak having a peak temperature at about 206.0 [symbol] ;
(j) a Differential Scanning calorimetry (DSC) thermogram with an exothermic peak having a peak temperature at about 250.0 [symbol] ;
(k) a Thermogravimetric Thermal Analysis (TGA) thermogram substantially the same as shown in FIG. 52;
(l) a Thermogravimetric Thermal Analysis (TGA) thermogram exhibiting a mass loss of about 3.05% from the onset of heating up to approximately 150.0 [symbol] ; or
(m) combinations thereof.

In some embodiments, the crystalline form is Compound 1 Esylate salt Type B characterized as having at least one of the following properties:
(a) an X-Ray powder diffraction (XRPD) pattern substantially the same as shown in FIG. 51 as measured using Cu Kα.radiation;
(b) an X-Ray powder diffraction (XRPD) pattern with peaks at 6.3±0.2° 2θ, 12.6±0.2° 2θ, and 17.6±0.2° 2θ as measured using Cu Kα.radiation;
(c) a Differential Scanning calorimetry (DSC) thermogram substantially the same as shown in FIG. 52;
(d) a Thermogravimetric Thermal Analysis (TGA) thermogram substantially the same as shown in FIG. 52; or
(e) combinations thereof.

In some embodiments, the crystalline form is Compound 1 Esylate salt Type B characterized as having at least one of the following properties:
(a) an X-Ray powder diffraction (XRPD) pattern with peaks at 6.3±0.2° 2θ, 12.6±0.2° 2θ, and 17.6±0.2° 2θ as measured using Cu Kα.radiation;
(b) a Differential Scanning calorimetry (DSC) thermogram with an endothermic peak having a peak temperature at about 80.9 [symbol] ;
(c) a Differential Scanning calorimetry (DSC) thermogram with an endothermic peak having a peak temperature at about 152.8 [symbol]
(d) a Differential Scanning calorimetry (DSC) thermogram with an exothermic peak having a peak temperature at about 159.7 [symbol] ;
(e) a Differential Scanning calorimetry (DSC) thermogram with an exothermic peak having a peak temperature at about 178.9 [symbol] ;
(f) a Differential Scanning calorimetry (DSC) thermogram with an exothermic peak having a peak temperature at about 196.9 [symbol] ;
(g) a Differential Scanning calorimetry (DSC) thermogram with an exothermic peak having a peak temperature at about 206.0 [symbol] ;
(h) a Differential Scanning calorimetry (DSC) thermogram with an exothermic peak having a peak temperature at about 250.0 [symbol] ;
(i) a Thermogravimetric Thermal Analysis (TGA) thermogram exhibiting a mass loss of about 3.05% from the onset of heating up to approximately 150.0 [symbol] ; or
(j) combinations thereof.

In some embodiments of Compound 1 Esylate salt Type B, the crystalline form has an X-ray powder diffraction (XRPD) pattern substantially the same as shown in FIG. 51 as measured using Cu Kα.radiation.

In some embodiments of Compound 1 Esylate salt Type B, the crystalline form has an X-ray powder diffraction (XRPD) pattern with peaks found in Table 21 as measured using Cu Kα.radiation.

In some embodiments of Compound 1 Esylate salt Type B, the crystalline form has an X-ray powder diffraction (XRPD) pattern with peaks at 6.3±0.2° 2θ, 12.6±0.2° 2θ, and 17.6±0.2° 2θ as measured using Cu Kα.radiation.

In some embodiments of Compound 1 Esylate salt Type B, the X-ray powder diffraction (XRPD) pattern further comprises peaks at 8.6±0.2° 2θ and 23.9±0.2° 2θ as measured using Cu Kα.radiation.

In some embodiments of Compound 1 Esylate salt Type B, the crystalline form has an X-ray powder diffraction (XRPD) pattern with peaks at 6.3±0.2° 2θ, 8.6±0.2° 2θ, 12.6±0.2° 2θ, 17.6±0.2° 2θ, and 23.9±0.2° 2θ as measured using Cu Kα.radiation.

In some embodiments of Compound 1 Esylate salt Type B, the crystalline form has an X-ray powder diffraction (XRPD) pattern with at least two peaks at 6.3±0.2° 2θ, 8.6±0.2° 2θ, 12.6±0.2° 2θ, 17.6±0.2° 2θ, and 23.9±0.2° 2θ as measured using Cu Kα.radiation.

In some embodiments of Compound 1 Esylate salt Type B, the crystalline form has an X-ray powder diffraction (XRPD) pattern with at least three peaks at 6.3±0.2° 2θ, 8.6±0.2° 2θ, 12.6±0.2° 2θ, 17.6±0.2° 2θ, and 23.9±0.2° 2θ as measured using Cu Kα.radiation.

In some embodiments of Compound 1 Esylate salt Type B, the crystalline form has an X-ray powder diffraction (XRPD) pattern with at least four peaks at 6.3±0.2° 2θ, 8.6±0.2° 2θ, 12.6±0.2° 2θ, 17.6±0.2° 2θ, and 23.9±0.2° 2θ as measured using Cu Kα.radiation.

In some embodiments of Compound 1 Esylate salt Type B, the Differential Scanning calorimetry (DSC) thermogram is substantially the same as shown in FIG. 52.

In some embodiments of Compound 1 Esylate salt Type B, the Differential Scanning calorimetry (DSC) thermogram has an endothermic peak having a peak temperature at about 80.9 [symbol] .

In some embodiments of Compound 1 Esylate salt Type B, the Differential Scanning calorimetry (DSC) thermogram has an endothermic peak having a peak temperature at about 152.8 [symbol] .

In some embodiments of Compound 1 Esylate salt Type B, the Differential Scanning calorimetry (DSC) thermogram has an exothermic peak having a peak temperature at about 159.7 [symbol] .

In some embodiments of Compound 1 Esylate salt Type B, the Differential Scanning calorimetry (DSC) thermogram has an exothermic peak having a peak temperature at about 178.9 [symbol] .

In some embodiments of Compound 1 Esylate salt Type B, the Differential Scanning calorimetry (DSC) thermogram has an exothermic peak having a peak temperature at about 196.9 [symbol] .

In some embodiments of Compound 1 Esylate salt Type B, the Differential Scanning calorimetry (DSC) thermogram has an exothermic peak having a peak temperature at about 206.0 [symbol] .

In some embodiments of Compound 1 Esylate salt Type B, the Differential Scanning calorimetry (DSC) thermogram has an exothermic peak having a peak temperature at about 250.0 [symbol] .

In some embodiments of Compound 1 Esylate salt Type B, the Thermogravimetric Thermal Analysis (TGA) thermogram is substantially the same as shown in FIG. 52.

In some embodiments of Compound 1 Esylate salt Type B, the Thermogravimetric Thermal Analysis (TGA) thermogram exhibits a mass loss of about 3.05% from the onset of heating up to approximately 150.0 [symbol] .

In some embodiments of Compound 1 Esylate salt Type B, the molar ratio of ethanesulfonic acid to Compound 1 freebase is about 1:1.

TABLE 21
X-Ray Powder Diffraction peaks of Esylate Type B

| Pos. [°2θ] | Rel. Int. [%] |
|---|---|
| 6.29 | 100.00 |
| 8.61 | 2.80 |
| 12.57 | 8.65 |
| 13.60 | 2.52 |
| 17.55 | 4.83 |
| 22.49 | 1.94 |
| 23.94 | 4.84 |

### Method of Treatment

Disclosed herein is a method of treating a disease in which inhibition of MAT2A is beneficial, the method comprising administering a crystalline forms disclosed herein. In some embodiments, the method comprises administering a pharmaceutical composition comprising a crystalline forms disclosed herein.

Disclosed herein is a method of treating a disease or disorder associated with MAT2A, the method comprising administering to the subject a crystalline forms disclosed herein. In some embodiments, the method comprises administering a pharmaceutical composition comprising a crystalline forms disclosed herein.

Disclosed herein is a method of treating cancer in a subject, the method comprising administering to the subject a crystalline forms disclosed herein.

In some embodiments, the cancer is a primary leukemia, hematological malignancies, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the cancer is liver cancer, colon cancer, pancreatic cancer, prostate cancer, lung cancer, breast cancer, or T cell leukemia.

In some embodiments, the cancer is liver cancer, colon cancer, pancreatic cancer, prostate cancer, lung cancer, breast cancer, gastrointestinal stromal tumor, biliary tract cancer, acute lymphoblastic leukemia (ALL) B-lineage, lymphoma, or T cell leukemia.

In some embodiments, the cancer is a MTAP-deficient cancer. In some embodiments, the MTAP-deficient cancer is a primary leukemia, hematological malignancies, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In other embodiments, the MTAP-deficient cancer is MTAP-deficient lung cancer, MTAP-deficient pancreatic cancer, MTAP-deficient esophageal cancer, MTAP-deficient colorectal cancer, MTAP-deficient kidney cancer, or MTAP-deficient leukemia, such as acute myeloid leukemia (AML).

In some embodiments, the MTAP-deficient cancer is MTAP-deficient lung cancer, such as NSCLC.

In other embodiments, the MTAP-deficient cancer is MTAP-deficient pancreatic cancer, such as PDAC. In some embodiments, the MTAP-deficient cancer is MTAP-deficient esophageal cancer.

In some embodiments, the MTAP-deficient cancer is MTAP-deficient colorectal cancer.

In some embodiments, the MTAP-deficient cancer is MTAP-deficient kidney cancer.

In some embodiments, the MTAP-deficient cancer is MTAP-deficient leukemia, such as acute myeloid leukemia (AML).

In some embodiments, the cancer is a MTAP wild type cancer. In some embodiments, the MTAP wild type cancer is a primary leukemia, hematological malignancy, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the MTAP wild type cancer is MTAP wild type lung cancer, MTAP wild type pancreatic cancer, MTAP wild type esophageal cancer, MTAP wild type colorectal cancer, MTAP wild type kidney cancer, or MTAP wild type leukemia, such as acute myeloid leukemia (AML).

In some embodiments, the MTAP wild type cancer is MTAP wild type lung cancer, such as NSCLC.

In some embodiments, the MTAP wild type cancer is MTAP wild type pancreatic cancer, such as PDAC.

In some embodiments, the MTAP wild type cancer is MTAP wild type esophageal cancer.

In some embodiments, the MTAP wild type cancer is MTAP wild type colorectal cancer.

In some embodiments, the MTAP wild type cancer is MTAP wild type kidney cancer.

In some embodiments, the MTAP wild type cancer is MTAP wild type leukemia, such as acute myeloid leukemia (AML).

In some embodiments, the cancer is a cancer that responds to a reduction in SAM as a result of administering an MAT2A inhibitor. In some embodiments, the cancer that responds to a reduction in SAM as a result of administering an MAT2A inhibitor is a primary leukemia, hematological malignancy, acute myeloid leukemia (AML), glioma, melanoma, pancreatic cancer, non-small cell lung cancer (NSCLC), bladder cancer, kidney cancer, colorectal cancer, esophageal cancer, astrocytoma, osteosarcoma, head and neck cancer, myxoid chondrosarcoma, ovarian cancer, endometrial cancer, breast cancer, soft tissue sarcoma, non-Hodgkin lymphoma, or mesothelioma.

In some embodiments, the cancer that responds to a reduction in SAM as a result of administering an MAT2A inhibitor is lung cancer, pancreatic cancer, esophageal cancer, colorectal cancer, kidney cancer, or leukemia, such as acute myeloid leukemia (AML).

In some embodiments, the cancer that responds to a reduction in SAM as a result of administering an MAT2A inhibitor is lung cancer, such as NSCLC.

In some embodiments, the cancer that responds to a reduction in SAM as a result of administering an MAT2A inhibitor is pancreatic cancer, such as PDAC.

In some embodiments, the cancer that responds to a reduction in SAM as a result of administering an MAT2A inhibitor is esophageal cancer.

In some embodiments, the cancer that responds to a reduction in SAM as a result of administering an MAT2A inhibitor is colorectal cancer.

In some embodiments, the cancer that responds to a reduction in SAM as a result of administering an MAT2A inhibitor is kidney cancer.

In some embodiments, the cancer that responds to a reduction in SAM as a result of administering an MAT2A inhibitor is leukemia, such as acute myeloid leukemia (AML).

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The crystalline forms described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a crystalline form described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

### Definitions

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The terms “administer,” “administering,” “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “about” means within a statistically meaningful range of a value, such as a stated concentration range, time frame, molecular weight, particle size, temperature, or pH. Such a range can be within an order of magnitude, typically within 10%, more typically within 5%, and even more typically within 3% of the indicated value or range. Sometimes, such a range can be within the experimental error typical of standard methods used for the measurement and/or determination of a given value or range. The allowable variation encompassed by the term “about” will depend upon the particular system under study, and can be readily appreciated by one of ordinary skill in the art. Whenever a range is recited within this application, every whole number integer within the range is also contemplated as an embodiment of the disclosure. In the context of the disclosure, when used or whether or not used the word, such as “about,” it means that within a given value or range of 10%, appropriately within 5%, especially within 1%.

If multiple diffraction patterns are available, then assessments of particle statistics (PS) and/or preferred orientation (PO) are possible. Consistency of relative intensity among XRPD patterns from multiple diffractometers indicates good orientation statistics. Alternatively, the observed XRPD pattern may be compared with a calculated XRPD pattern based upon a single crystal structure, if available. Two-dimensional scattering patterns using area detectors can also be used to evaluate PS/PO. If the effects of both PS and PO are determined to be negligible, then the XRPD pattern is representative of the powder average intensity for the sample and prominent peaks may be identified as “Representative Peaks.” In general, the more data collected to determine Representative Peaks, the more confident one can be of the classification of those peaks.

“Characteristic peaks,” to the extent they exist, are a subset of representative peaks and are used to differentiate one crystalline polymorph from another crystalline polymorph (polymorphs being crystalline forms having the same chemical composition). Characteristic peaks are determined by evaluating which representative peaks, if any, are present in one crystalline polymorph of a compound against all other known crystalline polymorphs of that compound to within ═0.2° 2Θ. Not all crystalline polymorphs of a compound necessarily have at least one characteristic peak.

The term “preferred orientation” as used herein refers to an extreme case of non-random distribution of the crystallites of a solid state form. In XRPD, the ideal sample is homogenous and the crystallites are randomly distributed in the bulk solid. In a truly random sample, each possible reflection from a given set of planes will have an equal number of crystallites contributing to it. However, when the solid state form is in a preferred orientation this is not the case. Accordingly, comparing the intensity between a randomly oriented diffraction pattern and a preferred oriented diffraction pattern can look entirely different. Quantitative analysis depending on intensity ratios are greatly distorted by preferred orientation. Careful sample preparation is important for decreasing the incidence of a preferred orientation.

The term “substantially the same,” as used herein to reference a figure is intended to mean that the figure is considered representative of the type and kind of characteristic data that is obtained by a skilled artisan in view of deviations acceptable in the art. Such deviations may be caused by factors related to sample size, sample preparation, particular instrument used, operation conditions, and other experimental condition variations known in the art. For example, one skilled in the art can appreciate that the endotherm onset and peak temperatures as measured by differential scanning calorimetry (DSC) may vary significantly from experiment to experiment. For example, one skilled in the art can readily identify whether two X-ray diffraction patterns or two DSC thermograms are substantially the same. In some embodiments, when characteristic peaks of two X-ray diffraction patterns do not vary more than ±0.2° 2-θ, it is deemed that the X-ray diffraction patterns are substantially the same.

As used herein, the term ‘MTAP-deficient cancer’ refers to a cancer which lacks activity of the metabolic enzyme methylthioadenosine phosphorylase (MTAP). Thus, an MTAP-deficient cancer is a cancer that is associated with a failure to express the MTAP gene, which failure may be attributable to the absence of MTAP gene, the lack of MTAP protein expression, or accumulation of MTAP substrate MTA. In some embodiments the term ‘MTAP-deficient’ is referred to as ‘MTAP-deleted’ and/or ‘MTAP-null’ and thus the three terms may be used interchangeably. For example in some embodiments, ‘MTAP-deleted’ or ‘MTAP-null’ cancer refers to chromosomal loss of the MTAP gene, resulting in full or partial loss of MTAP DNA which prevents expression of functional, full length MTAP protein. In some embodiments a MTAP-deficient cancer is a cancer where the locus of the CDKN2A gene is absent or deleted. In some embodiments, an MTAP-deficient cancer is one in which the MTAP gene has been deleted, lost, or otherwise deactivated. In some embodiments, an MTAP-deficient cancer is a cancer in which the MTAP protein has a reduced function or is functionally impaired as compared to a wild type MTAP gene. Accordingly, in an embodiment of the present disclosure, there is provided a method for treating a MTAP-deficient cancer in a subject, wherein the cancer is characterized by at least one of (i) a reduction or absence of MTAP expression; (ii) absence of the MTAP gene; and (iii) reduced function of MTAP protein, as compared to the corresponding cancers where the MTAP gene and/or protein is present and fully functioning, or as compared to the corresponding cancers with the wild type MTAP gene.

### Examples

The following examples are provided for illustrative purposes only and not to limit the scope of the claims provided herein.

### Instruments and Methods

For XRPD analysis, PANalytical X′pert 3 and Empyrean X-ray powder diffractometers were used. Sample was spread on the middle of a zero-background Si holder. The XRPD parameters used are listed Table 22.

TABLE 22
Parameters for XRPD test

| Model | X′ Pert3/Empyrean |
|---|---|
| X-Ray wavelength | Cu, Kα, |
| | Kα1 (Å): 1.540598, |
| | Kα2 (Å): 1.544426 |
| | Kα2/Kα1 intensity ratio: 0.50 |
| X-Ray tube setting | 45 kV, 40 mA |
| Divergence slit | ⅛° |
| Scan mode | Continuous |
| Scan range (°2TH) | 3-40 |
| Scan step time (s) | 46.7 |
| Step size (°2TH) | 0.0263 |
| Test Time (s) | ~5 min |

TGA data were collected using a Discovery 5500 TGA from TA Instruments and DSC was performed using a Discovery 2500 DSC from TA Instruments. Detailed parameters used are listed in Table 23.

TABLE 23
Parameters for TGA and DSC test

| Parameters | TGA | DSC |
|---|---|---|
| Method | Ramp | Ramp |
| Sample pan | Aluminum, open | Aluminum, crimped |
| Temperature | RT - desired temperature | 25° C. - desired temperature |
| Heating rate | 10° C./min | 10° C./min |
| Purge gas | N₂ | N₂ |

### Example 1: Preparation of Compound 1 Freebase Type A

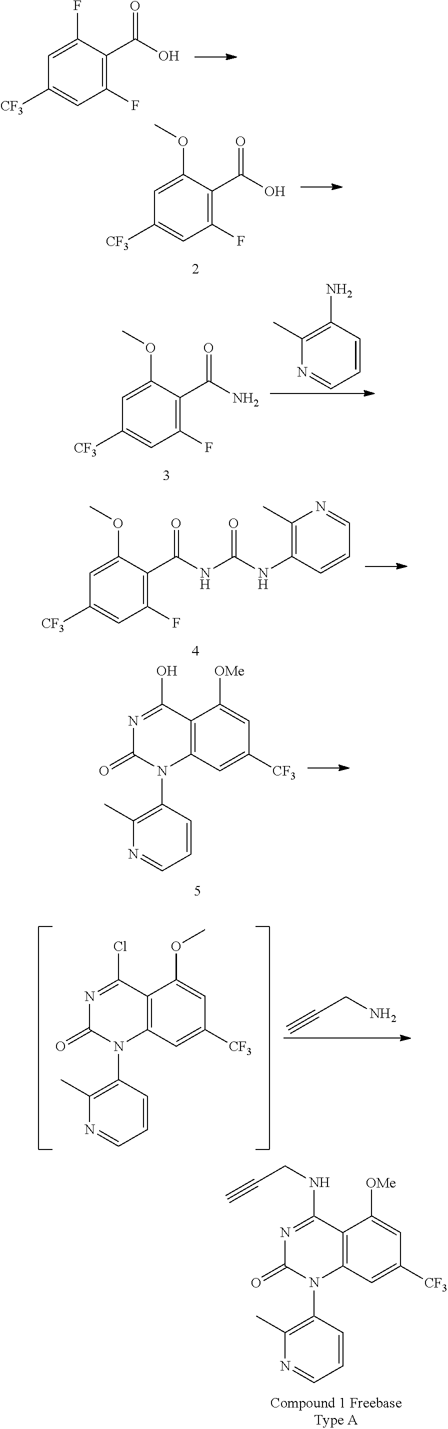

### Step 1: Procedure for Preparation of Compound 2 (2-fluoro-6-methoxy-4-(trifluoromethyl)benzoic Acid)

To a solution of sodium methanolate (358.0 mg, 6.63 mmol) in MeOH (5 mL) were added 2,6-difluoro-4-(trifluoromethyl)benzoic acid (500.0 mg, 2.21 mmol), and the reaction was stirred at 80° C. for 2 hrs. The mixture was then cooled to room temperature and concentrated The residue was diluted with water (10 mL), adjusted pH to 2~3 with aqueous 6.0 M HCl and the mixture was extracted with DCM (15.0 mL×3), the organic layer was washed by brine, dried, and concentrated to afford compound 2 (215.0 mg, 40.8% yield). ¹H NMR (400 MHz, DMSO-d₆) δ 7.37 (d, J=8.8 Hz, 1H), 7.28 (s, 1H), 3.92 (s, 3H);

### Step 2: Procedure for Preparation of Compound 3 (2-fluoro-6-methoxy-4-(trifluoromethyl)benzamide)

Intermediate 2 (80.0 g, 357.0 mmol, 1.00 eq) was mixed in SOCl₂ (600 mL), and then the resulting solution was heated at 80° C. for 2 h. The solution was then cooled to room temperature. Excess SOCl₂ was removed under vacuum, and the residue was dissolved in dioxane (600 mL), treated with NH₄OH (40%, 600 mL). The resulting solution was stirred at 0°p9 C. to room temperature for 1 h. The mixture was concentrated under reduced pressure and diluted with water while stirring until a white solid was precipitated. Then the solid product was filtered and washed with water as to obtain intermediate 3 (70.0 g, 324.6 mmol, 88.6% yield) as a white solid. ¹H NMR (400 MHz, DMSO-d₆) δ 7.96 (s, 1H), 7.71 (s, 1H), 7.31 (d, J=8.8 Hz, 1H), 7.23 (s, 1H), 3.89 (s, 3H).

### Step 3: Procedure for Preparation of Compound 4 (2-fluoro-6-methoxy-N-((2-methylpyridin-3-yl)carbamoyl)-4-(trifluoromethyl)benzamide)

To a stirred suspension of intermediate 3 (22.0 g, 92.7 mmol, 1.0 eq) in dichloroethane (200.0 mL) at room temperature was added oxalyl dichloride (13.0 g, 102.0 mmol, 1.10 eq). The resultant suspension was heated to 80° C. for 1 h. The reaction mixture was allowed to cool to room temperature, 2-methylpyridin-3-amine (20.0 g, 185.4 mmol, 2.00 eq) was added to the reaction mixture (on addition a suspension formed). The resultant reaction mixture was stirred at room temperature for 20 min, then cooled to 0° C. The precipitate was collected by filtration, washed with water, and dried under vacuum to afford intermediate 4 (26.0 g, 70.0 mmol, 75.6% yield) as a faint yellow solid. LCMS:Rt: 1.285 min; MS m/z (ESI): 372.0 [M+H];

### Step 4: Procedure for Preparation of Compound 5 (4-hydroxy-5-methoxy-1-(2-methylpyridin-3-yl)-7-(trifluoromethyl) quinazolin-2 (1H)-one)

KHMDS (154.0 mL, 154.0 mmol, 2.20 eq, 1M in THF) was added to a mixture of intermediate 4 (26.0 g, 70.0 mmol, 1.00 eq) in THF (250.0 mL) at −20° C., and the resulting mixture was allowed to warm to room temperature over 1 h. The reaction mixture was concentrated, diluted with water and adjust pH to 6~7 with aqueous 4M aq. HCl. The precipitate was collected by filtration, washed with water, and dried under vacuum to afford intermediate 5 (20.0 g, 56.9 mmol, 81.3%) as a faint yellow solid.

1H NMR (400 MHz, DMSO-d₆) δ 11.71 (s, 1H), 8.66 (dd, J=4.8, 1.6 Hz, 1H), 7.88 (dd, J=8.0, 1.6 Hz, 1H), 7.52 (dd, J=7.6, 4.8 Hz, 1H), 7.15 (s, 1H), 5.98 (s, 1H), 3.98 (s, 3H), 2.25 (s, 3H).

### Steps 5 and 6: Procedure for Preparation of (5-methoxy-1-(2-methylpyridin-3-yl)-4-(prop-2-yn-1-ylamino)-7-(trifluoromethyl) quinazolin-2 (1H)-one)

To a stirred suspension of 4-hydroxy-5-methoxy-1-(2-methylpyridin-3-yl)-7-(trifluoromethyl) quinazolin-2 (1H)-one (intermediate 5, 100 g, 285 mmol, 1.00 eq) in ACN (1000 mL), DIEA (147 g, 1.14 mol, 4 eq) was added. The mixture was heated and maintained at 50 C., POCl₃ (132 g, 850 mmol, 3.0 eq) was dropped. The solution was heated at 50° C. for 1 h, then cooled to 0-10 [symbol] . A solution of DIEA (147 g, 1.14 mol, 4 eq) and prop-2-yn-1-amine (62 g, 1.13 mmol, 4 eq) in ACN (300 mL) was added. The mixture was stirred at 0-10° C. for 2 h, ACN was removed under vacuum. DCM (500 mL) and Water (200 mL) was added, and separate phase. The organic layer was washed by aq. Na₂CO₃ and brine, dried over anhydrous Na₂SO₄, filtered, and concentrated to provide the crude product. This crude product was slurried with IPA and MTBE (⅓ v/v, 800 mL), filtered and dried under vacuum at 45° C. for 12 hours to afford 5-methoxy-1-(2-methylpyridin-3-yl)-4-(prop-2-yn-1-ylamino)-7-(trifluoromethyl) quinazolin-2 (1H)-one as a yellow solid (60% yield). LCMS:Rt: 1.207 min; MS m/z (ESI): 389.1 [M+H]; ¹H NMR (400 MHz, DMSO-d₆) δ 8.98 (t, J=6.0 Hz, 1H), 8.62 (dd, J=4.8, 1.6 Hz, 1H), 7.77 (dd, J=8.0, 1.2 Hz, 1H), 7.49 (dd, J=8.0, 4.8 Hz, 1H), 7.15 (s, 1H), 6.03 (s, 1H), 4.41-4.27 (m, 2H), 4.13 (s, 3H), 3.16 (t, J=2.4 Hz, 1H), 2.16 (s, 3H).

### Characterization of Compound 1 Freebase Type A

Freebase Type A was characterized by XRPD, TGA, and DSC. XRPD is displayed in FIG. 1 with diffraction peaks in Table 1. TGA and DSC results are shown in FIG. 2. TGA showed a weight loss of 1.19% up to 180 [symbol] , and DSC showed one endothermic peak at 228.4 [symbol] (peak). Considering limited TGA weight loss and neat DSC, freebase Type A was speculated to be an anhydrate.

### Example 2: Pharmacokinetics of Compound 1 Freebase Type A in Male Sprague Dawley Rats

Test compounds was dissolved in 5% DMSO+95% (10% HP-β-CD in Saline) to a concentration of 0.2 mg/mL and given to SD rats (Male, n=3) by intravenous injection (5 mL/kg). Blood samples were collected at 0.083 h, 0.25, 0.5, 1, 2, 4, 8, and 24 h after injection (anticoagulant:K2-EDTA). These tubes were kept in an ice bath prior to centrifugation at approximately 6800 g for 6 minutes at approximately 5° C. Samples were analyzed for the presence of the test compound with an TQ5500 mass spectrometer (AB Sciex) coupled to an ACQUITY UPLC BEH C18 (1.7 μm, 2.1 mm×50 mm) HPLC column. Gradient elution was applied consisting of ultrapure water containing 0.1% formic acid and acetonitrile containing 0.1% formic acid. Standard set of parameters including Area Under the Curve (AUC (0-t) and AUC (0-0)), elimination half-live (T1/2), maximum plasma concentration (Cmax), time to reach maximum plasma concentration (Tmax) will be calculated using non-compartmental analysis modules in Phoenix WinNonlin 7.0 (Pharsight, USA).

TABLE 24

| | Compound 1 | Compound A in |
|---|---|---|
| | Freebase Type A | WO2022256806A1 |
| CL (mL/min/kg) | 0.17 | 7.32 |

### Example 3: Assay for Inhibition of Cellular Proliferation

Test compound impact on cancer cell growth was assessed by treating cancer cells with compound for 5 days and then measuring proliferation using an ATP-based cell proliferation readout (Cell Titer Glo, Promega Corporation).

In a typical assay an isogenic pair of HCT116 human colon carcinoma cell lines which vary only in MTAP deletion status (HCT116 MTAP+/+ and HCT116 MTAP−/−) were plated in 96-well dishes at appropriate cell density. Following 24 hours, cells were then treated with the candidate MAT2A inhibitor. Prior to addition to cells, the compound was first serially diluted in DMSO, typically as a 3-fold serial dilution with 9 dose points.

Compound was then transferred to a working stock plate in cell culture media by adding 3 μl of compound in DMSO to 297 μl of cell culture media. This working stock was then added to cells, by adding 80 μl of working stock to 80 μl of cells in culture media. Following compound addition, cells were incubated at 37 [symbol] /5% CO₂ for 5 days.

To measure inhibition of cellular proliferation, cells were allowed to equilibrate to room temperature for 30 minutes, and were then treated with 40 μl of Cell Titer Glo reagent. The plate was then shaken for 2 minutes to ensure complete mixing and full cell lysis. Luminescent signal of the Cell Titer Glo reagent was then measured using a plate-based luminometer and normalized via an ATP standard curve. This luminescence measure was converted to a proliferation index by subtracting from each datapoint the ATP luminescence measured from a control cell plate that was measured at the time of compound treatment. Compound activity was then represented as a % change in proliferation relative to a within-plate DMSO control.

TABLE 25

| | Compound 1 | Compound A in |
|---|---|---|
| | Freebase Type A | WO2022256806A1 |
| HCT116 MTAP -/- IC50 (nM) | 198.3 | 2008 |
| HCT116 WT IC50 (nM) | 21020 | >30000 |

### Example 4: Preparation of Compound 1 Freebase Type B

About 20 mg of Compound 1 Freebase Type A was suspended in 0.5 mL of IPA/H₂O (85:15, v/v). The sample was stirred at RT for seven days. The suspension was filtered and solid was collected for XRPD analysis. The wet solid was air dried before TGA and DSC analysis.

XRPD pattern is displayed in FIG. 3 with diffraction peaks in Table 2. TGA and DSC curves are displayed in FIG. 4. TGA showed a weight loss of 12.78% up to 150 [symbol] , DSC showed four endothermic peaks at 87.5 [symbol] , 103.8 [symbol] , 226.7 [symbol] (peak) and one exothermic peak at 109.4 [symbol] (peak). Freebase Type B was speculated to be a hydrate.

### Example 5: Preparation of Compound 1 Freebase Type C

Approximate 20 mg Freebase Type A was suspended in 0.5 mL of acetone/H₂O (19:1, v/v). The sample was stirred at RT for five days. The solids were centrifuged and vacuum dried for XRPD, TGA, and DSC analysis.

XRPD is displayed in FIG. 5 with diffraction peaks in Table 3. TGA/DSC results are displayed in FIG. 6. TGA showed step weight loss of 2.62% up to 50.0 [symbol] , 3.39% from 50.0 [symbol] to 70.0 [symbol] and 2.44% from 70.0 [symbol] to 100.0 [symbol] , DSC showed four endothermic peaks at 44.9 [symbol] , 72.5 [symbol] , 89.6 [symbol] , 226.8 [symbol] (peak) and one exothermic peak at 108.7° C. (peak). Freebase Type C was speculated to be a hydrate.

### Example 6: Preparation of Compound 1 HCl Salt Type A

Compound 1 HCl salt Type A was prepared via slurring approximate 20 mg Compound 1 Freebase Type A and equal molar HCl in 0.5 mL acetone/H₂O (19:1, v/v). The suspension was centrifuged and vacuum dried at 50 [symbol] for XRPD, TGA, and DSC analysis.

XRPD and TGA/DSC results are displayed in FIG. 7, Table 4 and FIG. 8. TGA result showed step weight loss of 11.04% up to 100.0 [symbol] and 6.03% from 100 [symbol] to 175 [symbol] , DSC result showed two endothermic peaks at 97.5 [symbol] , 139.2 [symbol] (peak) and one exothermic peak at 170.4 [symbol] (peak). ¹H NMR results showed no solvent was detected. HPLC/IC showed the molar ratio of acid to freebase was 0.6.

### Example 7: Preparation of Compound 1 HCl Salt Type B

Compound 1 HCl salt Type B was prepared via slurring approximate 20 mg Compound 1 Freebase Type A and equal molar HCl in 0.5 mL EtOAc. The suspension was centrifuged and vacuum dried at 50 [symbol] for XRPD, TGA, and DSC analysis.

XRPD and TGA/DSC results are displayed in FIG. 9, Table 5, and FIG. 10. TGA result showed step weight loss of 6.30% up to 120.0 [symbol] and 8.82% from 120 [symbol] to 175 [symbol] , DSC result showed two endothermic peaks at 112.6 [symbol] , 148.7 [symbol] (peak) and one exothermic peak at 166.0° C. (peak). HPLC/IC showed the molar ratio of acid to freebase was 1.0.

### Example 8: Preparation of Compound 1 HCl Salt Type C

Compound 1 HCl salt Type C was prepared via slurring approximate 20 mg Compound 1 Freebase Type A and equal molar HCl in 0.5 mL IPA. The suspension was centrifuged and vacuum dried at 50 [symbol] for XRPD, TGA, and DSC analysis.

XRPD and TGA/DSC results were displayed in FIG. 11, Table 6, and FIG. 12. TGA result showed step weight loss of 7.64% up to 100.0 [symbol] and 5.67% from 100 [symbol] to 150 [symbol] , DSC result showed three endothermic peaks at 75.8 [symbol] , 113.9 [symbol] and 148.4 [symbol] (peak). HPLC/IC showed the molar ratio of acid to freebase was 0.7.

### Example 9: Preparation of Compound 1 Sulfate Salt Type A

Compound 1 Sulfate Type A was obtained via slurry 20 mg Compound 1 Freebase Type A and equal molar of H₂SO₄ in 0.5 mL EtOAc, followed by centrifugation and vacuum drying at 50 [symbol] .

XRPD and TGA/DSC results are displayed in FIG. 13, Table 7, and FIG. 14. TGA result showed a weight loss of 3.62% up to 150.0 [symbol] , and DSC result showed one endothermic peak at 85.6 [symbol] (peak) and one exothermic peak at 174.0 [symbol] (peak). HPLC/IC showed the molar ratio of acid to freebase was 1.2.

### Example 10: Preparation of Compound 1 Maleate Salt Type A

Compound 1 Maleate Type A was obtained via slurry 20 mg Compound 1 Freebase Type A and equal molar of maleic acid in 0.5 mL EtOAc, followed by centrifugation and vacuum drying at 50 [symbol] .

XRPD and TGA/DSC results are displayed in FIG. 15, Table 8, and FIG. 16. TGA result showed weight loss of 2.95% up to 150.0 [symbol] and 26.83% from 150 [symbol] to 225 [symbol] , and DSC result showed one endothermic peak at 177.6 [symbol] (peak) and one exothermic peak at 178.9 [symbol] (peak). ¹H NMR results (FIG. 17) showed the molar ratio of maleic acid to freebase was 1.0, and no solvent was observed. Combined with characterization results, Compound 1 Maleate Type A was speculated to be an anhydrate.

### Example 11: Preparation of Compound 1 Phosphate Salt Type A

Compound 1 Phosphate Type A was obtained via slurry 20 mg Compound 1 Freebase Type A and equal molar of H₃PO₄ in 0.5 mL acetone/H₂O (19:1, v/v), followed by centrifugation and vacuum drying at 50 [symbol] .

XRPD, TGA and DSC results are shown in FIG. 18, Table 9, and FIG. 19. TGA result showed a weight loss of 13.70% up to 150.0, and DSC result showed two endothermic peaks at 68.4 [symbol] , 80.9 [symbol] (peak) and one exothermic peak at 182.8 [symbol] (peak). HPLC/IC showed the molar ratio of acid to freebase was 1.1.

### Example 12: Preparation of Compound 1 Citrate Salt Type A

Compound 1 Citrate Type A was obtained via slurry 20 mg Compound 1 Freebase Type A and equal molar of citric acid in 0.5 mL EtOAc, followed by centrifugation and vacuum drying at 50 [symbol] .

XRPD, TGA, and DSC results are shown in FIG. 20, Table 10, and FIG. 21. TGA result showed weight loss of 0.73% up to 150.0 [symbol] and 36.11% from 150 [symbol] to 225 [symbol] , and DSC result showed two endothermic peaks at 143.0 [symbol] , 179.7 [symbol] . (peak) and one exothermic peak at 176.7 [symbol] (peak). ¹H NMR results (FIG. 22) showed the molar ratio of acid to freebase was 1.0:1, and EtOAc to freebase was 0.03:1 (~0.5 wt %).

### Example 13: Preparation of Compound 1 L-Malate Salt Type A

Compound 1 L-malate Type A was obtained via slurry 20 mg Compound 1 Freebase Type A and equal molar of L-malic acid in 0.5 mL EtOAc, followed by centrifugation and vacuum drying at 50 [symbol] .

XRPD, TGA, and DSC results are shown in FIG. 23, Table 11, and FIG. 24. TGA result showed weight loss of 1.85% up to 150.0 [symbol] and 33.44% from 150 [symbol] to 250 [symbol] , and DSC result showed one endothermic peak at 189.6 [symbol] (peak) and one exothermic peak at 159.5 [symbol] (peak). ¹H NMR results (FIG. 25) showed the molar ratio of L-malic acid to freebase was 1.3:1, and no EtOAc was observed.

### Example 14: Preparation of Compound 1 Succinate Salt Type A

Compound 1 Succinate Type A was obtained via slurry 20 mg Compound 1 Freebase Type A and equal molar of succinic acid in 0.5 mL EtOAc, followed by centrifugation and vacuum drying at 50

[symbol]

XRPD, TGA, and DSC results are shown in FIG. 26, Table 12, and FIG. 27. TGA result showed step weight loss of 3.28% up to 150.0 [symbol] and 27.31% from 150 [symbol] to 225 [symbol] , and DSC result showed one exothermic peak at 187.3 [symbol] (peak). ¹H NMR results (FIG. 28) showed the molar ratio of succinic acid to freebase was 0.8:1, and no solvent was observed.

### Example 15: Preparation of Compound 1 Tosylate Salt Type A

Compound 1 Tosylate salt Type A was obtained via slurry 20 mg Compound 1 Freebase Type A and equal molar of p-toluenesulfonic acid in 0.5 mL acetone/H₂O (19:1, v/v). The suspension was centrifuged and vacuum dried at 50° C.

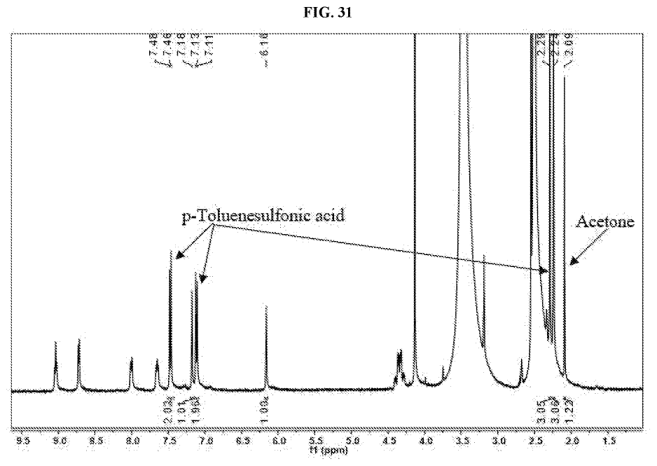

XRPD, TGA, and DSC results are shown in FIG. 29, Table 13, and FIG. 30. TGA result showed a weight loss of 6.08% up to 200.0 [symbol] , DSC result showed one endothermic peak at 88.3 [symbol] (peak) and one exothermic peak at 174.5 [symbol] (peak). ¹H NMR results (FIG. 31) showed the molar ratio of p-toluenesulfonic acid to freebase was 1.0:1, and the molar ratio of acetone to freebase was 0.2:1 (~2.0 wt %).

### Example 16: Preparation of Compound 1 Tosylate Salt Type B

Compound 1 Tosylate salt B was obtained via slurry 20 mg Compound 1 Freebase Type A and equal molar of p-toluenesulfonic acid in 0.5 mL EtOAc. The suspension was centrifuged and vacuum dried at 50 [symbol] .

XRPD, TGA, and DSC results are shown in FIG. 32, Table 14, and FIG. 33. TGA result showed a weight loss of 3.23% up to 150.0° C., DSC result showed two exothermic peaks at 166.1 [symbol] and 181.2 [symbol] (peak). ¹H NMR results (FIG. 34) showed molar ratio of p-toluenesulfonic acid to freebase was 1.2:1, and no EtOAc was observed.

### Example 17: Preparation of Compound 1 Tosylate Salt Type C

Compound 1 Tosylate salt Type C was obtained via slurry 20 mg Compound 1 Freebase Type A and equal molar of p-toluenesulfonic acid in 0.5 mL IPA. The suspension was centrifuged and vacuum dried at 50 [symbol] .

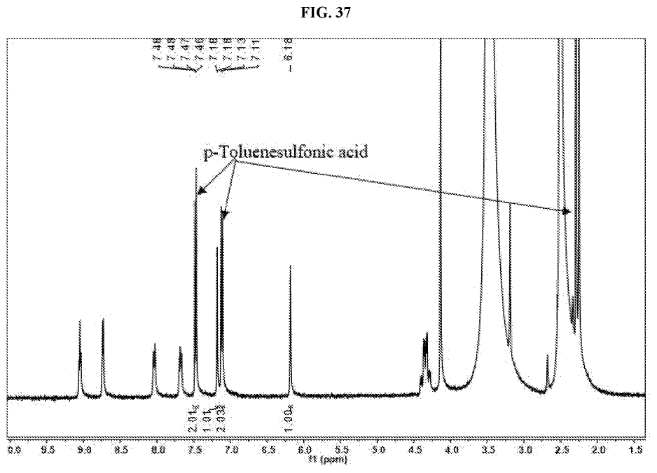

XRPD, TGA, and DSC results were displayed in FIG. 35, Table 15, and FIG. 36. TGA result showed a weight loss of 2.62% up to 150.0 [symbol] , DSC result showed two exothermic peaks at 174.5 [symbol] and 192.8 [symbol] (peak). ¹H NMR results (FIG. 37) showed the molar ratio of p-toluenesulfonic acid to freebase was 1.0:1, and no IPA was observed.

### Example 18: Preparation of Compound 1 Mesylate Salt Type A

Compound 1 Mesylate Type A was obtained via slurry 20 mg Compound 1 Freebase Type A and equal molar of methanesulfonic acid in 0.5 mL EtOAc, followed by centrifugation and vacuum drying at 50 [symbol] .

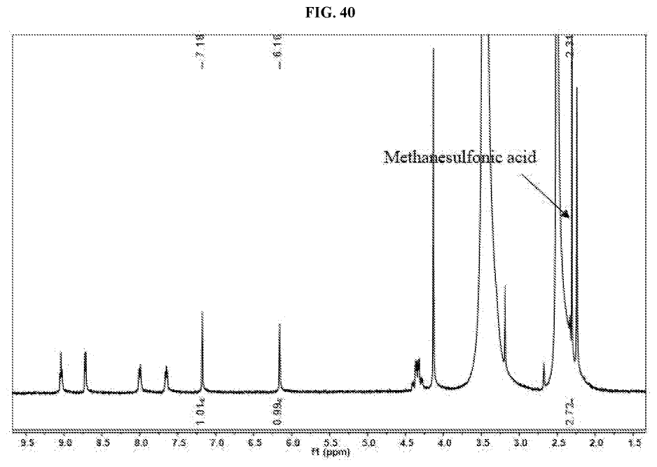

XRPD and TGA/DSC results are displayed in FIG. 38, Table 16, and FIG. 39. TGA result showed step weight loss of 8.77% up to 200.0 [symbol] and 21.12% from 200 [symbol] to 275 [symbol] , and DSC result showed two endothermic peaks at 91.7 [symbol] , 240.2 [symbol] (peak) and three exothermic peaks at 173.5 [symbol] , 179.7 [symbol] and 255.8 [symbol] (peak). ¹H NMR results (FIG. 40) showed the molar ratio of methanesulfonic acid to freebase was 0.9:1, and no EtOAc was observed.

### Example 19: Preparation of Compound 1 Besylate Salt Type A

Compound 1 Besylate Type A was obtained via slurry 20 mg Compound 1 Freebase Type A and equal molar of benzenesulfonic acid in 0.5 mL EtOAc, followed by centrifugation and vacuum drying at 50 [symbol] .

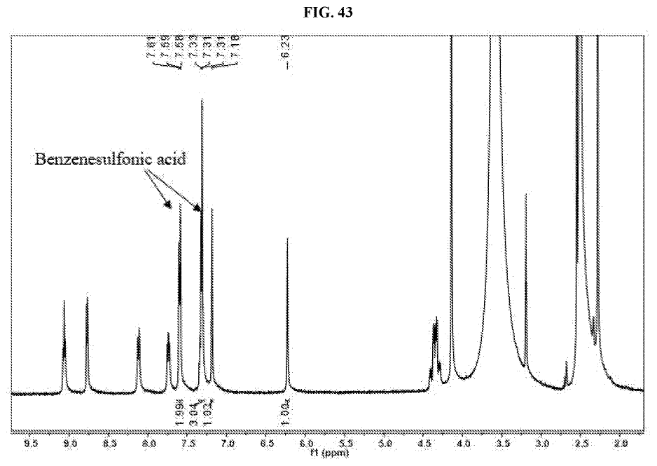

XRPD and TGA/DSC results are displayed in FIG. 41, Table 17, and FIG. 42. TGA result showed a weight loss of 4.70% up to 150.0 [symbol] , and DSC result showed two endothermic peaks at 39.7 [symbol] , 124.3 [symbol] (peak) and one exothermic peak at 168.7 [symbol] (peak). ¹H NMR results (FIG. 43) showed the molar ratio of benzenesulfonic acid to freebase was 1.0:1, and no EtOAc was observed.

### Example 20: Preparation of Compound 1 Oxalate Salt Type A

Compound 1 Oxalate Type A was obtained via slurry 20 mg Compound 1 Freebase Type A and equal molar of oxalic acid in 0.5 mL EtOAc, followed by centrifugation and vacuum drying at 50 [symbol] .

XRPD, TGA, and DSC results are shown in FIG. 44, Table 18, and FIG. 45. TGA result showed step weight loss of 5.97% up to 150.0 [symbol] , 10.58% from 150.0 [symbol] to 200 [symbol] and 13.11% from 200 [symbol] to 250 [symbol] , DSC result showed one endothermic peak at 251.0 [symbol] (peak) and one exothermic peak at 174.3 [symbol] (peak). ¹H NMR results showed no EtOAc was observed. HPLC/IC showed the molar ratio of acid to freebase was 0.9.

### Example 21: Preparation of Compound 1 Oxalate Salt Type B

Compound 1 Oxalate Type B was obtained via slurry 20 mg Compound 1 Freebase Type A and equal molar of freebase oxalic acid in IPA, followed by centrifugation and vacuum drying at 50° C.

XRPD, TGA and DSC results are shown in FIG. 46, Table 19, and FIG. 47. TGA result showed step weight loss of 4.89% up to 150.0 [symbol] , 5.69% from 150.0 [symbol] to 200 [symbol] and 11.34% from 200 [symbol] to 250 [symbol] , DSC result showed two endothermic peaks at 78.1 [symbol] , 247.3 [symbol] (peak) and one exothermic peak at 169.0 [symbol] (peak). ¹H NMR results showed no IPA was observed. HPLC/IC showed the molar ratio of acid to freebase was 0.6.

### Example 22: Preparation of Compound 1 Esylate Salt Type A

Compound 1 Esylate Type A was obtained via slurry 20 mg Compound 1 Freebase Type A and equal molar of ethanesulfonic acid in 0.5 mL EtOAc, followed by centrifugation and vacuum drying at 50 [symbol] .

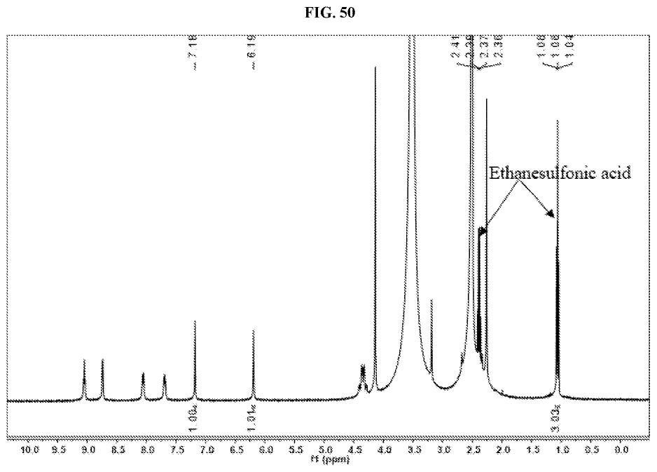

XRPD, TGA, and DSC results are shown in FIG. 48, Table 20, and FIG. 49. TGA result showed a weight loss of 3.41% up to 150.0 [symbol] , DSC result showed three endothermic peaks at 101.7 [symbol] , 151.7 [symbol] , 155.0 [symbol] (peak) and three exothermic peaks at 161.9 [symbol] , 176.0 [symbol] , 152.5 [symbol] (peak). ¹H NMR results (FIG. 50) showed the molar ratio of ethanesulfonic acid to freebase was 1.0:1, and no EtOAc was observed.

### Example 23: Preparation of Compound 1 Esylate Salt Type B

Compound 1 Esylate Type B was obtained via slurry 20 mg Compound 1 Freebase Type A and equal molar of ethanesulfonic acid in 0.5 mL IPA, followed by centrifugation and vacuum drying at 50 [symbol] .

XRPD, TGA, and DSC results are shown in FIG. 51, Table 21, and FIG. 52. TGA result showed a weight loss of 3.05% up to 150.0 [symbol] , DSC result showed two endothermic peaks at 80.9 [symbol] , 152.8 [symbol] (peak) and five exothermic peaks at 159.7 [symbol] , 178.9 [symbol] , 196.9 [symbol] , 206.0 [symbol] , 250.0 [symbol] (peak). ¹H NMR results (FIG. 53) showed the molar ratio of ethanesulfonic acid to freebase was 1.0:1, and no IPA was observed.

### Example 24: Hygroscopicity Evaluation

Compound 1 Freebase Type A/B/C, Compound 1 maleate salt Type A, and Compound 1 citrate salt Type A were evaluated for hygroscopicity. DVS isotherm plots were collected at 25 [symbol] between 0% RH and 95% RH.

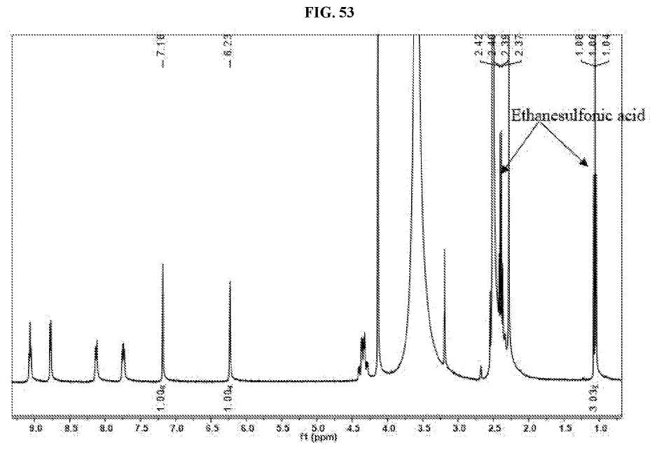

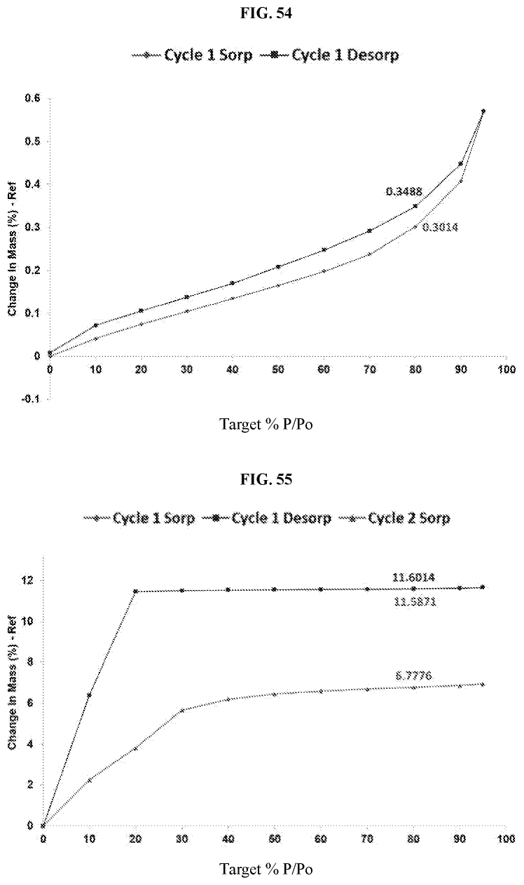

Compound 1 Freebase Type A: a water uptake of ~0.3% up to 80% RH was observed, indicating the sample was slightly hygroscopic (FIG. 54).

Compound 1 Freebase Type B: a water uptake of ~11.6% up to 80% RH was observed, and the water uptake decreased sharply as the humidity lower than 20% RH (FIG. 55).

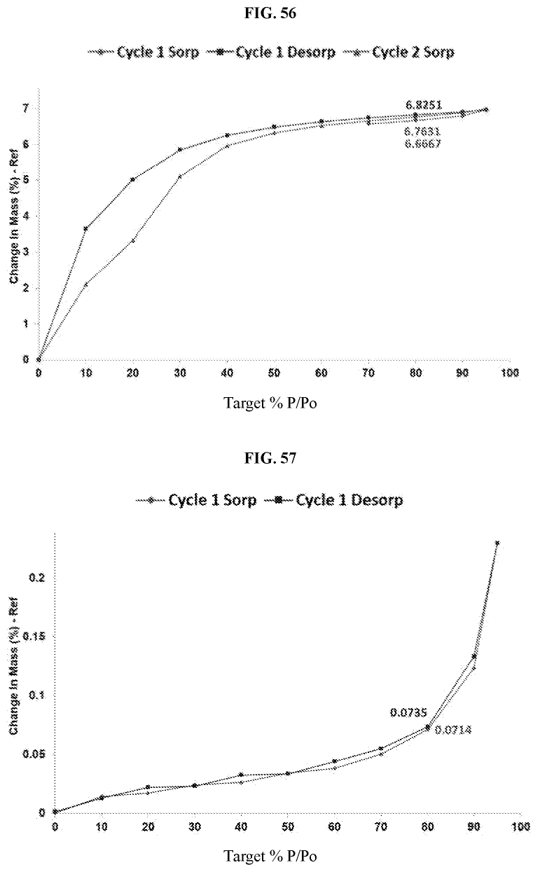

Compound 1 Freebase Type C: a water uptake of ~6.7% up to 80% RH was observed (FIG. 56).

Compound 1 maleate salt Type A: a water uptake of ~0.07% up to 80% RH was observed, indicating the sample was non-hygroscopic (FIG. 57).

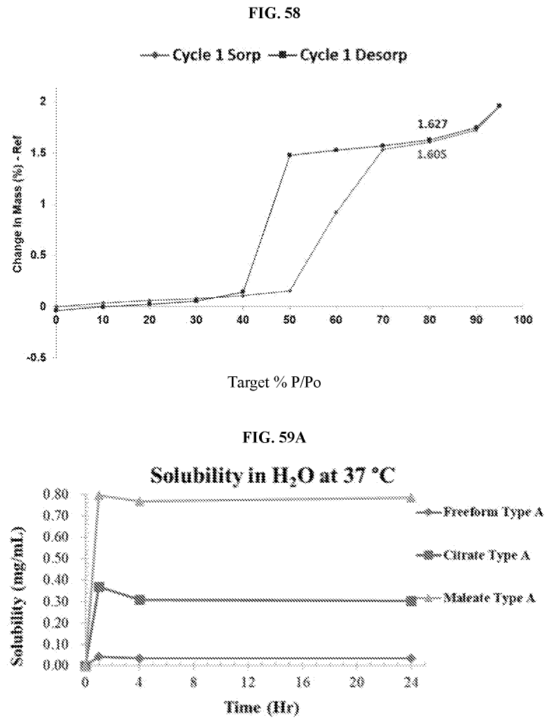

Compound 1 citrate salt Type A: a water uptake of 1.6% up to 80% RH indicating the sample was slightly hygroscopic and a platform was observed when humility was above 70% RH, which may indicate form change at high humidity (FIG. 58).

### Example 25: Kinetic Solubility Evaluation

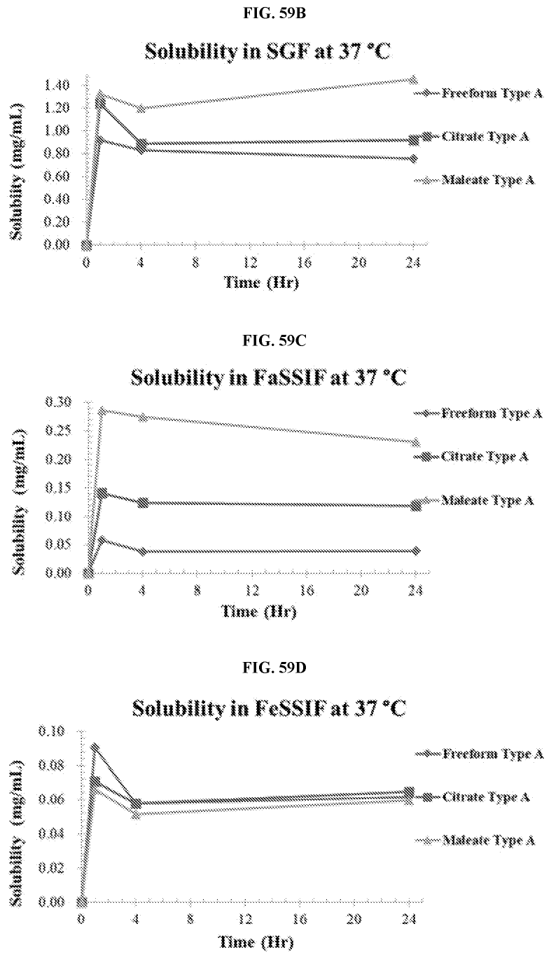

Kinetic solubility of Compound 1 Freebase Type A, Compound 1 maleate salt Type A, and Compound 1 citrate salt Type A were measured in water, SGF (Simulated Gastric Fluid), FaSSIF (Fasted State Simulated Intestinal Fluid) and FeSSIF (Fed State Simulated Intestinal Fluid) at 37 [symbol] with solid loading of ~10 mg/mL (calculated as freebase). The results are presented in FIG. 59A, FIG. 59B, FIG. 59C, and FIG. 59D. Compared with Compound 1 Freebase Type A, Compound 1 maleate salt Type A and Compound 1 citrate salt Type A showed improved solubility in H₂O, SGF and FaSSIF. Compound 1 maleate salt Type A showed higher solubility than Compound 1 citrate salt Type A in H₂O, SGF and FaSSIF.

### Example 26: Preparation of 1 mg Capsules

Compound 1 freebase Type A or Compound 1 maleate salt Type A was sieved with 40 mesh sieves, followed by mixing with the same amount of diluent, disintegrant and lubricant to prepare 1000 capsules. The strength of these capsules is 1 mg, and the capsules are packaged in aluminum blister foils. The stability of 1 mg capsules was investigated at 40° C./75% RH and 60° C., and the results were in Table 26.

TABLE 26

| | | | Total impurity (%) | | |
|---|---|---|---|---|---|
| No | Salt form | Condition | 0 d | 5 d | 10 d |
| 1 | Maleate Type A | 40° C./75% RH | 0.10 | / | 2.0 |
| 2 | Free form Type A | 40° C./75% RH | 0.21 | | 0.95 |
| 3 | Maleate Type A | 60° C. | 0.10 | 12.6 | / |
| 4 | Free form Type A | 60° C. | 0.21 | 4.4 | 8.04 |

It is understood that the examples and embodiments described herein are for illustrative purposes only and that various modifications or changes in light thereof will be suggested to persons skilled in the art and are to be included within the spirit and purview of this application and scope of the appended claims. A person of skill would understand that although the above diffractometer and/or diffractometer parameters were used in the examples, other types of diffractometers or parameters can also be used. Furthermore, other wavelengths can be used and converted to the Cu Kα.

What is claimed is:

1. A solid state form of 5-methoxy-1-(2-methylpyridin-3-yl)-4-(prop-2-yn-1-ylamino)-7-(trifluoromethyl) quinazolin-2 (1H)-one: 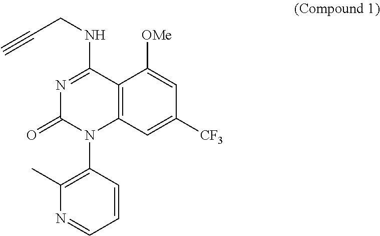 [SMILES: C#CCNc1nc(=O)n(-c2cccnc2C)c2cc(C(F)(F)F)cc(OC)c12]
or a pharmaceutically acceptable salt thereof.

2. The solid state form of claim 1, wherein the solid state form is a crystalline form.

3. The solid state form of claim 1 or 2, wherein the solid state form is crystalline Compound 1 freebase Type A.

4. The solid state form of claim 1 or 2, wherein the solid state form is crystalline Compound 1 freebase Type B, crystalline Compound 1 freebase Type C, crystalline Compound 1 HCl salt Type A, crystalline Compound 1 HCl salt Type B, crystalline Compound 1 HCl salt Type C, crystalline Compound 1 sulfate salt Type A, crystalline Compound 1 phosphate salt Type A, crystalline Compound 1 L-malate salt Type A, crystalline Compound 1 succinate salt Type A, crystalline Compound 1 tosylate salt Type A, crystalline Compound 1 tosylate salt Type B, crystalline Compound 1 tosylate salt Type C, crystalline Compound 1 mesylate salt Type A, crystalline Compound 1 besylate salt Type A, crystalline Compound 1 oxalate salt Type A, crystalline Compound 1 oxalate salt Type B, crystalline Compound 1 esylate salt Type A, or crystalline Compound 1 esylate salt Type B.

5. The solid state form of claim 1 or 2, wherein the solid state form is Compound 1 maleate salt.

6. The solid state form of claim 1 or 2, wherein the solid state form is crystalline Compound 1 maleate salt Type A.

7. The solid state form of claim 1 or 2, wherein the solid state form is crystalline Compound 1 citrate salt Type A.

8. The crystalline form of claim 2, wherein the crystalline form is Compound 1 freebase Type A characterized as having at least one of the following properties:
(a) an X-Ray powder diffraction (XRPD) pattern substantially the same as shown in FIG. 1 as measured using Cu Kα.radiation;
(b) an X-Ray powder diffraction (XRPD) pattern with peaks at 8.7±0. [symbol] 2θ and 14.0±0. [symbol] 2θ as measured using Cu Kα.radiation;
(c) a Differential Scanning calorimetry (DSC) thermogram substantially the same as shown in FIG. 2;
(d) a Thermogravimetric Thermal Analysis (TGA) thermogram substantially the same as shown in FIG. 2; or
(e) combinations thereof.

9. The crystalline form of claim 8, wherein the crystalline form has an X-ray powder diffraction (XRPD) pattern with characteristic peaks found in Table 1 as measured using Cu Kα.radiation.

10. The crystalline form of claim 8, wherein the crystalline form has an X-ray powder diffraction (XRPD) pattern with peaks at 8.7±0. [symbol] 2θ and 14.0±0.2 [symbol] 2θ as measured using Cu Kα.radiation.

11. The crystalline form of claim 9 or 10, wherein the X-ray powder diffraction (XRPD) pattern further comprises peaks at 7.7±0. [symbol] 2θ, 9.5±0. [symbol] 2θ, and 11.6±0. [symbol] 2θ as measured using Cu Kα.radiation.

12. The crystalline form of any one of claims 8, 10-11, wherein the X-ray powder diffraction (XRPD) pattern further comprises peaks at 17.8±0. [symbol] 2θ, 20.8±0. [symbol] 2θ, and 24.2±0. [symbol] 2θ as measured using Cu Kα.radiation.

13. The crystalline form of claim 2, wherein the crystalline form is Compound 1 Maleate salt Type A characterized as having at least one of the following properties:
(a) an X-Ray powder diffraction (XRPD) pattern substantially the same as shown in FIG. 15 as measured using Cu Kα.radiation;
(b) an X-Ray powder diffraction (XRPD) pattern with peaks at 9.6±0. [symbol] 2θ, 15.6±0. [symbol] 2θ, and 16.6±0. [symbol] 2θ as measured using Cu Kα.radiation;
(c) a Differential Scanning calorimetry (DSC) thermogram substantially the same as shown in FIG. 16;
(d) a Thermogravimetric Thermal Analysis (TGA) thermogram substantially the same as shown in FIG. 16; or
(e) combinations thereof.

14. The crystalline form of claim 13, wherein the crystalline form has an X-ray powder diffraction (XRPD) pattern with peaks found in Table 8 as measured using Cu Kα.radiation.

15. The crystalline form of claim 13, wherein the crystalline form has an X-ray powder diffraction (XRPD) pattern with peaks at 9.6±0. [symbol] 2θ, 15.6±0. [symbol] 2θ, and 16.6±0. [symbol] 2θ as measured using Cu Kα.radiation.

16. The crystalline form of claim 13 or 15, wherein the X-ray powder diffraction (XRPD) pattern further comprises peaks at 13.0±0. [symbol] 2θ, 19.7±0. [symbol] 2θ, and 20.6±0. [symbol] 2θ as measured using Cu Kα.radiation.

17. The crystalline form of any one of claim 13, 15, or 16, wherein the X-ray powder diffraction (XRPD) pattern further comprises peaks at 7.1±0. [symbol] 2θ, 23.0±0. [symbol] 2θ, 23.7±0. [symbol] 2θ, and 24.5±0. [symbol] 2θ as measured using Cu Kα.radiation.

18. The crystalline form of any one of claims 12-17, wherein the crystalline form is anhydrous.

19. The crystalline form of claim 2, wherein the crystalline form is Compound 1 Citrate salt Type A characterized as having at least one of the following properties:
(a) an X-Ray powder diffraction (XRPD) pattern substantially the same as shown in FIG. 20 as measured using Cu Kα.radiation;
(b) an X-Ray powder diffraction (XRPD) pattern with peaks at 7.6±0. [symbol] 2θ, 13.1±0. [symbol] 2θ, and 14.6±0. [symbol] 2θ as measured using Cu Kα.radiation;
(c) a Differential Scanning calorimetry (DSC) thermogram substantially the same as shown in FIG. 21;
(d) a Thermogravimetric Thermal Analysis (TGA) thermogram substantially the same as shown in FIG. 21; or
(e) combinations thereof.

20. The crystalline form of claim 19, wherein the crystalline form has an X-ray powder diffraction (XRPD) pattern with peaks found in Table 10 as measured using Cu Kα.radiation.

21. The crystalline form of claim 19, wherein the crystalline form has an X-ray powder diffraction (XRPD) pattern with peaks at 7.6±0. [symbol] 2θ, 13.1±0. [symbol] 2θ, and 14.6±0. [symbol] 2θ as measured using Cu Kα.radiation.

22. The crystalline form of claim 19 or 21, wherein the X-ray powder diffraction (XRPD) pattern further comprises peaks at 17.2±0. [symbol] 2θ, 18.3±0. [symbol] 2θ, and 20.1±0. [symbol] 2θ as measured using Cu Kα.radiation.

23. A pharmaceutical composition comprising a crystalline form of any one of claims 2-22 and at least one pharmaceutically acceptable excipient.

24. A method of treating cancer in a mammal in need thereof, comprising administering to the mammal a crystalline form of any one of claims 2-22 or pharmaceutical composition of claim 23.

25. The method of claim 24, wherein the cancer is a MTAP-deficient cancer.

26. The method of claim 24 or claim 25, wherein the cancer is selected from liver cancer, colon cancer, pancreatic cancer, prostate cancer, lung cancer, breast cancer, gastrointestinal stromal tumor, biliary tract cancer, acute lymphoblastic leukemia (ALL) B-lineage, lymphoma, or T cell leukemia.
